SEQUENCE LISTING (63 sequences)

SEQ ID NO: 1 - protein, 14 aa, Homo sapiens
  VARIANT 2: note = X in position 2 is S or K
  VARIANT 3: note = X in position 3 is D, S or A
  VARIANT 5: note = X in position 5 is Y, S or A
  VARIANT 7: note = X in position 7 is S or N
  VARIANT 9: note = X in position 9 is V or I
FXXYXMXWXRQAPG

SEQ ID NO: 2 - protein, 14 aa, Homo sapiens
  SITE 3: note = X in position 3 is D or S
  VARIANT 5: note = X in position 5 is Y, S or A
  VARIANT 7: note = X in position 7 is S or N
FSXYXMXWVRQAPG

SEQ ID NO: 3 - protein, 14 aa, Homo sapiens
  VARIANT 3: note = X in position 3 is D or S
  VARIANT 5: note = X in position 5 is Y or S
  VARIANT 7: note = X in position 7 is S or N
FSXYXMXWVRQAPG

SEQ ID NO: 4 - protein, 19 aa, Homo sapiens
  VARIANT 2: note = X in position 2 is G or A
  VARIANT 5: note = X in position 5 is W, G or N
  VARIANT 6: note = X in position 6 is S or T
  VARIANT 7: note = X in position 7 is S or G
  VARIANT 8: note = X in position 8 is R or G
  VARIANT 10: note = X in position 10 is K, T or I
  VARIANT 11: note = X in position 11 is G, Y, H or D
  VARIANT 12: note = X in position 12 is Y or F
  VARIANT 9: note = X in position 9 is D, S or Y
SXISXXXXXXXXADSVKGR

SEQ ID NO: 5 - protein, 19 aa, Homo sapiens
  VARIANT 2: note = X in position 2 is G or A
  VARIANT 5: note = X in position 5 is W, G or N
  VARIANT 6: note = X in position 6 is S or T
  VARIANT 7: note = X in position 7 is S or G
  VARIANT 8: note = X in position 8 is R or G
  VARIANT 10: note = X in position 10 is K, T or I
  VARIANT 9: note = X in position 9 is D, S or Y
  VARIANT 11: note = X in position 11 is G, Y, H or D
SXISXXXXXXXYADSVKGR

SEQ ID NO: 6 - protein, 19 aa, Homo sapiens
  VARIANT 2: note = X in position 2 is G or A
  VARIANT 5: note = X in position 5 is W or G
  VARIANT 7: note = X in position 7 is S or G
  VARIANT 8: note = X in position 8 is R or G
  VARIANT 10: note = X in position 10 is K or T
  VARIANT 11: note = X in position 11 is G or Y, H or D
  VARIANT 9: note = X in position 9 is D or S
SXISXSXXXXXYADSVKGR

SEQ ID NO: 7-8 - skipped (2 entries)

SEQ ID NO: 9 - protein, 15 aa, Homo sapiens
  VARIANT 2: note = X in position 2 is T or S
  VARIANT 10: note = X in position 10 is A or S
  VARIANT 11: note = X in position 11 is G or N
  VARIANT 13: note = X in position 13 is D or V
  VARIANT 14: note = X in position 14 is V or Y
  VARIANT 15: note = X in position 15 i s H or none
CXGSSSNIGXXYXXX

SEQ ID NO: 10 - protein, 15 aa, Homo sapiens
CTGSSSNIGAGYDVH

SEQ ID NO: 11 - protein, 7 aa, Homo sapiens
  VARIANT 1: note = X in position 1 is G, R, or D
  VARIANT 3: note = X in position 3 is D, N, or S
  VARIANT 4: note = X in position 4 is N, Q or K
XNXXRPS

SEQ ID NO: 12 - protein, 7 aa, Homo sapiens
  VARIANT 1: note = X in position 1 is G or R
  VARIANT 3: note = X in position 3 is D or N
  VARIANT 4: note = X in position 4 is N or Q
XNXXRPS

SEQ ID NO: 13 - protein, 13 aa, Homo sapiens
  VARIANT 2: note = X in position 2 is A or Q
  VARIANT 3: note = X in position 3 is V, A or S
  VARIANT 4: note = X in position 4 is W or Y
  VARIANT 9: note = X in position 9 is N or S
  VARIANT 11: note = X in position 11 is V, W or P
  VARIANT 13: note = X in position 13 is V or none
  VARIANT 6: note = X in position 6 is D or S
CXXXDXSLXGXVX

SEQ ID NO: 14 - protein, 12 aa, Homo sapiens
  VARIANT 3: note = X in position 3 is V or A
  VARIANT 11: note = X in position 11 is V or W
CAXWDDSLNGXV

SEQ ID NO: 15 - protein, 14 aa, Homo sapiens
FSDYYMSWVRQAPG

SEQ ID NO: 16 - protein, 19 aa, Homo sapiens
SGISWSSRDKGYADSVKGR

SEQ ID NO: 17 - protein, 7 aa, Homo sapiens
TTDLARY

SEQ ID NO: 18 - protein, 7 aa, Homo sapiens
GNDNRPS

SEQ ID NO: 19 - protein, 12 aa, Homo sapiens
CAVWDDSLNGVV

SEQ ID NO: 20 - protein, 114 aa, Homo sapiens
EVQLLESGGGLVQPGGSLRLSCAASGFTFSDYYMSWVRQAPGKGLEWVSGISWSSRDKGYADSVKGRFTISRDNSKNTLYLQMNSLRAEDTAVYYCTTDLARYWGQGTLVTVSS

SEQ ID NO: 21 - protein, 112 aa, Homo sapiens
QSVLTQPPSASGTPGQRVTISCTGSSSNIGAGYDVHWYQQLPGTAPKLLIYGNDNRPSGVPDRFSGSKSGTSASLAISGLRSEDEADYYCAVWDDSLNGVVFGGGTKLTVLG

SEQ ID NO: 22 - protein, 14 aa, Homo sapiens
FSSYSMNWVRQAPG

SEQ ID NO: 23 - protein, 19 aa, Homo sapiens
SAISGSGGSTYYADSVKGR

SEQ ID NO: 24 - protein, 13 aa, Homo sapiens
ARDRVEMNQWLAD

SEQ ID NO: 25 - protein, 7 aa, Homo sapiens
RNNQRPS

SEQ ID NO: 26 - protein, 12 aa, Homo sapiens
CAAWDDSLNGWV

SEQ ID NO: 27 - protein, 120 aa, Homo sapiens
EVQLLESGGGLVQPGGSLRLSCAASGFTFSSYSMNWVRQAPGKGLEWVSAISGSGGSTYYADSVKGRFTISRDNSKNTLYLQMNSLRAEDTAVYYCARDRVEMNQWLADWGQGTLVTVSS

SEQ ID NO: 28 - protein, 112 aa, Homo sapiens
QSVLTQPPSASGTPGQRVTISCTGSSSNIGAGYDVHWYQQLPGTAPKLLIYRNNQRPSGVPDRFSGSKSGTSASLAISGLRSEDEADYYCAAWDDSLNGWVFGGGTKLTVLG

SEQ ID NO: 29 - protein, 14 aa, Homo sapiens
FSSYAMSWVRQAPG

SEQ ID NO: 30 - protein, 19 aa, Homo sapiens
SGISGSGGYIHYADSVKGR

SEQ ID NO: 31 - protein, 13 aa, Homo sapiens
ATYSSGLHDAFDI

SEQ ID NO: 32 - protein, 7 aa, Homo sapiens
DNNKRPS

SEQ ID NO: 33 - protein, 120 aa, Homo sapiens
EVQLLESGGGLVQPGGSLRLSCAASGFTFSSYAMSWVRQAPGKGLEWVSGISGSGGYIHYADSVKGRFTISRDNSKNTLYLQMNSLRAEDTAVYYCATYSSGLHDAFDIWGQGTLVTVSS

SEQ ID NO: 34 - protein, 112 aa, Homo sapiens
QSVLTQPPSASGTPGQRVTISCTGSSSNIGAGYDVHWYQQLPGTAPKLLIYDNNKRPSGVPDRFSGSKSGTSASLAISGLRSEDEADYYCAAWDDSLNGWVFGGGTKLTVLG

SEQ ID NO: 35 - protein, 14 aa, Homo sapiens
FKAYSMSWIRQAPG

SEQ ID NO: 36 - protein, 19 aa, Homo sapiens
SGISNTGGSTDFADSVKGR

SEQ ID NO: 37 - protein, 15 aa, Homo sapiens
ARLGYSGYDDRGMDV

SEQ ID NO: 38 - protein, 14 aa, Homo sapiens
CSGSSSNIGSNYVY

SEQ ID NO: 39 - protein, 7 aa, Homo sapiens
GNSNRPS

SEQ ID NO: 40 - protein, 13 aa, Homo sapiens [sequence omitted]

SEQ ID NO: 41 - protein, 122 aa, Homo sapiens
EVQLLESGGGLVQPGGSLRLSCAASGFTFKAYSMSWIRQAPGKGLEWVSGISNTGGSTDFADSVKGRFTISRDNSKNTLYLQMNSLRAEDTAMYYCARLGYSGYDDRGMDVWGQGTLVTVSS

SEQ ID NO: 42 - protein, 112 aa, Homo sapiens
QSVLTQPPSASGTPGQRVTISCSGSSSNIGSNYVYWYQQLPGTAPKLLIYGNSNRPSGVPDRFSGSKSGTSASLAISGLRSEDEADYYCQSYDSSLSGPVVFGGGTKLTVLG

SEQ ID NO: 43 - protein, 330 aa, Homo sapiens [sequence omitted]

SEQ ID NO: 44 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 45 - DNA, 1335 bp, Homo sapiens
  source 1..1335: mol_type = unassigned DNA
gaggtgcagctgttggagtctgggggaggcttggtacagcctggggggtccctgagactctcctgtgcagcctctggattcaccttcagtgactactacatgagctgggtccgccaggctcccgggaaggggctggagtgggtctcaggcattagttggagtagtcgtgacaaaggctatgcggactctgtgaagggccgtttcaccatctccagagacaattccaagaacacgctgtatctgcaaatgaacagcctgagagccgaggacactgccgtgtattactgtaccacagatctcgctaggtactggggccagggtacactggtcaccgtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 46 - DNA, 654 bp, Homo sapiens
  source 1..654: mol_type = unassigned DNA
cagtctgtgctgactcagccaccctcagcgtctgggacccccgggcagagggtcaccatctcctgcactgggagcagctccaacatcggggcaggttatgatgtacactggtatcagcagctcccaggaacggcccccaaactcctcatctatggtaatgataaccggccctcaggggtccctgaccgattctctggctccaagtctggcacctcagcctccctggccatcagtgggctccggtccgaggatgaggctgattattactgtgcagtatgggatgacagcctgaatggtgtggtattcggcggaggaaccaagctgacggtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 47 - DNA, 1353 bp, Homo sapiens
  source 1..1353: mol_type = unassigned DNA
gaggtgcagctgttggagtctgggggaggcttggtacagcctggggggtccctgagactctcctgtgcagcctctggattcaccttcagtagctatagcatgaactgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacactgccgtgtattactgtgcgagagatcgggtagagatgaaccagtggctggccgactggggccagggtacactggtcaccgtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 48 - DNA, 654 bp, Homo sapiens
  source 1..654: mol_type = unassigned DNA
cagtctgtgctgactcagccaccctcagcgtctgggacccccgggcagagggtcaccatctcctgcactgggagcagctccaacatcggggcaggttatgatgtacactggtatcagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgaatggttgggtgttcggcggaggaaccaagctgacggtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 49 - DNA, 1353 bp, Homo sapiens
  source 1..1353: mol_type = unassigned DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattagtggcagcggtgggtacatacactatgcagactccgtgaagggccggttcaccatctccagagacaattccaagaacacgctgtatctgcaaatgaacagcctgagagccgaggacactgccgtgtattactgtgcgacctatagcagtggcctgcatgatgcttttgatatctggggccagggtacactggtcaccgtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 50 - DNA, 654 bp, Homo sapiens
  source 1..654: mol_type = unassigned DNA
cagtctgtgctgactcagccaccctcagcgtctgggacccccgggcagagggtcaccatctcctgcactgggagcagctccaacatcggggcaggttatgatgtacactggtatcagcagctcccaggaacggcccccaaactcctcatctatgacaataataagcgaccctcaggggtccctgaccgattctctggctccaagtctggcacctcagcctccctggccatcagtgggctccggtccgaggatgaggctgattattactgtgcagcatgggatgacagcctgaatggttgggtgttcggcggaggaaccaagctgacggtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 51 - DNA, 1359 bp, Homo sapiens
  source 1..1359: mol_type = unassigned DNA
gaggtgcagctgttggagtctgggggaggcttggtacagcctggggggtccctgagactctcctgtgcagcctctggattcaccttcaaagcctatagcatgagctggatccgccaggctccagggaaggggctggagtgggtctcaggtatcagtaacacgggaggtagcacagacttcgcagactccgtgaagggccggttcaccatctccagagacaattccaagaacacgctgtatctgcaaatgaacagcctgagagccgaggacactgccatgtattactgtgcgagattgggatatagtggctacgacgaccgtggtatggacgtctggggccaaggtacactggtcaccgtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 52 - DNA, 654 bp, Homo sapiens
  source 1..654: mol_type = unassigned DNA
cagtctgtgctgactcagccaccctcagcgtctgggacccccgggcagagggtcaccatctcttgttctggaagcagctccaacatcggaagtaattatgtgtactggtatcagcagctcccaggaacggcccccaaactcctcatctatggtaacagcaatcggccctcaggggtccctgaccgattctctggctccaagtctggcacctcagcctccctggccatcagtgggctccggtccgaggatgaggctgattattactgccagtcctatgacagcagcctgagtggtcctgtggtattcggcggaggaaccaagctgacggtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 53 - protein, 236 aa, Homo sapiens
MGWSCIILFLVATATGVHSQSVLTQPPSASGTPGQRVTISCTGSSSNIGAGYDVHWYQQLPGTAPKLLIYGNDNRPSGVPDRFSGSKSGTSASLAISGLRSEDEADYYCAVWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 54 - protein, 463 aa, Homo sapiens
MGWSCIILFLVATATGVHSEVQLLESGGGLVQPGGSLRLSCAASGFTFSDYYMSWVRQAPGKGLEWVSGISWSSRDKGYADSVKGRFTISRDNSKNTLYLQMNSLRAEDTAVYYCTTDLARYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 127 aa, Homo sapiens
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQTITFESFKENLKDFLLVIPFDCWEPVQE

SEQ ID NO: 56 - protein, 144 aa, Homo sapiens [sequence omitted]

SEQ ID NO: 57 - protein, 124 aa, Mus musculus
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 58 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 59 - DNA, 124 bp, synthetic construct
  source 1..124: mol_type = other DNA
ccacccactttttatagtaagtttttcacccataaataataaatacaataattaatttctcgtaaaagtagaaaatatattctaatttattgcacggtaaggaagtagaatcataaagaacagt

SEQ ID NO: 60 - DNA, 114 bp, synthetic construct
  source 1..114: mol_type = other DNA
tttattctatacttaaaaaatgaaaataaatacaaaggttcttgagggttgtgttaaattgaaagcgagaaataatcataaattatttcattatcgcgatatccgttaagtttg

SEQ ID NO: 61 - DNA, 40 bp, synthetic construct
  source 1..40: mol_type = other DNA
aaaaattgaaattttattttttttttttggaatataaata

SEQ ID NO: 62 - protein, 236 aa, Mus musculus
MGWSCIILFLVATATGVHSQSVLTQPPSASGTPGQRVTISCTGSSSNIGAGYDVHWYQQLPGTAPKLLIYRNNQRPSGVPDRFSGSKSGTSASLAISGLRSEDEADYYCAAWDDSLNGWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 63 - protein, 469 aa, Mus musculus
MGWSCIILFLVATATGVHSEVQLLESGGGLVQPGGSLRLSCAASGFTFSSYSMNWVRQAPGKGLEWVSAISGSGGSTYYADSVKGRFTISRDNSKNTLYLQMNSLRAEDTAVYYCARDRVEMNQWLADWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

(19) United States
(12) Patent Application Publication
(10) Pub. No.: US 2024/0374719 A1
(43) Pub. Date: Nov. 14, 2024

(54) NOVEL COMBINATIONS OF ANTIBODIES AND USES THEREOF

(71) Applicants: BIOINVENT INTERNATIONAL AB, Lund (SE); TRANSGENE SA, Illkirch-Graffenstaden (FR)

(72) Inventors: Björn FRENDÉUS, Lund (SE); Matilda REHN, Malmö (SE); Monika SEMMRICH, Malmö (SE); Linda MÅRTENSSON, Bjärred (SE); Jean-Baptiste MARCHAND, Obernai (FR)

(73) Assignees: BIOINVENT INTERNATIONAL AB, Lund (SE); TRANSGENE SA, Illkirch Graffenstaden (FR)

(21) Appl. No.: 18/691,186

(22) PCT Filed: Sep. 21, 2022
(86) PCT No.: PCT/EP2022/076268

§ 371 Date: Mar. 12, 2024

(30) Foreign Application Priority Data
Sep. 22, 2021 (EP) 21306310.0
Jan. 18, 2022 (EP) 22305050.1

(51) Int. Cl. A61K 39/395; A61K 35/768; A61K 39/00; A61P 35/00

(52) U.S. Cl. CPC A61K 39/3955; A61K 35/768; A61P 35/00; A61K 2039/505

(57) ABSTRACT

The present invention generally relates to a combination of an oncolytic virus capable of expressing a first antibody molecule that specifically binds to CTLA-4; and a second antibody molecule that specifically binds to PD-1 and/or PD-L1 and uses thereof in the treatment of cancer.

The present invention relates to a combination comprising: (i) an oncolytic virus capable of expressing a first antibody molecule that specifically binds to CTLA-4; and (ii) a second antibody molecule that specifically binds to PD-1 and/or PD-L1, wherein the combination is for use in treating a cancer comprising or consisting of a cold tumor in a patient. The invention also relates to the use of said combination in the manufacture of a medicament for treating cancer comprising or consisting of a cold tumor in a patient, and a method for treating a cancer comprising or consisting of a cold tumor in a patient comprising administering said combination.

Treatment with immune checkpoint blocking antibodies has transformed survival of patients with advanced solid cancers including metastatic melanoma, non-small cell lung cancer and mismatch-repair deficient cancers (Hodi et al., 2010; Larkin et al., 2015; Topalian et al., 2012).

However, a great unmet need remains since many patients fail to respond or acquire resistance to immune checkpoint blockade (ICB) (Sharma et al., 2017). Reasons for lack of efficacy are believed to include lack of, or inadequate, tumor infiltrating immune cells (TIL), most notably CD8+ T cells (Chen and Mellman, 2013; Gajewski et al., 2013). Paucity of chemotactic and inflammatory signals in the solid cancer tumor microenvironment (TME) is similarly thought to underlie resistance to CAR-T cell therapy (Wagner et al., 2020).

Identification of therapeutics that induce recruitment of inflammatory immune cells into “immune desert” or “immune excluded” tumors, translating into robust systemic adaptive antitumor immunity and CD8+ T cell infiltration with regression of primary and metastasized tumors, is therefore highly desired.

Intratumoral (i.t.) oncolytic virotherapy induces T cell infiltration and improves anti-PD-1 immunotherapy (Ribas et al., 2017). Combination therapy with anti-CTLA-4 and anti-PD-1 antibodies enhances efficacy compared to single agent ICB, likely through complementary mechanisms of systemic CD4+ and CD8+ T cell differentiation and tumor-localized modulation of T effector and regulatory T cells (Arce Vargas et al., 2018; Wei et al., 2019). However, tolerability issues with systemically administered anti-CTLA-4, including with the approved ipilimumab, have restricted clinical use (Postow et al., 2015). Efficacy and tolerability of systemic anti-CTLA-4 antibody therapy appear to be linked. Increasing ipilimumab dose enhanced both efficacy and side effects (Bertrand et al., 2015). Consistent with the central immune checkpoint function of CTLA-4, side effects may be severe and of systemic auto-immune nature (Tivol et al., 1995).

Interestingly, depletion of intra-tumoral (“i.t.”) Treg cells, which overexpress CTLA-4, relative to CD8⁺ and CD4⁺ effector T cells was recently reported to contribute to ipilimumab therapeutic activity, and Treg depletion-enhanced anti-CTLA-4 antibody variants showed improved therapeutic activity in tumor-bearing FcγR-humanized mice (Arce Vargas et al., 2018). These findings indicate that tumor-localized therapy with Treg depleting anti-CTLA-4 antibodies may provide powerful therapeutic activity with reduced side-effects compared to currently available anti-CTLA-4 therapies (Marabelle et al., 2013a; Marabelle et al., 2013b)—in particular when combined with validated and safe immunomodulators e.g. blockers of the PD-1/PD-L1 axis or oncolytic viruses.

Against that background, herein the inventors describe and characterize a Vaccinia virus (VV)-based oncolytic vector incorporating a full-length human recombinant anti-CTLA-4 antibody. The inventors also describe an oncolytic virus encoding a recently discovered full-length human recombinant anti-CTLA-4 antibody. This virally encoded novel human IgG1 CTLA-4 antibody (named “4-E03”) was identified using function-first screening for monoclonal antibodies (“mAbs”) and targets associated with superior Treg depleting activity. In a humanized mouse model characterized by human intratumoral-relevant CTLA-4 expression, 4-E03 IgG1 demonstrated enhanced Treg depletion compared to clinically validated ipilimumab. In contrast, 4-E03 shows similar potency in blocking CTLA-4:B7 interactions, and in overcoming CTLA-4-mediated suppression of effector T cell proliferation, compared with ipilimumab. In the present invention, a tumor-selective oncolytic Vaccinia vector was engineered to encode this novel, strongly Treg-depleting, checkpoint-blocking, anti-CTLA-4 antibody 4-E03 and GM-CSF (VV_{GM}-ahCTLA4, BT-001). Viruses encoding a matching Treg-depleting mouse surrogate antibody were additionally generated, enabling proof-of-concept studies in syngeneic immune competent mouse tumor models representing inflamed or immune excluded tumor microenvironments, sensitive or resistant to ICB.

This led to the unexpected finding that the oncolytic virus expressing an anti-CTLA-4 antibody synergised with anti-PD-1/PD-L1 antibodies to reject “cold” tumors” (also referred to herein as “cold immune tumors”). This was surprising since cold tumors are known to be resistant to systemic, intravenous, single agent or combined ICB with currently available anti-CTLA-4 and/or anti-PD-1, as were animals in the herein disclosed “cold tumor” mouse model.

As discussed in the Examples and herein, “cold” tumours are poorly T cell infiltrated. Surprisingly, the inventors have found that the combined treatment with the oncolytic virus expressing an anti-CTLA-4 antibody and an anti-PD-1/PD-L1 antibody induced a strong influx of T cells into “cold” tumors, which became similarly densely T cell rich as “hot” tumors.

The inventors' surprising findings provide further, beneficial therapeutic approaches for treating cancers which comprise or consist of “cold” tumors. As described herein, the invention relates generally to a combination comprising: (i) an oncolytic virus capable of expressing a first antibody molecule that specifically binds to CTLA-4; and (ii) a second antibody molecules that specifically binds to PD-1 and/or PD-L1, wherein the combination is for use in treating a cancer comprising or consisting of a cold tumor in a patient.

In a first aspect, the invention provides a combination comprising:
an oncolytic virus capable of expressing a first antibody molecule that specifically binds to CTLA-4; and
a second antibody molecule that specifically binds to PD-1 and/or PD-L1;
for use in treating cancer in a patient, wherein the cancer comprises or consists of a cold tumor.

In a second aspect, the invention provides the use of:
an oncolytic virus capable of expressing a nucleotide sequence encoding a first antibody molecule that specifically binds to CTLA-4; and
a second antibody molecule that specifically binds to PD-1 and/or PD-L1;
in the manufacture of a medicament for treating cancer in a patient, wherein the cancer comprises or consists of a cold tumor.

In a third aspect, the invention provides a method for treating cancer in a patient, wherein the cancer comprises or consists of a cold tumor, the method comprising administering to the patient:
an oncolytic virus capable of expressing a first antibody molecule that specifically binds to CTLA-4; and
a second antibody molecule that specifically binds to PD-1 and/or PD-L1.

In a fourth aspect, the invention provides an oncolytic virus capable of expressing a first antibody molecule that specifically binds to CTLA-4, for use in combination with a second antibody molecule that specifically binds to PD-1 and/or PD-L1; for treating cancer in a patient, wherein the cancer comprises or consists of a cold tumor.

As discussed above and demonstrated in the accompanying Examples, in the above aspects of the invention, treatment with an oncolytic virus capable of expressing a first antibody molecule that specifically binds to CTLA-4, and a second antibody that specifically binds to PD-1 and/or PD-L1 causes an influx of T cells into the cold tumor of the cancer in the patient. That results in an increase in the number and/or density of T cells in the cold tumour of the cancer of the patient, such that the tumour becomes similarly densely T cell rich as hot tumors. In an embodiment of the invention, the number and/or density of T cells in the cold tumour of the cancer of the patient increases by approximately 5-fold to 25-fold or more, for example by approximately 5-fold, or 6-fold, or 7-fold, or 8-fold, or 9-fold, or 10-fold, or 11-fold, or 12-fold, or 13-fold, or 14-fold, or 15-fold, or 16-fold, or 17-fold, or 18-fold, or 19-fold, or 20-fold, or 21-fold, or 22-fold, or 23-fold, or 24-fold, or 25-fold, or more.

Thus, in a further aspect, the invention provides a combination comprising:
an oncolytic virus capable of expressing a first antibody molecule that specifically binds to CTLA-4; and
a second antibody molecule that specifically binds to PD-1 and/or PD-L1;
for use increasing the number and/or density of T cells in a cold tumour in a cancer in a patient, and/or mediating an influx of T cells into a cold tumour in a cancer in a patient. It will be appreciated that, by doing so, the invention treats the cancer in the patient. In an embodiment of the invention, the number and/or density of T cells in the cold tumour of the cancer of the patient increases by approximately 5-fold to 25-fold or more, for example by approximately 5-fold, or 6-fold, or 7-fold, or 8-fold, or 9-fold, or 10-fold, or 11-fold, or 12-fold, or 13-fold, or 14-fold, or 15-fold, or 16-fold, or 17-fold, or 18-fold, or 19-fold, or 20-fold, or 21-fold, or 22-fold, or 23-fold, or 24-fold, or 25-fold, or more.

As discussed herein, the oncolytic virus of the invention is capable of expressing a first antibody molecule that specifically binds to CTLA-4. Cytotoxic T lymphocyte-associated antigen (CTLA-4 or CTLA4), also known as CD152, is a B7/CD28 family member that blocks T cell activation. CTLA-4 is expressed on activated T cells and transmits an inhibitory signal to T cells. It is homologous to the T cell co-stimulatory protein CD28, and both CTLA-4 and CD28 bind to CD80 (also denoted B7-1) and CD86 (also denoted B7-2). CTLA4 is also found in regulatory T cells (Tregs) and contributes to its inhibitory function. The CTLA-4 protein contains an extracellular V domain, a transmembrane domain, and a cytoplasmic tail.

Antibodies that bind CTLA-4 have been proposed to exert their therapeutic activity by dual mechanisms, acting both on immune effector CD4⁺ and CD8⁺ T cells and on immune suppressive T regulatory (Treg) cells. On effector T cells, antibodies to CTLA-4 that block the interaction of CTLA-4 with its ligands B7.1 and B7.2 can enhance immune responses and have been shown to be capable of stimulating potent anti-tumor immunity (Korman et al 2006, Checkpoint blockade in cancer immunotherapy, Adv Immunol. 90:297-339).

More recently, Fc effector function and Treg depletion were shown to contribute to and correlate with therapeutic activity of anti-CTLA-4 antibodies, including clinically relevant antibodies ipilimumab and tremelimumab (Arce Vargas, Furness et al. 2018). Efficacy and toxicity, the latter of which may be severe and of autoimmune nature, are thought to be linked in currently available systemic anti-CTLA-4 regimens. Approaches to deliver highly effective yet safe anti-CTLA-4 based ICB have accordingly been lacking. The inventors recently demonstrated that intratumorally delivered oncolytic viruses encoding Treg-depleting anti-CTLA-4 antibodies (i.t. vectorized anti-CTLA-4) had broad antitumor activity. Herein, the inventors unexpectedly demonstrate that i.t. vectorized anti-CTLA-4, in the context of PD-1/PD-L1 ICB, has efficacy against poorly immune infiltrated “cold” tumors, which were resistant to systemic antibody-mediated ICB. Further, owing to the tumor-restricted anti-CTLA-4 exposure associated with this approach, i.t. vectorized anti-CTLA-4 is indicated to be safe and well-tolerated compared with approved anti-CTLA-4 regimens.

As discussed herein, the invention also involves a second antibody molecule that specifically binds PD-1 and/or that specifically binds to PD-L1. In some embodiments, the second antibody molecule specifically binds to PD-1; in some embodiments, the second antibody molecule specifically binds to PD-L1; and in some embodiments, the second antibody molecule specifically binds to both PD-1 and PD-L1.

Programmed cell death protein 1 (PD-1 or PD1), also known as CD279, is found on the surface of T and B cells and suppresses T cell activity. PD-1 binds two ligands: PD-L1 and PD-L2. Programmed death-ligand 1 (PD-L1), also known as CD274, binds to its receptor PD-1 to produce an inhibitory signal which reduces the proliferation of T cells.

Antibodies are well known to those skilled in the art of immunology and molecular biology. Typically, an antibody comprises two heavy (H) chains and two light (L) chains. Herein, we sometimes refer to this complete antibody molecule as a full-size or full-length antibody. The antibody's heavy chain comprises one variable domain (VH) and three constant domains (CH1, CH2 and CH3), and the antibody's molecule light chain comprises one variable domain (VL) and one constant domain (CL). The variable domains (sometimes collectively referred to as the Fv region) bind to the antibody's target, or antigen. Each variable domain comprises three loops, referred to as complementary determining regions (CDRs), which are responsible for target binding. The constant domains are not involved directly in binding an antibody to an antigen, but exhibit various effector functions. Depending on the amino acid sequence of the constant region of their heavy chains, antibodies or immunoglobulins can be assigned to different classes. There are five major classes of immunoglobulins: IgA, IgD, IgE, IgG and IgM, and in humans several of these are further divided into subclasses (isotypes), e.g., IgG1, IgG2, IgG3, and IgG4; IgA1 and IgA2. Another part of an antibody is the Fc domain (otherwise known as the fragment crystallisable domain), which comprises two of the constant domains of each of the antibody's heavy chains. The Fc domain is responsible for interactions between the antibody and Fc receptor.

Fc receptors are membrane proteins which are often found on the cell surface of cells of the immune system (i.e. Fc receptors are found on the target cell membrane—otherwise known as the plasma membrane or cytoplasmic membrane). The role of Fc receptors is to bind antibodies via the Fc domain, and to internalize the antibody into the cell. In the immune system, this can result in antibody-mediated phagocytosis and antibody-dependent cell-mediated cytotoxicity.

A subgroup of the Fc receptors are Fcγ receptors (Fc-gamma receptors, FcgammaR), which are specific for IgG antibodies. There are two types of Fcγ receptors: activating Fcγ receptors (also denoted activatory Fcγ receptors) and inhibitory Fcγ receptors. The activating and the inhibitory receptors transmit their signals via immunoreceptor tyrosine-based activation motifs (ITAM) or immunoreceptor tyrosine-based inhibitory motifs (ITIM), respectively. In humans, FcγRIIb (CD32b) is an inhibitory Fcγ receptor, while FcγRI (CD64), FcγRIIa (CD32a), FcγRIIc (CD32c), FcγRIIIa (CD16a) and FcγRIV are activating Fcγ receptors. FcγRIIIb is a GPI-linked receptor expressed on neutrophils that lacks an ITAM motif but through its ability to cross-link lipid rafts and engage with other receptors is also considered activatory. In mice, the activating receptors are FcγRI, FcγRIII and FcγRIV.

It is well-known that antibodies modulate immune cell activity through interaction with Fcγ receptors. Specifically, how antibody immune complexes modulate immune cell activation is determined by their relative engagement of activating and inhibitory Fcγ receptors. Different antibody isotypes bind with different affinity to activating and inhibitory Fcγ receptors, resulting in different A:I ratios (activation:inhibition ratios) (Nimmerjahn et al; Science. 2005 Dec. 2; 310 (5753): 1510-2).

By binding to an inhibitory Fcγ receptor, an antibody can inhibit, block and/or downmodulate effector cell functions.

By binding to an activatory Fcγ receptor, an antibody can activate effector cell functions and thereby trigger mechanisms such as antibody-dependent cellular cytotoxicity (ADCC), antibody dependent cellular phagocytosis (ADCP), cytokine release, and/or antibody dependent endocytosis, as well as NETosis (i.e. activation and release of NETs, Neutrophil extracellular traps) in the case of neutrophils. Antibody binding to an activating Fcγ receptor can also lead to an increase in certain activation markers, such as CD40, MHCII, CD38, CD80 and/or CD86.

In some embodiments, the antibody molecule that specifically binds to CTLA-4 is an Fcγ receptor engaging antibody. By “Fcγ receptor engaging antibody” we mean that the antibody molecule can bind to at least one Fcγ receptor via its Fc region.

The term antibody molecule, as used herein, encompasses full-length or full-size antibodies as well as functional fragments of full-length antibodies and derivatives of such antibody molecules.

Functional fragments of a full-size antibody have the same antigen binding characteristics as the corresponding full-size antibody and include either the same variable domains (i.e. the VH and VL sequences) and/or the same CDR sequences as the corresponding full-size antibody. That the functional fragment has the same antigen binding characteristics as the corresponding full-size antibody means that it binds to the same epitope on the target as the full-size antibody. Such a functional fragment may correspond to the Fv part of a full-size antibody. Alternatively, such a fragment may be a Fab, also denoted Fab′, which is a monovalent antigen-binding fragment that does not contain a Fc part, or a (Fab′)₂, which is a divalent antigen-binding fragment that contains two antigen-binding Fab parts linked together by disulfide bonds or a Fab′, i.e. a monovalent-variant of a (Fab′)₂. Such a fragment may also be single chain variable fragment (scFv).

In some embodiments, the first antibody molecule and/or the second antibody molecule described herein is selected from the group consisting of a full-size antibody, a chimeric antibody, a single chain antibody, and an antigen-binding fragment thereof (e.g. a Fab, a Fv, an scFv, a Fab′, and a (Fab′)₂).

A functional fragment does not always contain all six CDRs of a corresponding full-size antibody. It is appreciated that molecules containing three or fewer CDR regions (in some cases, even just a single CDR or a part thereof) are capable of retaining the antigen-binding activity of the antibody from which the CDR(s) are derived. For example, in Gao et al., 1994, J. Biol. Chem., 269:32389-93 it is described that a whole VL chain (including all three CDRs) has a high affinity for its substrate.

Molecules containing two CDR regions have been described, for example, by Vaughan & Sollazzo 2001, Combinatorial Chemistry & High Throughput Screening, 4:417-430. On page 418 (right column—3 Our Strategy for Design) a minibody including only the H1 and H2 CDR hypervariable regions interspersed within framework regions is described. The minibody is described as being capable of binding to a target. Pessi et al., 1993, Nature, 362:367-9 and Bianchi et al., 1994, J. Mol. Biol., 236:649-59 are referenced by Vaughan & Sollazzo and describe the H1 and H2 minibody and its properties in more detail. In Qiu et al., 2007, Nature Biotechnology, 25:921-9 it is demonstrated that a molecule consisting of two linked CDRs are capable of binding antigen. Quiocho 1993, Nature, 362:293-4 provides a summary of “minibody” technology. Ladner 2007, Nature Biotechnology, 25:875-7 comments that molecules containing two CDRs are capable of retaining antigen-binding activity.

Antibody molecules containing a single CDR region are described, for example, in Laune et al., 1997, JBC, 272:30937-44, in which it is demonstrated that a range of hexapeptides derived from a CDR display antigen-binding activity and it is noted that synthetic peptides of a complete, single, CDR display strong binding activity. In Monnet et al., 1999, JBC, 274:3789-96 it is shown that a range of 12-mer peptides and associated framework regions have antigen-binding activity and it is commented on that a CDR3-like peptide alone is capable of binding antigen. In Heap et al., 2005, J. Gen. Virol., 86:1791-1800 it is reported that a “micro-antibody” (a molecule containing a single CDR) is capable of binding antigen and it is shown that a cyclic peptide from an anti-HIV antibody has antigen-binding activity and function. In Nicaise et al., 2004, Protein Science, 13:1882-91 it is shown that a single CDR can confer antigen-binding activity and affinity for its lysozyme antigen.

Thus, antibody molecules having five, four, three or fewer CDRs are capable of retaining the antigen binding properties of the full-length antibodies from which they are derived.

The antibody molecule may also be a derivative of a full-length antibody or a fragment of such an antibody. The derivative has the same antigen binding characteristics as the corresponding full-size antibody in the sense that it binds to the same epitope on the target as the full-size antibody.

Thus, by the term “antibody molecule”, as used herein, we include all types of antibody molecules and functional fragments thereof and derivatives thereof, including: monoclonal antibodies, polyclonal antibodies, synthetic antibodies, recombinantly produced antibodies, multi-specific antibodies, bi-specific antibodies, human antibodies, humanized antibodies, chimeric antibodies, single chain antibodies, variable fragments (Fvs), single-chain variable fragments (scFv fragments) including divalent single-chain variable fragments (di-scFvs) and disulfide-linked variable fragments, Fab fragments, F(ab′)₂ fragments, Fab′ fragments, antibody heavy chains, antibody light chains, homo-dimers of antibody heavy chains, homo-dimers of antibody light chains, heterodimers of antibody heavy chains, heterodimers of antibody light chains, antigen binding functional fragments of such homo- and heterodimers.

Further, the term “antibody molecule”, as used herein, includes all classes of antibody molecules and functional fragments, including: IgG, IgG1, IgG2, IgG3, IgG4, IgA, IgM, IgD, and IgE.

In some embodiments, the antibody is a human IgG1. The skilled person is aware that the mouse IgG2a and human IgG1 productively engage with activatory Fc gamma receptors and share the ability to activate deletion of target cells through activation of activatory Fc gamma receptor bearing immune cells (e.g. macrophages and NK cells) by e.g. ADCP and ADCC. As such, whereas the mouse IgG2a is the preferred isotype for deletion in the mouse, human IgG1 is a preferred isotype for deletion in human. Conversely, it is known that optimal co-stimulation of TNFR superfamily agonist receptors e.g. 4-1BB, OX40, TNFRII, CD40 depends on antibody engagement of the inhibitory FcγRIIB. In the mouse the IgG1 isotype, which binds preferentially to inhibitory Fc gamma receptor (FcγRIIB) and only weakly to activatory Fc gamma receptors, is known to be optimal for costimulatory activity of TNFR-superfamily targeting mAb. While no direct equivalent of the mouse IgG1 isotype has been described in man, antibodies may be engineered to show a similarly enhanced binding to inhibitory over activatory human Fc gamma receptors. Such engineered TNFR-superfamily targeting antibodies also have improved co-stimulatory activity in vivo, in transgenic mice engineered to express human activatory and inhibitory Fc gamma receptors (Dahan et al, 2016, Therapeutic Activity of Agonistic, Human Anti-CD40 Monoclonal Antibodies Requires Selective FcγR Engagement. Cancer Cell. 29 (6): 820-31).

As outlined above, different types and forms of antibody molecules are included in the invention, and would be known to the person skilled in immunology. It is well known that antibodies used for therapeutic purposes are often modified with additional components which modify the properties of the antibody molecule.

Accordingly, we include that an antibody molecule of the invention or an antibody molecule used in accordance with the invention (for example, a monoclonal antibody molecule, and/or polyclonal antibody molecule, and/or bi-specific antibody molecule) comprises a detectable moiety and/or a cytotoxic moiety.

By “detectable moiety”, we include one or more from the group comprising of: an enzyme; a radioactive atom; a fluorescent moiety; a chemiluminescent moiety; a bioluminescent moiety. The detectable moiety allows the antibody molecule to be visualised in vitro, and/or in vivo, and/or ex vivo.

By “cytotoxic moiety”, we include a radioactive moiety, and/or enzyme, for example wherein the enzyme is a caspase, and/or toxin, for example wherein the toxin is a bacterial toxin or a venom; wherein the cytotoxic moiety is capable of inducing cell lysis.

We further include that the antibody molecule may be in an isolated form and/or purified form, and/or may be PEGylated.

As discussed above, the CDRs of an antibody bind to the antibody target. The assignment of amino acids to each CDR described herein is in accordance with the definitions according to Kabat E A et al. 1991, In “Sequences of Proteins of Immunological Interest” Fifth Edition, NIH Publication No. 91-3242, pp xv-xvii.

As the skilled person would be aware, other methods also exist for assigning amino acids to each CDR. For example, the International ImMunoGeneTics information system (IMGT®) (http://www.imgt.org/and Lefranc and Lefranc “The Immunoglobulin FactsBook” published by Academic Press, 2001).

In a further embodiment, the CTLA-4 specific antibody molecules of the present invention or used according to the invention is an antibody molecule that is capable of competing with the specific antibodies described herein, such as the antibody molecules comprising SEQ ID. NOs: 15, 16, 17, 10, 18 and 19 or SEQ ID. NOs: 22, 23, 24, 10, 25 and 26.

By “capable of competing for” we mean that the competing antibody is capable of inhibiting or otherwise interfering, at least in part, with the binding of an antibody molecule as defined herein to the specific target.

For example, such a competing antibody molecule may be capable of inhibiting the binding of an antibody molecule described herein by at least about 10%; for example at least about 20%, or at least about 30%, at least about 40%, at least about 50%, at least about 60%, at least about 70%, at least about 80%, at least about 90%, at least about 95%, at least about 100% and/or inhibiting the ability of the antibody described herein to prevent or reduce binding to the specific target by at least about 10%; for example at least about 20%, at least about 30%, at least about 40%, at least about 50%, at least about 60%, at least about 70%, at least about 80%, at least about 90%, at least about 95%, or at least about 100%.

Competitive binding may be determined by methods well known to those skilled in the art, such as Enzyme-linked immunosorbent assay (ELISA).

ELISA assays can be used to evaluate epitope-modifying or blocking antibodies. Additional methods suitable for identifying competing antibodies are disclosed in Antibodies: A Laboratory Manual, Harlow & Lane, which is incorporated herein by reference (for example, see pages 567 to 569, 574 to 576, 583 and 590 to 612, 1988, CSHL, NY, ISBN 0-87969-314-2).

It is well known that an antibody specifically binds a defined target molecule or antigen, and that this means that the antibody preferentially and selectively binds its target and not a molecule which is not a target.

The targets of the first and second antibodies (CTLA-4, PD1, PD-L1) according to the present invention, or of the first and second antibodies used in accordance with the invention, is expressed on the surface of cells, i.e. they are cell surface antigen, which would include an epitope (otherwise known in this context as a cell surface epitope) for the antibody. Cell surface antigen and epitope are terms that would be readily understood by one skilled in immunology or cell biology.

By “cell surface antigen”, we include that the cell surface antigen or at least the epitope thereof to which the antibody molecule described herein, is exposed on the extracellular side of the cell membrane.

Methods of assessing protein binding are known to the person skilled in biochemistry and immunology. It would be appreciated by the skilled person that those methods could be used to assess binding of an antibody to a target and/or binding of the Fc domain of an antibody to an Fc receptor; as well as the relative strength, or the specificity, or the inhibition, or prevention, or reduction in those interactions. Examples of methods that may be used to assess protein binding are, for example, immunoassays, BIAcore, western blots, radioimmunoassay (RIA) and enzyme-linked immunosorbent assays (ELISAs) (See Fundamental Immunology Second Edition, Raven Press, New York at pages 332-336 (1989) for a discussion regarding antibody specificity).

Accordingly, herein both an “antibody molecule that specifically binds to CTLA-4” and an “anti-CTLA-4 antibody molecule” refers to an antibody molecule that specifically binds the target CTLA-4 but does not bind to non-target, or binds to a non-target more weakly (such as with a lower affinity) than the target.

Similarly, herein both an “antibody molecule that specifically binds to PD-1” and an “anti-PD-1 antibody molecule” refers to an antibody molecule that specifically binds to the target PD-1 but does not bind to non-target, or binds to a non-target more weakly (such as with a lower affinity) than the target.

Herein both an “antibody molecule that specifically binds to PD-L1” and an “anti-PD-L1 antibody molecule” refers to an antibody molecule that specifically binds to the target PD-L1 but does not bind to non-target, or binds to a non-target more weakly (such as with a lower affinity) than the target.

In some embodiments, the antibody molecule that specifically binds CTLA-4 (or the anti-CTLA-4 antibody molecule) refers to an antibody molecule that specifically binds to the extracellular domain of CTLA-4. In some embodiments, the antibody molecule that specifically binds PD-1 (or the anti-PD-1 antibody molecule) refers to an antibody molecule that specifically binds to the extracellular domain of PD-1. In some embodiments, the antibody molecule that specifically binds PD-L1 (or the anti-PD-L1 antibody molecule) refers to an antibody molecule that specifically binds to the extracellular domain of PD-L1.

We also include the meaning that the antibody specifically binds to the target CTLA-4 or PD-1 or PD-L1 at least two-fold more strongly, or at least five-fold more strongly, or at least 10-fold more strongly, or at least 20-fold more strongly, or at least 50-fold more strongly, or at least 100-fold more strongly, or at least 200-fold more strongly, or at least 500-fold more strongly, or at least than about 1000-fold more strongly than to a non-target.

Additionally, we include the meaning that the antibody specifically binds to the target CTLA-4 or PD-1 or PD-L1 if it binds to the target with a dissociation constant (K_{D}) of at least about 10⁻¹ M, or at least about 10⁻² M, or at least about 10⁻³ M, or at least about 10⁻⁴ M, or at least about 10⁻⁵ M, or at least about 10⁻⁶ M, or at least about 10⁻⁷ M, or at least about 10⁻⁸ M, or at least about 10⁻⁹ M, or at least about 10⁻¹⁰ M, or at least about 10⁻¹¹ M, or at least about 10⁻¹² M, or at least about 10⁻¹³ M, or at least about 10⁻¹⁴ M, or at least about 10⁻¹⁵ M.

As discussed above, the oncolytic virus of the invention expresses an antibody that specifically binds CTLA-4.

In some embodiments, the antibody molecule that specifically binds to CTLA-4 is an Fcγ receptor engaging antibody.

In some embodiments, the first antibody molecule is selected from the group consisting of ipilimumab and tremelimumab.

In some embodiments, the antibody molecule that specifically binds CTLA-4 (or the anti-CTLA-4 antibody molecule) does not cross react with CD28. In some embodiments, the antibody molecule that specifically binds CTLA-4 (or the anti-CTLA-4 antibody molecule) blocks the binding of CTLA-4 to CD80 and/or CD86, thereby inhibiting CTLA-4 signalling.

In some embodiments, the antibody molecules that specifically bind to CTLA-4 (or the anti-CTLA-4 antibody molecules) described herein has an improved depleting effect on CTLA-4 positive cells compared to ipilimumab.

That the antibody molecules have a depleting effect on CTLA-4 positive cells means that upon administration to a subject, such as a human, such an antibody binds specifically to CTLA-4 expressed on the surface of CTLA-4 positive cells, and this binding results in depletion of such cells.

In some embodiments, the CTLA-4 positive cells are CD4 positive (CD4+) cells, i.e. cells that express CD4.

In some embodiments, the CTLA-4 positive cells are both CD4 positive and FOXP3 positive, i.e. expressing both CD4 and FOXP3. These cells are Tregs. CD8 positive T cells also express CTLA-4, but Tregs express significantly higher levels of CTLA-4 than CD8 positive T cells. This makes Tregs more susceptible to depletion compared to lower expressing CD8+ cells.

In some situations, the CTLA-4 is preferentially expressed on immune cells in the tumor microenvironment (tumor infiltrating cells, TILS).

Thus, in a tumor microenvironment, the Tregs will be the cells that have the highest expression of CTLA-4, resulting in the antibody molecules that specifically bind to CTLA-4 (or the anti-CTLA-4 antibody molecules) having a Treg depleting effect.

Therefore, in some embodiments, the antibody molecule that specifically binds to CTLA-4 is an Treg depleting antibody.

As mentioned above, the anti-CTLA-4 antibody molecules described herein may be Treg depleting antibody molecules, which means that upon administration to a subject, such as a human, such an antibody molecule binds specifically to CTLA-4 expressed on the surface of Tregs, and this binding results in depletion of Tregs.

To decide whether an antibody molecule is an antibody molecule that has a Treg depleting effect as referred to herein (for example this may be an improved depleting effect compared to ipilimumab), it is possible to use an in vitro antibody-dependent cellular cytotoxicity (ADCC) assay or an in vivo test in a PBMC-NOG/SCID model.

The in vitro ADCC test which is performed using an NK-92 cell line stably transfected to express the CD16-158V allele together with GFP, wherein the ADCC test comprises the following consecutive seven steps:
1) CTLA-4 positive cells, CD4 positive cells or Tregs as target cells are isolated from peripheral blood of healthy donors. This isolation may be done using a CD4⁺ T cell isolation kit, such as a commercial kit from Miltenyi Biotec.
2) The target cells are then stimulated, e.g. for 48 hours, with CD3/CD28, for example using CD3/CD28 Dynabeads® and rhIL-2, such as 50 ng/ml rhIL-2. The stimulation may be done at 37° C.
3) The target cells are then pre-incubated with the antibody molecule to be tested, e.g. at 10 μg/ml for 30 min at 4° C., and are then mixed with NK cells.
4) The target cells are then incubated for an appropriate time, such as 4 hours, in RPMI 1640+GlutaMAX medium containing HEPES buffer, sodium pyruvate and FBS low IgG. The RPMI 1640+GlutaMAX medium may containing 10 mM HEPES buffer, 1 mM sodium pyruvate and 10% FBS low IgG, and the effector:target cell ratio may be 2:1.
5) Lysis is determined by flow cytometry.
6) Steps 1-5 are repeated, or performed in parallel, with ipilimumab (as a control) used instead of the tested antibody molecule in step 3.
7) The results of the lysis for the tested antibody molecule are compared to the results of the lysis for ipilimumab. An improved lysis for the tested antibody molecule compared to ipilimumab demonstrates that the tested antibody molecule has improved depleting effect on CTLA-4 positive cells, CD4 positive cells or Tregs, respectively, depending on which target cells were used.

In some embodiments, the improved depleting effect in step 7) above is a significantly improved depleting effect.

The in vivo test is based on the combined use of PBMC mice and NOG/SCID mice, which is herein called a PBMC-NOG/SCID model. Both PBMC mice and NOG/SCID mice are well-known models. The in vivo test in the PBMC-NOG/SCID model comprises the following consecutive nine steps:
1) Human PBMCs (peripheral blood mononuclear cells) are isolated, washed and resuspended in sterile PBS. In some embodiments, the PBMCs are resuspended in PBS at 75×10⁶ cells/ml.
2) NOG mice are injected i.v. (intravenously) with an appropriate amount, such as 200 μl, of the cell suspension from step 1). If 200 μl are injected, this corresponds to 15×10⁶ cells/mouse.
3) A suitable time, such as 2 weeks, after injection, the spleens from the NOG mice are isolated and rendered into a single cell suspension. Optionally, a small sample from the single cell suspension is taken to determine the expression of CTLA-4 on human T cells by FACS, in order to confirm the CTLA-4 expression.
4) The cell suspension from step 3) is resuspended in sterile PBS. In some embodiments, the cell suspension is resuspended in sterile PBS at 50×10⁶ cells/ml. If the optional CTLA-4 expression determination is included in step 3, the rest of the cell suspension is then resuspended in step 4.
5) SCID mice are injected i.p. (intraperitoneally) with an appropriate amount, such as 200 μl, of the suspension from step 4. If 200 μl are injected, this corresponds to 10×10⁶ cells/mouse.
6) A suitable time, such as 1 hour, after the injection in step 5) the SCID mice are treated with an appropriate amount, such as 10 mg/kg, of either the antibody molecule to be tested, ipilimumab or an isotype control monoclonal antibody.
7) The intraperitoneal fluid of the treated SCID mice is collected a suitable time, such as 24 hours, after the treatment in step 6).
8) Human T cell subsets are identified and quantified by FACS using following markers: CD45, CD4, CD8, CD25 and/or CD127.
9) The results from identification and quantification of the T cell subsets from the mice treated with the tested antibody molecule is compared to the results from identification and quantification of the T cell subsets from the mice treated with ipilimumab and to the results from identification and quantification of the T cell subsets from the mice treated with isotype control monoclonal antibody. A lower number of CTLA-4 positive cells in the intraperitoneal fluid from mice treated with the antibody molecule to be tested compared to the number of CTLA-4 positive cells in the intraperitoneal fluid from mice treated with ipilimumab demonstrates that the antibody molecule has improved depleting effect on CTLA-4 positive cells compared to ipilimumab. A lower number of CD4 positive cells in the intraperitoneal fluid from mice treated with the antibody molecule to be tested compared to the number of CD4 positive cells in the intraperitoneal fluid from mice treated with ipilimumab demonstrates that the antibody molecule has improved depleting effect on CD4 positive cells compared to ipilimumab. A lower number of Tregs in the intraperitoneal fluid from mice treated with the antibody molecule to be tested compared to the number of Tregs in the intraperitoneal fluid from mice treated with ipilimumab demonstrates that the antibody molecule has improved depleting effect on Tregs compared to ipilimumab.

In this in vivo test, it is in some embodiments of most interest to look at the Treg depletion in step 7.

Treg depletion may also be assessed in an antibody-dependent cellular phagocytosis (ADCP) assay, as known to the skilled person.

In some embodiments, the antibody molecules have similar blocking effect on CTLA-4 interactions with B7.1 and B7.2 ligands compared to Yervoy (ipilimumab). This may be assessed by ELISA or in a more functional assay where anti-CTLA-4 antibodies enhance the IL-2 production by T cells in response to stimulation of PBMCs with Staphylococcus Enterotoxin B (SEB).

In some embodiments, the anti-CTLA-4 antibody molecule is a human antibody molecule. In some embodiments, the anti-CTLA-4 antibody molecule is a humanized antibody molecule. In some embodiments, the anti-CTLA-4 antibody molecule is an antibody molecule of human origin, meaning that it originates from a human antibody molecule which then has been modified. In some embodiments, the anti-CTLA-4 antibody molecule is a human IgG1 antibody.

In some embodiments, the first antibody molecule that specifically binds to CTLA-4 is selected from the group consisting of a human IgG antibody, a humanized IgG antibody and an IgG antibody of human origin.

In some embodiments, the first antibody molecule that specifically binds to CTLA-4 is selected from the group consisting of a full-size antibody, a chimeric antibody, a single chain antibody, and an antigen-binding fragment thereof (e.g. a Fab, a Fv, an scFv, a Fab′, and a (Fab′)₂).

In some embodiments, the anti-CTLA-4 antibody is an antibody in the form of a human IgG1 antibody showing improved binding to one or several activatory Fc receptors and/or being engineered for improved binding to one or several activatory Fc receptors; accordingly, in some embodiments, the anti-CTLA-4 antibody is an Fc-engineered human IgG1 antibody.

In some embodiments, the anti-CTLA-4 antibody is a murine or a humanized murine IgG2a antibody.

In some embodiments, the anti-CTLA-4 antibody is a murine antibody that is cross-reactive with human CTLA-4.

In some embodiments, the anti-CTLA-4 antibody is a monoclonal antibody or an antibody molecule of monoclonal origin. In some embodiments, the anti-CTLA-4 antibody is a polyclonal antibody.

In some embodiments, the anti-CTLA-4 antibody molecule is an antibody molecule comprising one of the three alternative VH-CDR1 sequences, one of the three alternative VH-CDR2 sequences, one of the two alternative VH-CDR3 sequences, one of the two VL-CDR1 sequences, one of the two VL-CDR2 sequences, and/or one of the two alternative VL-CDR3 sequences presented in Table 1 below.

In some embodiments, the anti-CTLA-4 antibody molecule is selected from the group consisting of antibody molecules comprising 1-6 of the CDRs selected from the group consisting of SEQ ID. Nos: 3, 6, 8, 10, 12 and 14.

In some embodiments, the anti-CTLA-4 antibody molecule is selected from the group consisting of antibody molecules comprising the CDRs having SEQ ID. Nos: 3, 6, 8, 10, 12 and 14.

In some embodiments, the anti-CTLA-4 antibody molecule is selected from the group consisting of antibody molecules comprising 1-6 of the CDRs, VH-CDR1, VH-CDR2, VH-CDR3, VL-CDR1, and VL-CDR3,
wherein VH-CDR1, if present, is selected from the group consisting of SEQ ID. Nos: 15, 22, 29 and 35;
wherein VH-CDR2, if present, is selected from the group consisting of SEQ ID. Nos: 16, 23, 30, and 36;
wherein VH-CDR3, if present, is selected from the group consisting of SEQ ID. Nos: 17, 24, 31 and 37;
wherein VL-CDR1, if present, is selected from the group consisting of SEQ ID. Nos: 10 and 38;
wherein VL-CDR2, if present, is selected from the group consisting of SEQ ID. Nos: 18, 25, 32 and 39;
wherein VL-CDR3, if present, is selected from the group consisting of SEQ ID. Nos: 19, 26 and 40.

In some embodiments, the anti-CTLA-4 antibody molecule is an antibody molecule selected from the group consisting of antibody molecules comprising 6 CDRs selected from the group consisting of:
SEQ ID. NOS: 15, 16, 17, 10, 18 and 19;
SEQ ID. NOs: 22, 23, 24, 10, 25 and 26;
SEQ ID. NOs: 29, 30, 31, 10, 32 and 26; and
SEQ ID. NOs: 35, 36, 37, 38, 39 and 40.

In some embodiments, the anti-CTLA-4 antibody molecule is an antibody molecule comprising the 6 CDRs having SEQ ID. NOs: 15, 16, 17, 10, 18 and 19.

In some embodiments, the anti-CTLA-4 antibody molecule is an antibody molecule comprising the 6 CDRs having SEQ ID. NOs: 22, 23, 24, 10, 25 and 26.

In some embodiments, the anti-CTLA-4 antibody molecule is an antibody molecule selected from the group consisting of antibody molecules comprising a VH selected from the group consisting of SEQ ID. NOs: 20, 27, 33 and 41.

In some embodiments, the anti-CTLA-4 antibody molecule is an antibody molecule selected from the group consisting of antibody molecules comprising a VL selected from the group consisting of SEQ ID. NOs: 21, 28, 34 and 42.

In some embodiments, the anti-CTLA-4 antibody molecule is an antibody molecule selected from the group consisting of antibody molecules comprising a VH and a VL selected from the group consisting of: SEQ ID. Nos: 20-21, 27-28, 33-34 and 41-42.

In some embodiments, the anti-CTLA-4 antibody molecule comprises a VH having sequence SEQ ID. No: 20 and a VL having sequence SEQ ID. No: 21.

In some embodiments, the anti-CTLA-4 antibody molecule comprises a VH having sequence SEQ ID. No: 27 and a VL having sequence SEQ ID. No: 28.

In some embodiment, the first antibody molecule comprises a variable heavy chain selected from the group consisting of SEQ. ID. NOs: 20 and 27. In some additional or alternative embodiments, the first antibody molecule comprises a variable light chain selected from the group consisting of SEQ ID NOS: 21 and 28.

In some embodiments, the anti-CTLA-4 antibody molecule comprises a heavy chain constant region (CH) having the sequence SEQ ID NO: 43. In some additional or alternative embodiments, the anti-CTLA-4 antibody molecule comprises a light chain constant region (CL) having the sequence SEQ ID NO: 44. In some embodiment, the anti-CTLA-4 antibody molecule comprises a constant region of SEQ ID NOs: 43 and 44.

TABLE 1
General CDR sequences of antibodies disclosed herein

| Relevant | | Explanation of | |
|---|---|---|---|
| part of | | unidentified amino | SEQ. |
| antibody | Sequence | acid residues | ID. NO |
| VH-CDR1 | FX₁X₂YX₃MX₄WX₅RQAPG | X₁ = S or K; X₂ = D, S | 1 |
| alternative | | or A; X₃ = Y, S or A; | |
| 1 | | X₄ = S or N; and X₅ = | |
| | | V or I | |
| VH-CDR1 | FSX₁YX₂MX₃WVRQAPG | X₁ = D or S; X₂ = Y, S | 2 |
| alternative | | or A; and X₃ = S or N | |
| 2 | | | |
| VH-CDR1 | FSX₁YX₂MX₃WVRQAPG | X₁ = D or S; X₂ = Y or | 3 |
| alternative | | S; and X₃ = S or N | |
| 3 | | | |
| VH-CDR2, | SX₁ISX₂X₃X₄X₅X₆X₇X₈X₉ADSVKGR | X₁ = G or A; X₂ = W, | 4 |
| alternative | | G or N; X₃ = S or T; | |
| 1 | | X₄ = S or G; X₅ = R or | |
| | | G; X₆ = D, S or Y; X₇ = | |
| | | K, T or I; X₈ = G, Y, | |
| | | H or D; X₉ = Y or F | |
| VH-CDR2, | SX₁ISX₂X₃X₄X₅X₆X₇X₈YADSVKGR | X₁ = G or A; X₂ = W, | 5 |
| alternative | | G or N; X₃ = S or T; | |
| 2 | | X₄ = S or G; X₅ = R or | |
| | | G; X₆ = D, S or Y; X₇ = | |
| | | K, T or I; X₈ = G, Y, | |
| | | H or D | |
| VH-CDR2, | SX₁ISX₂SX₃X₄X₅X₆X₇YADSVKGR | X₁ = G or A; X₂ = W | 6 |
| alternative | | or G; X₃ = S or G; X₄ = | |
| 3 | | R or G; X₅ = D or S; | |
| | | X₆ = K or T; X₇ = G or | |
| | | Y, H or D | |
| VH-CDR3 | X₁X₂X₃X₄X₅X₆X₇X₈X₉X₁₀X₁₁X₁₂X₁₃X₁₄X₁₅ | X₁ = T or A; X₂ = T or | 7 |
| alternative | | R; X₃ = D, Y or L; X₄ = | |
| 1 | | L, R, S, or G; X₅ = | |
| | | A, V, S or Y; X₆ = R, | |
| | | E, G or S; X₇ = Y, M, | |
| | | L or G; X₈ = N, H, Y or | |
| | | none; X₉ = Q, D or | |
| | | none; X₁₀ = W, A, D | |
| | | or none; X₁₁ = L, F, R | |
| | | or none; X₁₂ = A, D, G | |
| | | or none; X₁₃ = D, I, M | |
| | | or none; X₁₄ = D or | |
| | | none; and X₁₅ = V or | |
| | | none | |
| VH-CDR3 | X₁X₂DX₃X₄X₅X₆X₇X₈X₉X₁₀X₁₁X₁₂X₁₃ | X₁ = T or A; X₂ = T or | 8 |
| alternative | | R;; X₃ = L or R; X₄ = | |
| 2 | | A or V; X₅ = R or E; | |
| | | X₆ = Y or M; X₇ = N or | |
| | | none; X₈ = Q or none; | |
| | | X₉ = W or none; X₁₀ = | |
| | | L, or none; X₁₁ = A, or | |
| | | none; X₁₂ = D or none | |
| VL-CDR1 | CX₁GSSSNIGX₂X₃YX₄X₅X₆ | X₁ = T or S; X₂ = A or | 9 |
| alternative | | S; X₃ = G or N; X₄ = | |
| 1 | | D or V; X₅ = V or Y; | |
| | | X₆ = H or none | |
| VL-CDR1 | CTGSSSNIGAGYDVH | | 10 |
| alternative | | | |
| 2 | | | |
| VL-CDR2 | X₁NX₂X₃RPS | X₁ = G, R, or D; X₂ = | 11 |
| alternative | | D, N, or S; and X₃ = | |
| 1 | | N, Q or K | |
| VL-CDR2 | X₁NX₂X₃RPS | X₁ = G or R; X₂ = D or | 12 |
| alternative | | N; and X₃ = N or Q | |
| 2 | | | |
| VL-CDR3 | CX₁X₂X₃DX₄SLX₅GX₆VX₇ | X₁ = A or Q; X₂ = V, A | 13 |
| alternative | | or S; X₃ = W or Y; X4 = | |
| 1 | | D or S; X₅ = N or S; | |
| | | X₆ = V, W or P; and X₇ = | |
| | | V or none | |
| VL-CDR3 | CAX₁WDDSLNGX₂V | X₁ = V or A; and X₂ = | 14 |
| alternative | | V or W | |
| 2 | | | |

TABLE 2
Specific anti-CTLA-4 antibody molecules; the CDR sequences are marked in bold
in the full VH and VL sequences

| Antibody | | | SEQ. ID. |
|---|---|---|---|
| clone | Region | Sequence | NO: |
| 4-E03 | VH- | FSDYYMSWVR QAPG | 15 |
| | CDR1 | | |
| | VH- | SGISWSSRDK GYADSVKGR | 16 |
| | CDR2 | | |
| | VH- | TTDLARY | 17 |
| | CDR3 | | |
| | VL-CDR1 | CTGSSSNIGA GYDVH | 10 |
| | VL-CDR2 | GNDNRPS | 18 |
| | VL-CDR3 | CAVWDDSLNG VV | 19 |
| | VH | EVQLLESGGG LVQPGGSLRL SCAASGFTFS | 20 |
| | | DYYMSWVRQA PGKGLEWVSG ISWSSRDKGY | |
| | | ADSVKGRFTI SRDNSKNTLY LQMNSLRAED | |
| | | TAVYYCTTDL ARYWGQGTLV TVSS | |
| | VL | QSVLTQPPSA SGTPGQRVTI SCTGSSSNIG | 21 |
| | | AGYDVHWYQQ LPGTAPKLLI YGNDNRPSGV | |
| | | PDRFSGSKSG TSASLAISGL RSEDEADYYC | |
| | | AVWDDSLNGV VFGGGTKLTV LG | |
| | human | MGWSCIILFLVATATGVHSQSVLTQPPSASGTPGQRVT | 53 |
| | immune_ | ISCTGSSSNIGAGYDVHWYQQLPGTAPKLLIYGNDNRPSG | |
| | globulin | VPDRFSGSKSGTSASLAISGLRSEDEADYYCAVWDDSLN | |
| | G1 | GVVFGGGTKLTVLGQPKAAPSVTLFPPSSEELQANKATLVC | |
| | (IgG1) | LISDFYPGAVTVAWKADSSPVKAGVETTTPSKQSNNKYAA | |
| | light | SSYLSLTPEQWKSHRSYSCQVTHEGSTVEKTVAPTECS | |
| | chain | | |
| | (LC) | | |
| | human | MGWSCIILFLVATATGVHSEVQLLESGGGLVQPGGSLR | 54 |
| | IgG1 | LSCAASGFTFSDYYMSWVRQAPGKGLEWVSGISWSSRD | |
| | heavy | KGYADSVKGRFTISRDNSKNTLYLQMNSLRAEDTAVYYCT | |
| | chain | TDLARYWGQGTLVTVSSASTKGPSVFPLAPSSKSTSGGTA | |
| | (HC) | ALGCLVKDYFPEPVTVSWNSGALTSGVHTFPAVLQSSGLY | |
| | | SLSSVVTVPSSSLGTQTYICNVNHKPSNTKVDKKVEPKSC | |
| | | DKTHTCPPCPAPELLGGPSVFLFPPKPKDTLMISRTPEVTCV | |
| | | VVDVSHEDPEVKFNWYVDGVEVHNAKTKPREEQYNSTYR | |
| | | VVSVLTVLHQDWLNGKEYKCKVSNKALPAPIEKTISKAKG | |
| | | QPREPQVYTLPPSRDELTKNQVSLTCLVKGFYPSDIAVEWE | |
| | | SNGQPENNYKTTPPVLDSDGSFFLYSKLTVDKSRWQQGN | |
| | | VFSCSVMHEALHNHYTQKSLSLSPGK | |
| 5-B07 | VH- | FSSYSMNWVRQ APG | 22 |
| | CDR1 | | |
| | VH- | SAISGSGGST YYADSVKGR | 23 |
| | CDR2 | | |
| | VH- | ARDRVEMNQW LAD | 24 |
| | CDR3 | | |
| | VL-CDR1 | CTGSSSNIGA GYDVH | 10 |
| | VL-CDR2 | RNNQRPS | 25 |
| | VL-CDR3 | CAAWDDSLNG WV | 26 |
| | VH | EVQLLESGGG LVQPGGSLRL SCAASGFTFS | 27 |
| | | SYSMNWVRQA PGKGLEWVSA ISGSGGSTYY | |
| | | ADSVKGRFTI SRDNSKNTLY LQMNSLRAED | |
| | | TAVYYCARDR VEMNQWLADW GQGTLVTVSS | |
| | VL | QSVLTQPPSA SGTPGQRVTI SCTGSSSNIG | 28 |
| | | AGYDVHWYQQ LPGTAPKLLI YRNNQRPSGV | |
| | | PDRFSGSKSG TSASLAISGL RSEDEADYYC | |
| | | AAWDDSLNGW VFGGGTKLTV LG | |
| | murine | MGWSCIILFLVATATGVHSQSVLTQPPSASGTPGQRVTIS | 62 |
| | immuno | CTGSSSNIGAGYDVHWYQQLPGTAPKLLIYRNNQRPSGVP | |
| | globulin | DRFSGSKSGTSASLAISGLRSEDEADYYCAAWDDSLNGW | |
| | (IgG2a) | VFGGGTKLTVLGQPKSSPSVTLFPPSSEELETNKATLVCTIT | |
| | light | DFYPGVVTVDWKVDGTPVTQGMETTQPSKQSNNKYMAS | |
| | chain | SYLTLTARAWERHSSYSCQVTHEGHTVEKSLSRADCS | |
| | (LC) | | |
| | murine | MGWSCIILFLVATATGVHSEVQLLESGGGLVQPGGSLRLS | 63 |
| | immuno | CAASGFTFSSYSMNWVRQAPGKGLEWVSAISGSGGSTYY | |
| | globulin | ADSVKGRFTISRDNSKNTLYLQMNSLRAEDTAVYYCARDR | |
| | (IgG2a) | VEMNQWLADWGQGTLVTVSSAKTTAPSVYPLAPVCGDTT | |
| | heavy | GSSVTLGCLVKGYFPEPVTLTWNSGSLSSGVHTFPAVLQS | |
| | chain | DLYTLSSSVTVTSSTWPSQSITCNVAHPASSTKVDKKIEPR | |
| | (HC) | GPTIKPCPPCKCPAPNLLGGPSVFIFPPKIKDVLMISLSPIVT | |
| | | CVVVDVSEDDPDVQISWFVNNVEVHTAQTQTHREDYNST | |
| | | LRVVSALPIQHQDWMSGKEFKCKVNNKDLPAPIERTISKP | |
| | | KGSVRAPQVYVLPPPEEEMTKKQVTLTCMVTDFMPEDIYV | |
| | | EWTNNGKTELNYKNTEPVLDSDGSYFMYSKLRVEKKNWV | |
| | | ERNSYSCSVVHEGLHNHHTTKSFSRTPGK | |
| 2-C06 | VH- | FSSYAMSWVRQ APG | 29 |
| | CDR1 | | |
| | VH- | SGISGSGGYI HYADSVKGR | 30 |
| | CDR2 | | |
| | VH- | ATYSSGLHDA FDI | 31 |
| | CDR3 | | |
| | VL-CDR1 | CTGSSSNIGA GYDVH | 10 |
| | VL-CDR2 | DNNKRPS | 32 |
| | VL-CDR3 | CAAWDDSLNG WV | 26 |
| | VH | EVQLLESGGG LVQPGGSLRL SCAASGFTFS | 33 |
| | | SYAMSWVRQA PGKGLEWVSG ISGSGGYIHY | |
| | | ADSVKGRFTI SRDNSKNTLY LQMNSLRAED | |
| | | TAVYYCATYS SGLHDAFDIW GQGTLVTVSS | |
| | VL | QSVLTQPPSA SGTPGQRVTI SCTGSSSNIG | 34 |
| | | AGYDVHWYQQ LPGTAPKLLI YDNNKRPSGV | |
| | | PDRFSGSKSG TSASLAISGL RSEDEADYYC | |
| | | AAWDDSLNGW VFGGGTKLTV LG | |
| 2-F09 | VH- | FKAYSMSWIR QAPG | 35 |
| | CDR1 | | |
| | VH- | SGISNTGGST DFADSVKGR | 36 |
| | CDR2 | | |
| | VH- | ARLGYSGYDD RGMDV | 37 |
| | CDR3 | | |
| | VL-CDR1 | CSGSSSNIGS NYVY | 38 |
| | VL-CDR2 | GNSNRPS | 39 |
| | VL-CDR3 | CQSYDSSLSG PVV | 40 |
| | VH | EVQLLESGGG LVQPGGSLRL SCAASGFTFK | 41 |
| | | AYSMSWIRQA PGKGLEWVSG ISNTGGSTDF | |
| | | ADSVKGRFTI SRDNSKNTLY LQMNSLRAED | |
| | | TAMYYCARLG YSGYDDRGMD VWGQGTLVTV SS | |
| | VL | QSVLTQPPSA SGTPGQRVTI SCSGSSSNIG | 42 |
| | | SNYVYWYQQL PGTAPKLLIY GNSNRPSGVP | |
| | | DRFSGSKSGT SASLAISGLR SEDEADYYCQ | |
| | | SYDSSLSGPV VFGGGTKLTV LG | |

In some embodiments, the anti-CTLA-4 antibody molecules described herein may also comprise one or both of the constant regions presented in Table 3 below.

TABLE 3
Sequences of constant regions of antibodies disclosed herein

| | | SEQ. |
|---|---|---|
| | | ID. |
| Region | Sequence | NO: |
| CH | ASTKGPSVFPLAPSSKSTSGGTAALGCLVKDYFPEPVTVSWNSGALTS | 43 |
| | GVHTFPAVLQSSGLYSLSSVVTVPSSSLGTQTYICNVNHKPSNTKVDKK | |
| | VEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTK | |
| | NQVSLTCLVKGFYPSDIAVEWESNGQPENNYKTTPPVLDSDGSFFLYSK | |
| | LTVDKSRWQQGNVFSCSVMHEALHNHYTQKSLSLSPGK | |
| CL | QPKAAPSVTLFPPSSEELQANKATLVCLISDFYPGAVTVAWKADSSPVK | 44 |
| | AGVETTTPSKQSNNKYAASSYLSLTPEQWKSHRSYSCQVTHEGSTVEK | |
| | TVAPTECS | |

In some embodiments, the anti-CTLA-4 antibody molecule is a molecule encoded by one of the nucleotide sequences presented in Table 4 below.

TABLE 4
Specific nucleotide sequences encoding anti-CTLA-4 antibody molecules

| | | | SEQ. |
|---|---|---|---|
| | | | ID. |
| Clone | Encoding | Sequence | NO: |
| 4-E03 | 4-E03 | GAGGTGCAGCTGTTGGAGTCTGGGGGAGGCTTGGTACAG | 45 |
| γ1 | VH | CCTGGGGGGTCCCTGAGACTCTCCTGTGCAGCCTCTGGA | |
| heavy | | TTCACCTTCAGTGACTACTACATGAGCTGGGTCCGCCAGG | |
| chain | | CTCCCGGGAAGGGGCTGGAGTGGGTCTCAGGCATTAGTT | |
| | | GGAGTAGTCGTGACAAAGGCTATGCGGACTCTGTGAAGG | |
| | | GCCGTTTCACCATCTCCAGAGACAATTCCAAGAACACGCT | |
| | | GTATCTGCAAATGAACAGCCTGAGAGCCGAGGACACTGC | |
| | | CGTGTATTACTGTACCACAGATCTCGCTAGGTACTGGGGC | |
| | | CAGGGTACACTGGTCACCGTGAGCTCAGCCTCCACCAAG | |
| | | GGCCCATCGGTCTTCCCCCTGGCACCCTCCTCCAAGAGCA | |
| | | CCTCTGGGGGCACAGCGGCCCTGGGCTGCCTGGTCAAGG | |
| | | ACTACTTCCCCGAACCGGTGACGGTGTCGTGGAACTCAG | |
| | | GCGCCCTGACCAGCGGCGTGCACACCTTCCCGGCTGTCC | |
| | | TACAGTCCTCAGGACTCTACTCCCTCAGCAGCGTGGTGAC | |
| | | CGTGCCCTCCAGCAGCTTGGGCACCCAGACCTACATCTGC | |
| | | AACGTGAATCACAAGCCCAGCAACACCAAGGTGGACAAG | |
| | | AAAGTTGAGCCCAAATCTTGTGACAAAACTCACACATGCC | |
| | | CACCGTGCCCAGCACCTGAACTCCTGGGGGGACCGTCAG | |
| | | TCTTCCTCTTCCCCCCAAAACCCAAGGACACCCTCATGATC | |
| | | TCCCGGACCCCTGAGGTCACATGCGTGGTGGTGGACGTG | |
| | | AGCCACGAAGACCCTGAGGTCAAGTTCAACTGGTACGTG | |
| | | GACGGCGTGGAGGTGCATAATGCCAAGACAAAGCCGCGG | |
| | | GAGGAGCAGTACAACAGCACGTACCGTGTGGTCAGCGTC | |
| | | CTCACCGTCCTGCACCAGGACTGGCTGAATGGCAAGGAG | |
| | | TACAAGTGCAAGGTCTCCAACAAAGCCCTCCCAGCCCCCA | |
| | | TCGAGAAAACCATCTCCAAAGCCAAAGGGCAGCCCCGAG | |
| | | AACCACAGGTGTACACCCTGCCCCCATCCCGGGATGAGCT | |
| | | GACCAAGAACCAGGTCAGCCTGACCTGCCTGGTCAAAGG | |
| | | CTTCTATCCCAGCGACATCGCCGTGGAGTGGGAGAGCAA | |
| | | TGGGCAGCCGGAGAACAACTACAAGACCACGCCTCCCGT | |
| | | GCTGGACTCCGACGGCTCCTTCTTCCTCTACAGCAAGCTC | |
| | | ACCGTGGACAAGAGCAGGTGGCAGCAGGGGAACGTCTTC | |
| | | TCATGCTCCGTGATGCATGAGGCTCTGCACAACCACTACA | |
| | | CGCAGAAGAGCCTCTCCCTGTCTCCGGGTAAATGA | |
| 4-E03 λ | 4-E03 | CAGTCTGTGCTGACTCAGCCACCCTCAGCGTCTGGGACCC | 46 |
| light | VL | CCGGGCAGAGGGTCACCATCTCCTGCACTGGGAGCAGCT | |
| chain | | CCAACATCGGGGCAGGTTATGATGTACACTGGTATCAGCA | |
| | | GCTCCCAGGAACGGCCCCCAAACTCCTCATCTATGGTAAT | |
| | | GATAACCGGCCCTCAGGGGTCCCTGACCGATTCTCTGGCT | |
| | | CCAAGTCTGGCACCTCAGCCTCCCTGGCCATCAGTGGGCT | |
| | | CCGGTCCGAGGATGAGGCTGATTATTACTGTGCAGTATGG | |
| | | GATGACAGCCTGAATGGTGTGGTATTCGGCGGAGGAACC | |
| | | AAGCTGACGGTCCTAGGTCAGCCCAAGGCTGCCCCCTCG | |
| | | GTCACTCTGTTCCCGCCCTCCTCTGAGGAGCTTCAAGCCA | |
| | | ACAAGGCCACACTGGTGTGTCTCATAAGTGACTTCTACCC | |
| | | GGGAGCCGTGACAGTGGCCTGGAAGGCAGATAGCAGCCC | |
| | | CGTCAAGGCGGGAGTGGAGACCACCACACCCTCCAAACA | |
| | | AAGCAACAACAAGTACGCGGCCAGCAGCTATCTGAGCCT | |
| | | GACGCCTGAGCAGTGGAAGTCCCACAGAAGCTACAGCTG | |
| | | CCAGGTCACGCATGAAGGGAGCACCGTGGAGAAGACAGT | |
| | | GGCCCCTACAGAATGTTCATGA | |
| 5-B07 | 5-B07 | GAGGTGCAGCTGTTGGAGTCTGGGGGAGGCTTGGTACAG | 47 |
| γ1 | VH | CCTGGGGGGTCCCTGAGACTCTCCTGTGCAGCCTCTGGA | |
| heavy | | TTCACCTTCAGTAGCTATAGCATGAACTGGGTCCGCCAGG | |
| chain | | CTCCAGGGAAGGGGCTGGAGTGGGTCTCAGCTATTAGTG | |
| | | GTAGTGGTGGTAGCACATACTACGCAGACTCCGTGAAGG | |
| | | GCCGGTTCACCATCTCCAGAGACAATTCCAAGAACACGCT | |
| | | GTATCTGCAAATGAACAGCCTGAGAGCCGAGGACACTGC | |
| | | CGTGTATTACTGTGCGAGAGATCGGGTAGAGATGAACCA | |
| | | GTGGCTGGCCGACTGGGGCCAGGGTACACTGGTCACCGT | |
| | | GAGCTCAGCCTCCACCAAGGGCCCATCGGTCTTCCCCCTG | |
| | | GCACCCTCCTCCAAGAGCACCTCTGGGGGCACAGCGGCC | |
| | | CTGGGCTGCCTGGTCAAGGACTACTTCCCCGAACCGGTG | |
| | | ACGGTGTCGTGGAACTCAGGCGCCCTGACCAGCGGCGTG | |
| | | CACACCTTCCCGGCTGTCCTACAGTCCTCAGGACTCTACT | |
| | | CCCTCAGCAGCGTGGTGACCGTGCCCTCCAGCAGCTTGG | |
| | | GCACCCAGACCTACATCTGCAACGTGAATCACAAGCCCAG | |
| | | CAACACCAAGGTGGACAAGAAAGTTGAGCCCAAATCTTGT | |
| | | GACAAAACTCACACATGCCCACCGTGCCCAGCACCTGAAC | |
| | | TCCTGGGGGGACCGTCAGTCTTCCTCTTCCCCCCAAAACC | |
| | | CAAGGACACCCTCATGATCTCCCGGACCCCTGAGGTCACA | |
| | | TGCGTGGTGGTGGACGTGAGCCACGAAGACCCTGAGGTC | |
| | | AAGTTCAACTGGTACGTGGACGGCGTGGAGGTGCATAAT | |
| | | GCCAAGACAAAGCCGCGGGAGGAGCAGTACAACAGCACG | |
| | | TACCGTGTGGTCAGCGTCCTCACCGTCCTGCACCAGGACT | |
| | | GGCTGAATGGCAAGGAGTACAAGTGCAAGGTCTCCAACA | |
| | | AAGCCCTCCCAGCCCCCATCGAGAAAACCATCTCCAAAGC | |
| | | CAAAGGGCAGCCCCGAGAACCACAGGTGTACACCCTGCC | |
| | | CCCATCCCGGGATGAGCTGACCAAGAACCAGGTCAGCCT | |
| | | GACCTGCCTGGTCAAAGGCTTCTATCCCAGCGACATCGCC | |
| | | GTGGAGTGGGAGAGCAATGGGCAGCCGGAGAACAACTAC | |
| | | AAGACCACGCCTCCCGTGCTGGACTCCGACGGCTCCTTCT | |
| | | TCCTCTACAGCAAGCTCACCGTGGACAAGAGCAGGTGGC | |
| | | AGCAGGGGAACGTCTTCTCATGCTCCGTGATGCATGAGG | |
| | | CTCTGCACAACCACTACACGCAGAAGAGCCTCTCCCTGTC | |
| | | TCCGGGTAAATGA | |
| 5-B07 λ | 5-B07 | CAGTCTGTGCTGACTCAGCCACCCTCAGCGTCTGGGACCC | 48 |
| light | VL | CCGGGCAGAGGGTCACCATCTCCTGCACTGGGAGCAGCT | |
| chain | | CCAACATCGGGGCAGGTTATGATGTACACTGGTATCAGCA | |
| | | GCTCCCAGGAACGGCCCCCAAACTCCTCATCTATAGGAAT | |
| | | AATCAGCGGCCCTCAGGGGTCCCTGACCGATTCTCTGGCT | |
| | | CCAAGTCTGGCACCTCAGCCTCCCTGGCCATCAGTGGGCT | |
| | | CCGGTCCGAGGATGAGGCTGATTATTACTGTGCAGCATG | |
| | | GGATGACAGCCTGAATGGTTGGGTGTTCGGCGGAGGAAC | |
| | | CAAGCTGACGGTCCTAGGTCAGCCCAAGGCTGCCCCCTC | |
| | | GGTCACTCTGTTCCCGCCCTCCTCTGAGGAGCTTCAAGCC | |
| | | AACAAGGCCACACTGGTGTGTCTCATAAGTGACTTCTACC | |
| | | CGGGAGCCGTGACAGTGGCCTGGAAGGCAGATAGCAGCC | |
| | | CCGTCAAGGCGGGAGTGGAGACCACCACACCCTCCAAAC | |
| | | AAAGCAACAACAAGTACGCGGCCAGCAGCTATCTGAGCCT | |
| | | GACGCCTGAGCAGTGGAAGTCCCACAGAAGCTACAGCTG | |
| | | CCAGGTCACGCATGAAGGGAGCACCGTGGAGAAGACAGT | |
| | | GGCCCCTACAGAATGTTCATGA | |
| 2-C06 | 2-C06 | GAGGTGCAGCTGTTGGAGTCTGGGGGAGGCTTGGTACAG | 49 |
| γ1 | VH | CCTGGGGGGTCCCTGAGACTCTCCTGTGCAGCCTCTGGA | |
| heavy | | TTCACCTTTAGCAGCTATGCCATGAGCTGGGTCCGCCAGG | |
| chain | | CTCCAGGGAAGGGGCTGGAGTGGGTCTCAGGTATTAGTG | |
| | | GCAGCGGTGGGTACATACACTATGCAGACTCCGTGAAGG | |
| | | GCCGGTTCACCATCTCCAGAGACAATTCCAAGAACACGCT | |
| | | GTATCTGCAAATGAACAGCCTGAGAGCCGAGGACACTGC | |
| | | CGTGTATTACTGTGCGACCTATAGCAGTGGCCTGCATGAT | |
| | | GCTTTTGATATCTGGGGCCAGGGTACACTGGTCACCGTGA | |
| | | GCTCAGCCTCCACCAAGGGCCCATCGGTCTTCCCCCTGGC | |
| | | ACCCTCCTCCAAGAGCACCTCTGGGGGCACAGCGGCCCT | |
| | | GGGCTGCCTGGTCAAGGACTACTTCCCCGAACCGGTGAC | |
| | | GGTGTCGTGGAACTCAGGCGCCCTGACCAGCGGCGTGCA | |
| | | CACCTTCCCGGCTGTCCTACAGTCCTCAGGACTCTACTCC | |
| | | CTCAGCAGCGTGGTGACCGTGCCCTCCAGCAGCTTGGGC | |
| | | ACCCAGACCTACATCTGCAACGTGAATCACAAGCCCAGCA | |
| | | ACACCAAGGTGGACAAGAAAGTTGAGCCCAAATCTTGTGA | |
| | | CAAAACTCACACATGCCCACCGTGCCCAGCACCTGAACTC | |
| | | CTGGGGGGACCGTCAGTCTTCCTCTTCCCCCCAAAACCCA | |
| | | AGGACACCCTCATGATCTCCCGGACCCCTGAGGTCACATG | |
| | | CGTGGTGGTGGACGTGAGCCACGAAGACCCTGAGGTCAA | |
| | | GTTCAACTGGTACGTGGACGGCGTGGAGGTGCATAATGC | |
| | | CAAGACAAAGCCGCGGGAGGAGCAGTACAACAGCACGTA | |
| | | CCGTGTGGTCAGCGTCCTCACCGTCCTGCACCAGGACTG | |
| | | GCTGAATGGCAAGGAGTACAAGTGCAAGGTCTCCAACAA | |
| | | AGCCCTCCCAGCCCCCATCGAGAAAACCATCTCCAAAGCC | |
| | | AAAGGGCAGCCCCGAGAACCACAGGTGTACACCCTGCCC | |
| | | CCATCCCGGGATGAGCTGACCAAGAACCAGGTCAGCCTG | |
| | | ACCTGCCTGGTCAAAGGCTTCTATCCCAGCGACATCGCCG | |
| | | TGGAGTGGGAGAGCAATGGGCAGCCGGAGAACAACTACA | |
| | | AGACCACGCCTCCCGTGCTGGACTCCGACGGCTCCTTCTT | |
| | | CCTCTACAGCAAGCTCACCGTGGACAAGAGCAGGTGGCA | |
| | | GCAGGGGAACGTCTTCTCATGCTCCGTGATGCATGAGGCT | |
| | | CTGCACAACCACTACACGCAGAAGAGCCTCTCCCTGTCTC | |
| | | CGGGTAAATGA | |
| 2-C06 λ | 2-C06 | CAGTCTGTGCTGACTCAGCCACCCTCAGCGTCTGGGACCC | 50 |
| light | VL | CCGGGCAGAGGGTCACCATCTCCTGCACTGGGAGCAGCT | |
| chain | | CCAACATCGGGGCAGGTTATGATGTACACTGGTATCAGCA | |
| | | GCTCCCAGGAACGGCCCCCAAACTCCTCATCTATGACAAT | |
| | | AATAAGCGACCCTCAGGGGTCCCTGACCGATTCTCTGGCT | |
| | | CCAAGTCTGGCACCTCAGCCTCCCTGGCCATCAGTGGGCT | |
| | | CCGGTCCGAGGATGAGGCTGATTATTACTGTGCAGCATG | |
| | | GGATGACAGCCTGAATGGTTGGGTGTTCGGCGGAGGAAC | |
| | | CAAGCTGACGGTCCTAGGTCAGCCCAAGGCTGCCCCCTC | |
| | | GGTCACTCTGTTCCCGCCCTCCTCTGAGGAGCTTCAAGCC | |
| | | AACAAGGCCACACTGGTGTGTCTCATAAGTGACTTCTACC | |
| | | CGGGAGCCGTGACAGTGGCCTGGAAGGCAGATAGCAGCC | |
| | | CCGTCAAGGCGGGAGTGGAGACCACCACACCCTCCAAAC | |
| | | AAAGCAACAACAAGTACGCGGCCAGCAGCTATCTGAGCCT | |
| | | GACGCCTGAGCAGTGGAAGTCCCACAGAAGCTACAGCTG | |
| | | CCAGGTCACGCATGAAGGGAGCACCGTGGAGAAGACAGT | |
| | | GGCCCCTACAGAATGTTCATGA | |
| 2-F09 | 2-F09 | GAGGTGCAGCTGTTGGAGTCTGGGGGAGGCTTGGTACAG | 51 |
| γ1 | VH | CCTGGGGGGTCCCTGAGACTCTCCTGTGCAGCCTCTGGA | |
| heavy | | TTCACCTTCAAAGCCTATAGCATGAGCTGGATCCGCCAGG | |
| chain | | CTCCAGGGAAGGGGCTGGAGTGGGTCTCAGGTATCAGTA | |
| | | ACACGGGAGGTAGCACAGACTTCGCAGACTCCGTGAAGG | |
| | | GCCGGTTCACCATCTCCAGAGACAATTCCAAGAACACGCT | |
| | | GTATCTGCAAATGAACAGCCTGAGAGCCGAGGACACTGC | |
| | | CATGTATTACTGTGCGAGATTGGGATATAGTGGCTACGAC | |
| | | GACCGTGGTATGGACGTCTGGGGCCAAGGTACACTGGTC | |
| | | ACCGTGAGCTCAGCCTCCACCAAGGGCCCATCGGTCTTCC | |
| | | CCCTGGCACCCTCCTCCAAGAGCACCTCTGGGGGCACAG | |
| | | CGGCCCTGGGCTGCCTGGTCAAGGACTACTTCCCCGAAC | |
| | | CGGTGACGGTGTCGTGGAACTCAGGCGCCCTGACCAGCG | |
| | | GCGTGCACACCTTCCCGGCTGTCCTACAGTCCTCAGGACT | |
| | | CTACTCCCTCAGCAGCGTGGTGACCGTGCCCTCCAGCAG | |
| | | CTTGGGCACCCAGACCTACATCTGCAACGTGAATCACAAG | |
| | | CCCAGCAACACCAAGGTGGACAAGAAAGTTGAGCCCAAA | |
| | | TCTTGTGACAAAACTCACACATGCCCACCGTGCCCAGCAC | |
| | | CTGAACTCCTGGGGGGACCGTCAGTCTTCCTCTTCCCCCC | |
| | | AAAACCCAAGGACACCCTCATGATCTCCCGGACCCCTGAG | |
| | | GTCACATGCGTGGTGGTGGACGTGAGCCACGAAGACCCT | |
| | | GAGGTCAAGTTCAACTGGTACGTGGACGGCGTGGAGGTG | |
| | | CATAATGCCAAGACAAAGCCGCGGGAGGAGCAGTACAAC | |
| | | AGCACGTACCGTGTGGTCAGCGTCCTCACCGTCCTGCACC | |
| | | AGGACTGGCTGAATGGCAAGGAGTACAAGTGCAAGGTCT | |
| | | CCAACAAAGCCCTCCCAGCCCCCATCGAGAAAACCATCTC | |
| | | CAAAGCCAAAGGGCAGCCCCGAGAACCACAGGTGTACAC | |
| | | CCTGCCCCCATCCCGGGATGAGCTGACCAAGAACCAGGT | |
| | | CAGCCTGACCTGCCTGGTCAAAGGCTTCTATCCCAGCGAC | |
| | | ATCGCCGTGGAGTGGGAGAGCAATGGGCAGCCGGAGAA | |
| | | CAACTACAAGACCACGCCTCCCGTGCTGGACTCCGACGG | |
| | | CTCCTTCTTCCTCTACAGCAAGCTCACCGTGGACAAGAGC | |
| | | AGGTGGCAGCAGGGGAACGTCTTCTCATGCTCCGTGATG | |
| | | CATGAGGCTCTGCACAACCACTACACGCAGAAGAGCCTCT | |
| | | CCCTGTCTCCGGGTAAATGA | |
| 2-F09 λ | 2-F09 | CAGTCTGTGCTGACTCAGCCACCCTCAGCGTCTGGGACCC | 52 |
| light | VL | CCGGGCAGAGGGTCACCATCTCTTGTTCTGGAAGCAGCTC | |
| chain | | CAACATCGGAAGTAATTATGTGTACTGGTATCAGCAGCTC | |
| | | CCAGGAACGGCCCCCAAACTCCTCATCTATGGTAACAGCA | |
| | | ATCGGCCCTCAGGGGTCCCTGACCGATTCTCTGGCTCCAA | |
| | | GTCTGGCACCTCAGCCTCCCTGGCCATCAGTGGGCTCCG | |
| | | GTCCGAGGATGAGGCTGATTATTACTGCCAGTCCTATGAC | |
| | | AGCAGCCTGAGTGGTCCTGTGGTATTCGGCGGAGGAACC | |
| | | AAGCTGACGGTCCTAGGTCAGCCCAAGGCTGCCCCCTCG | |
| | | GTCACTCTGTTCCCGCCCTCCTCTGAGGAGCTTCAAGCCA | |
| | | ACAAGGCCACACTGGTGTGTCTCATAAGTGACTTCTACCC | |
| | | GGGAGCCGTGACAGTGGCCTGGAAGGCAGATAGCAGCCC | |
| | | CGTCAAGGCGGGAGTGGAGACCACCACACCCTCCAAACA | |
| | | AAGCAACAACAAGTACGCGGCCAGCAGCTATCTGAGCCT | |
| | | GACGCCTGAGCAGTGGAAGTCCCACAGAAGCTACAGCTG | |
| | | CCAGGTCACGCATGAAGGGAGCACCGTGGAGAAGACAGT | |
| | | GGCCCCTACAGAATGTTCATGA | |

In some embodiments, it is advantageous that the antibody molecule binds both to human CTLA-4 (hCTLA-4) and to cynomolgus monkey CTLA-4 (cmCTLA-4 or cyno CTLA-4). Cross-reactivity with CTLA-4 expressed on cells in cynomolgus monkey, also called crab-eating macaque or Macaca fascicularis, may be advantageous since this enables testing of the antibody molecule in monkey without having to use a surrogate antibody, which particular focus on tolerability.

In some embodiments, it is advantageous that the antibody molecule binds both to human CTLA-4 (hCTLA-4) and to murine CTLA-4 (mCTLA-4). This may be advantageous since this enables testing of the antibody molecule in mice, with particular focus on effect and pharmacodynamics, without having to use a surrogate antibody.

In some embodiments, the antibody molecule binds to all three hCTLA-4, cmCTLA-4 and mCTLA-4.

In some embodiments, it is necessary to use a surrogate antibody to test an antibody molecule's functional activity in relevant in vivo models in mice. To ensure the comparability between the antibody molecule's effect in humans and the in vivo results for the surrogate antibody in mice, it is essential to select a functionally equivalent surrogate antibody having the same in vitro characteristics as the human antibody molecule.

In some embodiments, the first antibody molecule that specifically binds to CTLA-4 does not bind human CD28.

As discussed herein, the second antibody may specifically bind to PD-1. In some embodiments, the antibody molecule that specifically binds to PD-1 is selected from one or more of the following, non-limiting examples of anti-PD-1 antibodies:
Pembrolizumab (currently approved for use);
Nivolumab (currently approved for use);
Cemiplimab (currently approved for use);
Camrelizumab (currently approved for use);
Spartalizumab (currently in clinical development);
Dostarlimab (currently in clinical development);
Tislelizumab (currently in clinical development);
JTX-4014 (currently in clinical development);
Sintilimab (IBI308) (currently in clinical development);
Toripalimab (JS 001) (currently in clinical development);
AMP-224 (currently in clinical development);
AMP-514 (MEDI0680) (currently in clinical development).

In a preferred embodiment, the antibody that binds specifically to PD-1 is Pembrolizumab, Nivolumab, Cemiplimab, or Camrelizumab. In some embodiments, the antibody that binds specifically to PD-1 is a combination of two or more of these antibodies. In a preferred embodiment, the antibody that binds specifically to PD-1 is Pembrolizumab.

In some embodiments, the anti-PD-1 antibody molecule is a human antibody molecule. In some embodiments, the anti-PD-1 antibody molecule is a humanized antibody molecule.

In some embodiments, the anti-PD-1 antibody molecule is an antibody molecule of human origin, meaning that it originates from a human antibody molecule which then has been modified.

In some embodiments, the anti-PD-1 antibody molecule is a human IgG1 antibody.

In some embodiments, the anti-PD-1 antibody is an antibody in the form of a human IgG1 antibody showing improved binding to one or several activatory Fc receptors and/or being engineered for improved binding to one or several activatory Fc receptors; accordingly, in some embodiments, the anti-PD-1 antibody is an Fc-engineered human IgG1 antibody.

In some embodiments, the anti-PD-1 antibody is a murine or a humanized murine IgG2a antibody.

In some embodiments, the second antibody molecule that specifically binds to PD-1 is selected from the group consisting of a human antibody molecule, a humanized antibody molecule, and an antibody molecule of human origin.

In some embodiments, the second antibody molecule that specifically binds to PD-1 is selected from the group consisting of a full-size antibody, a chimeric antibody, a single chain antibody, and an antigen-binding fragment thereof (e.g. a Fab, a Fv, an scFv, a Fab′, and a (Fab′)₂).

In some embodiments, the second antibody molecule that specifically binds to PD-1 is selected from the group consisting of a human IgG antibody, a humanized IgG antibody and an IgG antibody of human origin.

In some embodiments, the anti-PD-1 antibody is a murine antibody that is cross-reactive with human PD-1.

In some embodiments, the anti-PD-1 antibody is a monoclonal antibody or an antibody molecule of monoclonal origin. In some embodiments, the anti-PD-1 antibody is a polyclonal antibody.

In some embodiments, the second antibody molecule may specifically bind to PD-L1. In some embodiments, the antibody molecule that specifically binds to PD-L1 is selected from one or more of the following, non-limiting examples of anti-PD-L1 antibodies:
Atezolizumab (currently approved for use);
Durvalumab (currently approved for use);
Avelumab (currently approved for use);
CS1001 (currently in clinical development);
KN035 (Envafolimab)—a PD-L1 antibody with subcutaneous formulation currently under clinical evaluations in the US, China, and Japan;
CK-301 (currently in clinical development by Checkpoint Therapeutics)

In a preferred embodiment, the antibody that binds specifically to PD-L1 is Atezolizumab, Durvalumab, or Avelumab. In some embodiments, the antibody that binds specifically to PD-L1 is a combination of two or more of these antibodies.

In some embodiments, the anti-PD-L1 antibody molecule is a human antibody molecule.

In some embodiments, the anti-PD-L1 antibody molecule is a humanized antibody molecule.

In some embodiments, the anti-PD-L1 antibody molecule is an antibody molecule of human origin, meaning that it originates from a human antibody molecule which then has been modified.

In some embodiments, the anti-PD-L1 antibody molecule is a human IgG1 antibody.

In some embodiments, the anti-PD-L1 antibody is an antibody in the form of a human IgG1 antibody showing improved binding to one or several activatory Fc receptors and/or being engineered for improved binding to one or several activatory Fc receptors; accordingly, in some embodiments, the anti-PD-L1 antibody is an Fc-engineered human IgG1 antibody.

In some embodiments, the anti-PD-L1 antibody is a murine or a humanized murine IgG2a antibody.

In some embodiments, the second antibody molecule that specifically binds to PD-L1 is selected from the group consisting of a human antibody molecule, a humanized antibody molecule, and an antibody molecule of human origin.

In some embodiments, the second antibody molecule that specifically binds to PD-L1 is selected from the group consisting of a full-size antibody, a chimeric antibody, a single chain antibody, and an antigen-binding fragment thereof (e.g. a Fab, a Fv, an scFv, a Fab′, and a (Fab′)₂).

In some embodiments, the second antibody molecule that specifically binds to PD-L1 is selected from the group consisting of a human IgG antibody, a humanized IgG antibody and an IgG antibody of human origin.

In some embodiments, the anti-PD-L1 antibody is a murine antibody that is cross-reactive with human PD-L1.

In some embodiments, the anti-PD-L1 antibody is a monoclonal antibody or an antibody molecule of monoclonal origin. In some embodiments, the anti-PD-L1 antibody is a polyclonal antibody.

As described herein, the invention involves an oncolytic virus capable of expressing a first antibody molecule that specifically binds to CTLA-4.

As used herein, the term “oncolytic” refers to the capacity of a virus of selectively replicating in dividing cells (e.g. a proliferative cell such as a cancer cell) with the aim of slowing the growth and/or lysing said dividing cell, either in vitro or in vivo, while showing no or minimal replication in non-dividing (e.g. normal or healthy) cells.

“Replication” (or any form of replication such as “replicate” and “replicating”, etc.) means duplication of a virus that can occur at the level of nucleic acid or, preferably, at the level of infectious viral particle. Such an oncolytic virus can be obtained from any member of virus identified at present time. It may be a native virus that is naturally oncolytic or may be engineered by modifying one or more viral genes so-as to increase tumor selectivity and/or preferential replication in dividing cells, such as those involved in DNA replication, nucleic acid metabolism, host tropism, surface attachment, virulence, lysis and spread (see for example Wong et al., 2010, Viruses 2:78-106). One may also envisage placing one or more viral gene(s) under the control of event or tissue-specific regulatory elements (e.g. promoter).

Exemplary oncolytic viruses include without limitation: reovirus; Seneca Valley virus (SVV); vesicular stomatitis virus (VSV); Newcastle disease virus (NDV); herpes simplex virus (HSV); morbillivirus; adenovirus; poxvirus; retrovirus; measles virus; foamy virus; alpha virus; lentivirus; influenza virus; Sinbis virus; myxoma virus; rhabdovirus; picornavirus; coxsackievirus; parvovirus or the like. Such viruses are known to those skilled in the arts of medicine and virology.

In some embodiments, such an oncolytic virus is obtained from a herpes virus. The Herpesviridae are a large family of DNA viruses that all share a common structure and are composed of relatively large double-stranded, linear DNA genomes encoding 100-200 genes encapsidated within an icosahedral capsid which is enveloped in a lipid bilayer membrane. Although the oncolytic herpes virus can be derived from different types of HSV, particularly preferred are HSV1 and HSV2. The herpes virus may be genetically modified so-as to restrict viral replication in tumors or reduce its cytotoxicity in non-dividing cells. For example, any viral gene involved in nucleic acid metabolism may be inactivated, such as thymidine kinase (Martuza et al., 1991, Science 252:854-6), ribonucleotide reductase (RR) (Mineta et al., 1994, Cancer Res. 54:3363-66), or uracil-N-glycosylase (Pyles et al., 1994, J. Virol. 68:4963-72). Another aspect involves viral mutants with defects in the function of genes encoding virulence factors such as the ICP34.5 gene (Chambers et al., 1995, Proc. Natl. Acad. Sci. USA 92:1411-5). Representative examples of oncolytic herpes virus include NV1020 (e.g. Geevarghese et al., 2010, Hum. Gene Ther. 21 (9): 1119-28) and T-VEC (Harrington et al., 2015, Expert Rev. Anticancer Ther. 15 (12): 1389-1403).

In some embodiments, such an oncolytic virus is obtained from an adenovirus. Methods are available in the art to engineer oncolytic adenoviruses. An advantageous strategy includes the replacement of viral promoters with tumor-selective promoters or modifications of the E1 adenoviral gene product(s) to inactivate its/their binding function with p53 or retinoblastoma (Rb) protein that are altered in tumor cells. In the natural context, the adenovirus E1B55 kDa gene cooperates with another adenoviral product to inactivate p53 (p53 is frequently dysregulated in cancer cells), thus preventing apoptosis. Representative examples of oncolytic adenoviruses include ONYX-015 (e.g. Khuri et al., 2000, Nat. Med 6 (8): 879-85) and H101 also named Oncorine (Xia et al., 2004, Ai Zheng 23 (12): 1666-70).

In some embodiments, such an oncolytic virus is an oncolytic poxvirus. As used herein the term “poxvirus” refers to a virus belonging to the Poxviridae family, with a specific preference for a poxvirus belonging to the Chordopoxviridae subfamily and more preferably to the Orthopoxvirus genus. Vaccinia virus, cowpox virus, canarypox virus, ectromelia virus, myxoma virus are particularly appropriate in the context of the invention. Genomic sequences of such poxviruses are available in the art and specialized databases (e.g. Genbank under accession number NC_006998, NC_003663 or AF482758.2, NC_005309, NC_004105, NC_001132 respectively).

In specific and preferred embodiments, such an oncolytic poxvirus is an oncolytic vaccinia virus. Vaccinia viruses are members of the poxvirus family characterized by a 200 kb double-stranded DNA genome that encodes numerous viral enzymes and factors that enable the virus to replicate independently from the host cell machinery. The majority of vaccinia virus particles is intracellular (IMV for intracellular mature virion) with a single lipid envelop and remains in the cytosol of infected cells until lysis. The other infectious form is a double enveloped particle (EEV for extracellular enveloped virion) that buds out from the infected cell without lysing it. Although it can derive from any vaccinia virus strain, Elstree, Wyeth, Copenhagen, Lister and Western Reserve strains are particularly preferred. The gene nomenclature used herein is that of Copenhagen vaccinia strain unless otherwise indicated. However, correspondence between Copenhagen and other vaccinia strains are generally available in the literature.

Preferably, such an oncolytic vaccinia virus is modified by altering one or more viral gene(s). Said modification(s) preferably lead(s) to the absence of synthesis or the synthesis of a defective viral protein unable to ensure the activity of the protein produced under normal conditions by the unmodified gene. Exemplary modifications are disclosed in the literature with the goal of altering viral genes involved in DNA metabolism, host virulence, IFN pathway (e.g. Guse et al., 2011, Expert Opinion Biol. Ther.11 (5): 595-608) and the like. Modifications for altering a viral locus encompass deletion, mutation and/or substitution of one or more nucleotide(s) (contiguous or not) within the viral gene or its regulatory elements. Modification(s) can be made by a number of ways known to those skilled in the art using conventional recombinant techniques.

More preferably, such an oncolytic vaccinia virus is modified by altering the thymidine kinase-encoding gene (locus J2R). The thymidine kinase (TK) enzyme is involved in the synthesis of deoxyribonucleotides. TK is needed for viral replication in normal cells as these cells have generally low concentration of nucleotides whereas it is dispensable in dividing cells which contain high nucleotide concentration.

Alternatively, or in combination, such an oncolytic vaccinia virus is modified by altering at least one gene or both genes encoding ribonucleotide reductase (RR). In the natural context, this enzyme catalyses the reduction of ribonucleotides to deoxyribonucleotides that represents a crucial step in DNA biosynthesis. The viral enzyme is similar in subunit structure to the mammalian enzyme, being composed of two heterologous subunits, designed R1 and R2 encoded respectively by the I4L and F4L locus. In the context of the invention, either the I4L gene (encoding the R1 large subunit) or the F4L gene (encoding the R2 small subunit) or both may be inactivated (e.g. as described in WO2009/065546 and Foloppe et al., 2008, Gene Ther., 15:1361-71). Sequences for the J2R, I4L and F4L genes and their locations in the genome of various poxviruses are available in public databases.

Therefore, in some embodiments, the oncolytic virus is defective for thymidine kinase (TK) and/or ribonucleotide reductase (RR) activity. In some embodiments, the oncolytic virus is a vaccinia virus defective for thymidine kinase (TK) and/or ribonucleotide reductase (RR) activity.

In some embodiments, the oncolytic virus comprises a nucleotide sequence encoding the first antibody molecule as defined herein.

In some embodiments, such an oncolytic virus comprises nucleotide sequences encoding amino acid sequence having at least 80% identity with a sequence set out in Table 2 above. In some embodiments, such an oncolytic virus comprises an amino acid sequence having at least 85% identity with a sequence set out in Table 2 above. In some embodiments, such an oncolytic virus comprises an amino acid sequence having at least 90% identity with a sequence set out in Table 2 above. In some embodiments, such an oncolytic virus comprises an amino acid sequence having at least 95% identity with a sequence set out in Table 2 above.

In some embodiments, such an oncolytic virus comprises nucleotide sequences encoding SEQ. ID. NO: 20 and ID. NO: 21. In some embodiments, such an oncolytic virus comprises nucleotide sequences encoding SEQ. ID. NO: 27 and ID. NO: 28. In some embodiments, such a oncolytic virus comprises nucleotide sequences encoding SEQ. ID. NO: 33 and ID. NO: 34. In some embodiments, such a oncolytic virus comprises nucleotide sequences encoding SEQ. ID. NO: 41 and ID. NO: 42.

In some embodiments, such an oncolytic virus comprises nucleotide sequences having at least 80% identity with a sequence set out in Table 4 above. In some embodiments, such an oncolytic virus comprises nucleotide sequences having at least 85% identity with a sequence set out in table 4 above. In some embodiments, such an oncolytic virus comprises nucleotide sequences having at least 90% identity with a sequence set out in table 4 above. In some embodiments, such an oncolytic virus comprises nucleotide sequences having at least 95% identity with a sequence set out in table 4 above.

In some embodiments, the nucleotide sequence comprises or consists of a sequence selected from the group consisting of SEQ ID NOs: 45-52. In some embodiments, such a oncolytic virus comprises SEQ. ID. NO: 45 and 46. In some embodiments, such a oncolytic virus comprises SEQ. ID. NO: 47 and 48. In some embodiments, such a oncolytic virus comprises SEQ. ID. NO: 49 and 50. In some embodiments, such a oncolytic virus comprises SEQ. ID. NO: 51 and 52.

Some oncolytic viruses have capacity to host large enough DNA insertions to accommodate integration of full-length human antibody sequences. Attenuated Vaccinia viruses and Herpes Simplex Viruses are examples of therapeutic oncolytic viruses whose genome is sufficiently large to permit integration of full-length IgG antibody sequences (Chan and McFadden 2014, Bommareddy, Shettigar et al. 2018). Full-length IgG antibodies have successfully been integrated into oncolytic Vaccinia virus, resulting in expression and extracellular release (production) of full-length IgG antibodies upon infection of virus-susceptible host cells e.g. cancer cells (Kleinpeter, et al. 2016). Adenoviruses can also be engineered to encode full-length IgG antibodies that are functionally produced and secreted upon cellular infection (Marino et al. 2017).

In a preferred embodiment, such an oncolytic virus is a poxvirus (e.g. a vaccinia virus) defective for TK activity (resulting from alteration of the J2R locus) or defective for both TK and RR activities (resulting from alteration of both the J2R locus and at least one of the RR-encoding I4L and/or F4L locus) and comprising (a) nucleotide sequences encoding SEQ. ID. NO: 20 and ID. NO: 21 or (b) nucleotide sequences encoding SEQ. ID. NO: 27 and ID. NO: 28 or (c) nucleotide sequences encoding SEQ. ID. NO: 33 and ID. NO: 34 or (d) nucleotide sequences encoding SEQ. ID. NO: 41 and ID. NO: 42.

In some embodiments, the TK and RR activities may be disrupted by the introduction of nucleotide sequences encoding the first antibody molecule within the relevant loci (i.e. the J2R, I4L and/or F4L loci. In some preferred embodiments, the virus comprises a nucleotide sequence encoding the heavy chain of the first antibody molecule inserted at the viral J2R locus and/or comprises a nucleotide sequence encoding the light chain of the first antibody molecule inserted at the viral I4L locus.

When appropriate, it may be advantageous that the nucleotide sequence(s) inserted in the oncolytic virus described herein include(s) additional regulatory elements to facilitate expression, trafficking and biological activity. For example, a signal peptide may be included for facilitating secretion outside the producer cell (e.g. infected cell). The signal peptide is typically inserted at the N-terminus of the encoded polypeptide immediately after the Met initiator. The choice of signal peptides is wide and is accessible to persons skilled in the art. For example, signal peptides originating from another immunoglobin (e.g. a heavy chain IgG) can be used in the context of the invention to allow secretion of the anti-CTLA4 antibody described herein outside the producer cell. For illustrative purposes, one may refer to SEQ ID NO: 53 and SEQ ID NO: 54 comprising the light chain and the heavy chain of the 4-E03 antibody described herein equipped with IgG-originating peptide signals.

A particularly preferred oncolytic virus is a vaccinia virus (e.g. Copenhagen strain) defective for both TK and RR activities (e.g. resulting from alteration of both the J2R locus and the I4L loci) and comprising nucleotide sequences encoding SEQ. ID. NO: 20 and SEQ ID. NO: 21 or SEQ. ID. NO: 53 and SEQ ID. NO: 54.

In some embodiments, such an oncolytic virus may further comprise additional nucleotide sequence(s) of therapeutic interest such as nucleotide sequence(s) encoding immunomodulatory polypeptide(s) (i.e. a polypeptide involved in stimulating an immune response either directly or indirectly). Representative examples of suitable immunomodulatory polypeptides include, without any limitation, cytokines and chemokines with a specific preference for granulocyte macrophage colony stimulating factor (GM-CSF) and particularly human, non-human primate or murine GM-CSF.

Therefore, in some embodiments, the oncolytic virus (eg. a vaccinia virus) capable of expressing a first antibody molecule that specifically binds to CTLA-4 further comprises a nucleotide sequence encoding a GM-CSF, preferably human GM-CSF (e.g. having SEQ ID NO: 55 or SEQ ID NO: 56) or a murine GM-CSF (e.g. having SEQ ID NO: 57 or SEQ ID NO: 58).

The additional nucleotide sequence may be easily obtained by standard molecular biology techniques (e.g. PCR amplification, cDNA cloning, chemical synthesis) using sequence data accessible in the art and the information provided herein. A particularly preferred oncolytic virus is a vaccinia virus (e.g. Copenhagen strain) defective for both TK and RR activities (resulting from alteration of both the J2R locus and the I4L loci) and comprising nucleotide sequences encoding SEQ. ID. NO: 20 and ID. NO: 21 or SEQ. ID. NO: 53 and SEQ ID. NO: 54 and a nucleotide sequence encoding a GM-CSF, with a specific preference for a human GM-CSF (e.g. having SEQ ID NO: 55 or SEQ ID NO: 56) or a murine GM-CSF (e.g. having SEQ ID NO: 57 or SEQ ID NO: 58).

The following table provides sequences of GM-CSF referred to herein:

| | | SEQ. |
|---|---|---|
| | | ID. |
| | Sequence | NO |
| Human GM- | APARSPSPSTQPWEHVNAIQEARRLLNLSRDTAAEMNETVEVISEM | 55 |
| CSF without | FDLQEPTCLQTRLELYKQGLRGSLTKLKGPLTMMASHYKQHCPPTPE | |
| signal | TSCATQTITFESFKENLKDFLLVIPFDCWEPVQE | |
| peptide | | |
| Human GM- | MWLQSLLLLGTVACSISAPARSPSPSTQPWEHVNAIQEARRLLNLS | 56 |
| CSF with | RDTAAEMNETVEVISEMFDLQEPTCLQTRLELYKQGLRGSLTKLKGP | |
| signal | LTMMASHYKQHCPPTPETSCATQTITFESFKENLKDFLLVIPFDCWE | |
| peptide | PVQE | |
| Murine GM- | APTRSPITVTRPWKHVEAIKEALNLLDDMPVTLNEEVEVVSNEFSFK | 57 |
| CSF without | KLTCVQTRLKIFEQGLRGNFTKLKGALNMTASYYQTYCPPTPETDCE | |
| signal | TQVTTYADFIDSLKTFLTDIPFECKKPVQK | |
| peptide | | |
| Murine GM- | MWLQNLLFLGIVVYSLSAPTRSPITVTRPWKHVEAIKEALNLLDDMP | 58 |
| CSF with | VTLNEEVEVVSNEFSFKKLTCVQTRLKIFEQGLRGNFTKLKGALNMT | |
| signal | ASYYQTYCPPTPETDCETQVTTYADFIDSLKTFLTDIPFECKKPVQK | |
| peptide | | |

In addition, the nucleotide sequences to be inserted in such an oncolytic virus can be optimized for providing high level expression in a particular host cell or subject by modifying one or more codon(s). Further to optimization of the codon usage, various modifications may also be envisaged so as to prevent clustering of rare, non-optimal codons being present in concentrated areas and/or to suppress or modify “negative” sequence elements which are expected to negatively influence expression levels. Such negative sequence elements include without limitation the regions having very high (>80%) or very low (<30%) GC content; AT-rich or GC-rich sequence stretches; unstable direct or inverted repeat sequences; R A secondary structures; and/or internal cryptic regulatory elements such as internal TATA-boxes, chi-sites, ribosome entry sites, and/or splicing donor/acceptor sites.

In some embodiments, the nucleotide sequence(s) are placed under the control of suitable regulatory elements for their proper expression in a host cell or subject. As used herein, the term “regulatory elements” refers to any element that allows, contributes or modulates the expression of the encoding nucleotide sequence(s) in a given host cell or subject, including their replication, duplication, transcription, splicing, translation, stability and/or transport in or outside the expressing cell. It will be appreciated by those skilled in the art that the choice of the regulatory elements can depend on such factors as the nucleotide sequence itself, the virus into which it is inserted, the host cell or subject, the level of expression desired, etc. The promoter is of special importance. In the context of the invention, it can be constitutive directing expression of the nucleotide sequence that it controls in many types of host cells or specific to certain host cells or regulated in response to specific events or exogenous factors (e.g. by temperature, nutrient additive, hormone, etc.) or according to the phase of a viral cycle (e.g. late or early). Promoters adapted to virus-mediated expression are known in the art.

Representative examples for expression by an oncolytic poxvirus include without limitation the vaccinia p7.5K, pH5.R, p11K7.5, TK, p28, p11, pB2R, pA35R, K1L and pSE/L promoters (Erbs et al., 2008, Cancer Gene Ther. 15 (1): 18-28; Orubu et al. 2012, PloS One 7: e40167), early/late chimeric promoters and synthetic promoters (Chakrabarti et al., 1997, Biotechniques 23:1094-7; Hammond et al, 1997, J. Virol Methods 66:135-8; and Kumar and Boyle, 1990, Virology 179:151-8). In preferred embodiments, the nucleotide sequences of the light and heavy chains of the antibody described herein are respectively placed under the control of promoters having the same transcriptional strength, and preferably under the control of the same promoter (e.g. p7.5K such as the one described in SEQ ID NO: 59 or pH5.R such as the one described in SEQ ID NO: 60) to obtain a similar level of expression for both chains and therefore an optimal assembly of the antibody as a hetero-tetrameric protein (i.e. to avoid excess of non-associated chain). The additional nucleotide sequence (e.g. encoding GM-CSF) can be placed under a different promoter (e.g. pSE/L such as the one described in SEQ ID NO: 61).

The following table provides sequences of the promoters referred to above:

| | | SEQ |
|---|---|---|
| | | ID |
| | Sequence | NO. |
| Promoter | CCACCCACTTTTTATAGTAAGTTTTTCACCCATAAATAATAAATAC | 59 |
| p7.5K | AATAATTAATTTCTCGTAAAAGTAGAAAATATATTCTAATTTATTGC | |
| | ACGGTAAGGAAGTAGAATCATAAAGAACAGT | |
| Promoter | TTTATTCTATACTTAAAAAATGAAAATAAATACAAAGGTTCTTGAG | 60 |
| PH5.R | GGTTGTGTTAAATTGAAAGCGAGAAATAATCATAAATTATTTCATT | |
| | ATCGCGATATCCGTTAAGTTTG | |
| Promoter | AAAAATTGAAATTTTATTTTTTTTTTTTGGAATATAAATA | 61 |
| pSE/L | | |

Insertion of the nucleotide sequence(s) (possibly equipped with appropriate regulatory elements) in the genome of such an oncolytic virus is made by conventional means, either using appropriate restriction enzymes or, preferably by homologous recombination. The nucleotide sequence(s) can independently be inserted at any location of the viral genome. Various sites of insertion may be considered, e.g. in a non-essential viral gene, in an intergenic region, or in a non-coding portion of the genome of such an oncolytic virus. J2R locus and/or I4L locus is particularly appropriate for an oncolytic virus being a poxvirus (e.g. a oncolytic vaccinia virus). Upon insertion of the nucleotide sequence(s) into the viral genome, the viral locus at the insertion site may be deleted at least partially. In one embodiment, this deletion or partial deletion may result in suppressed expression of the viral gene product encoded by the entirely or partially deleted locus resulting in a defective virus for said virus function. A particularly preferred oncolytic virus is a TK and/or RR defective vaccinia virus comprising the cassette encoding the heavy chain inserted at the J2R locus and the cassette encoding the light chain inserted at the I4L locus. The cassette encoding the additional GM-CSF-encoding nucleotide sequence can be inserted in another location of the virus genome or in J2R or I4L locus, with a preference for insertion at the I4L locus.

The present invention also provides a method for generating such an oncolytic virus described herein, and particularly an oncolytic poxvirus, in a suitable host cell (producer cell). In some embodiments, such a method comprises one or more step(s) of homologous recombination between a virus genome and a transfer plasmid comprising the nucleotide sequence(s) to be inserted (possibly with regulatory elements) flanked in 5′ and 3′ with viral sequences respectively present upstream and downstream the insertion site. Said transfer plasmid can be generated and introduced into the host cell by routine techniques (e.g. by transfection). The virus genome can be introduced into the host cell by infection. The size of each flanking viral sequence may vary from at least 100 bp and at most 1500 bp on each side of the nucleotide sequence (preferably from 200 to 550 bp and more preferably from 250 to 500 bp). Homologous recombination permitting to generate such an oncolytic virus is preferably carried out in cultured cell lines (e.g. HeLa, Vero) or in chicken embryonic fibroblasts (CEF) cells obtained from embryonated eggs.

In some embodiments, the identification of the oncolytic virus having incorporated the anti-CTLA4 encoding nucleotide sequences and possibly the additional nucleotide sequence (e.g. GM-CSF) may be facilitated by the use of a selection and/or a detectable gene.

In preferred embodiments, the transfer plasmid further comprises a selection marker with a specific preference for the GPT gene (encoding a guanine phosphoribosyl transferase) permitting growth in a selective medium (e.g. in the presence of mycophenolic acid, xanthine and hypoxanthine) or a detectable gene encoding a detectable gene product such as GFP, e-GFP or mCherry. In addition, the use of an endonuclease capable of providing a double-stranded break in said selection or detectable gene may also be considered. Said endonuclease may be in the form of a protein or expressed by an expression vector.

Once generated, such an oncolytic virus can be amplified into a suitable host cell using conventional techniques including culturing the transfected or infected host cell under suitable conditions so as to allow the production and recovery of infectious particles.

The present invention also relates to a method for producing the oncolytic virus described herein. Preferably said method comprises the steps of a) preparing a producer cell line, b) transfecting or infecting the prepared producer cell line with the oncolytic virus, c) culturing the transfected or infected producer cell line under suitable conditions so as to allow the production of the virus, d) recovering the produced virus from the culture of said producer cell line and optionally e) purifying said recovered virus.

In some embodiments, the producer cell is selected from the group consisting of mammalian (e.g. human or non-human) cells such as Hela cells (e.g. ATCC-CRM-CCL-2™ or ATCC-CCL-2.2™), HER96, PER-C6 (Fallaux et al., 1998, Human Gene Ther. 9:1909-17), hamster cell lines such as BHK-21 (ATCC CCL-10) etc. and avian cells such as those described in WO2005/042728, WO2006/108846, WO2008/129058, WO2010/130756, WO2012/001075 as well as a primary chicken embryo fibroblast (CEF) prepared from chicken embryos obtained from fertilized eggs. Producer cells are preferably cultured in an appropriate medium which can, if needed, be supplemented with serum and/or suitable growth factor(s) or not (e.g. a chemically defined medium preferably free from animal- or human-derived products). An appropriate medium may be easily selected by those skilled in the art depending on the producer cells. Such media are commercially available. Producer cells are preferably cultured at a temperature comprised between +30° C. and +38° C. (more preferably at approximately +37° C.) for between 1 and 8 days before infection. If needed, several passages of 1 to 8 days may be made in order to increase the total number of cells.

In step b), producer cells are infected by the oncolytic virus under appropriate conditions using an appropriate multiplicity of infection (MOI) to permit productive infection of producer cells. For illustrative purposes, an appropriate MOI ranges from 10⁻³ to 20, with a specific preference for a MOI comprises from 0.01 to 5 and more preferably 0.03 to 1. Infection step is carried out in a medium which may be the same as or different from the medium used for the culture of producer cells.

In step c), infected producer cells are then cultured under appropriate conditions well known to those skilled in the art until progeny virus particles is produced. Culture of infected producer cells is also preferably performed in a medium which may be the same as or different from the medium/media used for culture of producer cells and/or for infection step, at a temperature between +32° C. and +37° C., for 1 to 5 days.

In step d), the virus particles produced in step c) are collected from the culture supernatant and/or the producer cells. Recovery from producer cells may require a step allowing the disruption of the producer cell membrane to allow the liberation of the virus. The disruption of the producer cell membrane can be induced by various techniques well known to those skilled in the art, including but not limited to freeze/thaw, hypotonic lysis, sonication, microfluidization, high shear (also called high speed) homogenization or high-pressure homogenization.

The recovered oncolytic virus may be at least partially purified before being distributed in doses and used as described herein. A vast number of purification steps and methods is available in the art, including e.g. clarification, enzymatic treatment (e.g. endonuclease, protease, etc.), chromatographic and filtration steps. Appropriate methods are described in the art (see e.g. WO2007/147528; WO2008/138533, WO2009/100521, WO2010/130753, WO2013/022764).

In one embodiment, the present invention also provides a cell infected with the oncolytic virus capable of expressing the first antibody molecule described herein.

The combination of an oncolytic virus capable of expressing a first antibody molecule that specifically binds to CTLA-4, and a second antibody molecule that specifically binds to PD-1 and/or PD-1, is for use in the treatment of cancer in a patient wherein the cancer comprises or consists of a cold tumor.

We include that the subject could be mammalian or non-mammalian. Preferably, the mammalian subject is a human or is a non-mammalian, such as a horse, or a cow, or a sheep, or a pig, or a camel, or a dog, or a cat. Most preferably, the mammalian subject is a human.

The patient may exhibit signs or symptoms that suggest that they have cancer. By “exhibit”, we include that the subject displays a cancer symptom and/or a cancer diagnostic marker, and/or the cancer symptom and/or a cancer diagnostic marker can be measured, and/or assessed, and/or quantified.

It would be readily apparent to the person skilled in medicine what the cancer symptoms and cancer diagnostic markers would be and how to measure and/or assess and/or quantify whether there is a reduction or increase in the severity of the cancer symptoms, or a reduction or increase in the cancer diagnostic markers; as well as how those cancer symptoms and/or cancer diagnostic markers could be used to form a prognosis for the cancer.

Cancer treatments are often administered as a course of treatment, which is to say that the therapeutic agent is administered over a period of time. The length of time of the course of treatment will depend on a number of factors, which could include the type of therapeutic agent being administered, the type of cancer being treated, the severity of the cancer being treated, and the age and health of the subject, amongst others reasons.

By “during the treatment”, we include that the subject is currently receiving a course of treatment, and/or receiving a therapeutic agent, and/or receiving a course of a therapeutic agent.

As discussed above, the combination of an oncolytic virus capable of expressing a first antibody molecule that specifically binds to CTLA-4, and a second antibody molecule that specifically binds to PD-1 and/or PD-L1, is specifically for use in the treatment of cancer in a patient wherein the cancer comprises or consists of a cold tumor.

Therefore, in some embodiments, the cold tumor is treated by the first and second antibody molecule.

The skilled person will appreciate that determining whether the cold tumor has been “treated” involves the same kind of determination used for any other type of tumor. For example, the skilled person will look for signs such as tumor shrinkage (in both injected and uninjected tumors) which can be measured using a CT scan, and/or progression-free survival, and/or overall survival. In other cases, this may be a more subjective effect, such as a reduction in severity of symptoms reported by the subject. The measurement of therapeutic effects in subjects in response to the administration of therapeutic antibodies is well known in the art.

“Cold tumors” refer to tumors that are poorly infiltrated by inflammatory immune cells, most notably T cells and in particular CD8⁺ T cells. Cold tumors are highly clinically relevant, since tumor immune infiltration and in particular CD8+ T cell infiltration has been widely demonstrated to correlate with longer disease-free survival (DFS) and/or overall survival (OS) in cancers with different histological features and anatomical location. This has been demonstrated in both primary and metastatic settings (Bruni et al., 2020) including melanoma, most squamous cell carcinomas (SCCs), large cell lung cancer and several types of adenocarcinoma (Galon et al., 2006; Fridman et al., 2012; Fridman et al., 2017; Hu et al., 2018) and to correlate with and predict responsiveness to ICB (Galon and Bruni, 2019).

For example, it was recently shown that CD8⁺ T cell densities correlate with response/progression to ICB with antibodies to anti-CTLA-4 and PD-1/PD-L1 in melanoma (Tumeh et al. 2014), renal cell carcinoma (McDermott, Huseni et al. 2018) and NSCLC (Thommen, Koelzer et al. 2018) in human solid cancer patients. Similarly, it is well appreciated that preclinical mouse tumor models differ quantitatively and qualitatively with respect to immune infiltration, and that the B16/C57BL6 model is particularly scarce with respect to immune cell infiltration, including CD8⁺ T cells (Mosely et al. 2017). Further, similar to human “cold tumors” the B16/C57BL6 model is particularly resistant to systemic ICB with anti-CTLA-4 and/or anti-PD-1/L1 making it useful to help identify therapies that help overcome “cold tumor” resistance to systemic ICB.

Clinically relevant assays that quantify tumor cell, immune cell or composite cell levels of PD-L1 have been devised and are used in the clinic to help identify patients to treat with anti-PD-1/L1 ICB reagents e.g. pembrolizumab (see prescribing information for KEYTRUDA® Section 2.1, which describes how patients are selected for therapy—available at https://www.accessdata.fda.gov/drugsatfda_docs/label/2021/125514s096lbl.pdf).

Recent data, based on multicolour immune fluorescence, identified invasive margin CD8⁺ T cell density as the best full predictive parameter, and tumor CD8⁺ T cell density as the second-best predictor of response/progression to PD-1 blocking therapy in melanoma (Tumeh et al., 2014). Further, principal component analysis demonstrated that CD8 infiltration, PD-1 and PD-L1 significantly correlated with treatment outcome. These data support that CD8⁺ T cell density may be a useful way to identify patients with cold tumors. Recently, an immune based assay named the “Immunoscore” was developed to quantify in situ the CD3⁺ CD8⁺ T cell infiltrate in tumors of Cancer patients (Bruni et al., 2020; Galon et al., 2006; Lanzi et al., 2020).

The Immunoscore is an immunohistochemistry and digital pathology-based scoring system assessing the densities of CD3⁺ and CD8⁺ T cells in the tumor and its invasive margin. Briefly, two adjacent slides of formalin-fixed, paraffin-embedded tumor blocks are stained with anti-CD3 and anti-CD8 antibodies in an autostainer. The slides are then scanned and the digital images are used to quantify the densities of the cells of interest with a digital pathology software. The densities are finally translated into an Immunoscore, ranging from Low Immunoscore (I0) to High Immunoscore (I4).

An approach that aligns immunoscore to the concept of “hot and cold tumors” has been proposed by Galon and Bruni (Galon et al., 2019; Angell et al., 2020). Using this approach, tumors were classified into four categories based on T cell infiltration: hot immune tumors; altered immunosuppressed immune tumors; altered-excluded immune tumors; and cold tumors.

The characteristics of these four types of tumors are described in detail in Box 1 of Galon et al., 2019, which describes the tumor types according to the following characteristics:
1. Hot immune tumors (also referred to herein as hot tumors)
   High degree of T cell and cytotoxic T cell infiltration (high immunoscore)
   Checkpoint activation (programmed cell death protein 1 (PD-1), cytotoxic T lymphocyte-associated antigen 4 (CTLA-4), T cell immunoglobulin mucin receptor 3 (TIM-3) and lymphocyte activation gene 3 (LAG-3)) or otherwise impaired T cell functions (for example, extracellular potassium-driven T cell suppression)
2. Altered immunosuppressed immune tumors
   Poor, albeit not absent, T cell and cytotoxic T cell infiltration (intermediate Immunoscore)
   Presence of soluble inhibitory mediators (transforming growth factor-β (TGFβ), interleukin 10 (IL-10) and vascular endothelial growth factor (VEGF))
   Presence of immune suppressive cells (myeloid-derived suppressor cells and regulatory T cells)
   Presence of T cell checkpoints (PD-1, CTLA-4, TIM-3 and LAG-3)
3. Altered-excluded immune tumors
   No T cell infiltration inside the tumor bed; accumulation of T cells at tumor borders (invasive margin) (intermediate Immunoscore)
   Activation of oncogenic pathways
   Epigenetic regulation and reprogramming of the tumor microenvironment
   Aberrant tumor vasculature and/or stroma
   Hypoxia
4. Cold immune tumors (also referred to herein as cold tumors)
   Absence of T cells within the tumor and at the tumor edges (low Immunoscore)
   Failed T cell priming (low tumor mutational burden, poor antigen presentation and intrinsic insensitivity to T cell killing).

Therefore, in some embodiments, the patient is considered as having a cold tumor if they have a tumor fitting the definition of an altered immunosuppressed immune tumor, an altered-excluded immune tumor, or a cold immune tumor as defined above.

It will be appreciated that each of the above types of tumor will have a different level of response to T cell checkpoint inhibition, with the cold immune tumors having the lowest level of response (absent response), followed by the altered-excluded, and the altered immunosuppressed immune tumors, respectively (which have a sub-optimal level of response).

The herein used definition of “cold tumors” i.e. tumors poorly infiltrated by immune cells (e.g. CD3+ and CD8+ T cells) encompasses both classical cold immune, altered-excluded, and altered-immunosuppressed tumors, since all of these categories are defined by poor (immune altered) or absent (immune excluded and cold) T cell infiltration. This is the equivalent of cancer patients with Immunoscores I, II or III but not IV (the latter being T cell inflamed tumors). As such, the current invention is applicable to patients with immunoscore I, II and III, but not IV.

Therefore, in some embodiments, the patient is considered as having a cold tumor if the tumor has an Immunoscore of I, or II, or III.

A person skilled in the art will appreciate that other assays and technologies than the immunoscore or T cell density may help identify particularly resistant “cold types” of cancer.

In some embodiments, a cold tumor has anergized (or anergic) lymphocytes. By this we mean that the lymphocytes fail to respond to antigen. Methods of determining anergic lymphocytes are well known in the art.

In some other embodiments, a cold tumor has a low level of CD3 positive cells. For instance the cold tumor may have less than 10% CD3 positive cells (as a percentage of the total cells in the tumor), i.e. less than 5%, less than 4%, less than 3%, less than 2%, less than 1%, less than 0.5%, less than 0.1%, or no CD3 positive cells. Methods of measuring the percentage of CD3 positive cells is known in the art.

As discussed above, a cold tumor as described herein is a tumor that is not typically well targeted by the immune system. In some alternative or additional embodiments, such cold tumors may also be classified into the following types:
Immune deserted tumors, i.e. there is a total lack of immune response in the tumor due to a lack of tumor-infiltrating T cells.
Immune excluded tumors, i.e. responsive T cells are generated but are unable to penetrate the tumor to mount a response against it, T cells may be present at the tumor periphery.
Tumors with poor immune infiltration, i.e. the level of penetration of immune cells (T cells) into the tumor microenvironment is reduced.

By “cold tumor” as used herein, we also include all of immune deserted tumors, immune excluded tumors, and tumors with poor immune infiltration. These definitions may be used alternatively, or in addition to, the definitions of a cold tumor (as an altered-immunosuppressed, altered-excluded, or cold immune tumor) discussed above. In some embodiments, the altered immunosuppressed immune tumor corresponds to the tumors with poor immune infiltration. In some embodiments, the altered-excluded immune tumors correspond to the immune excluded tumors. In some embodiments, the cold immune tumors correspond to the immune deserted tumors.

By “comprises or consists of a cold tumor” we refer to cancers that may be made up of cold tumors and non-cold tumors. For example, this may occur if the original cancer (which may be a cold tumor) has metastasized and formed secondary tumors that are not cold tumors. The patient herein may have multiple tumors, only one of which needs to fulfil the requirements of a cold tumor for the present invention to be beneficial. In other embodiments, the patient may have a single tumor that is considered a cold tumor.

Cancers that may fall into these “cold tumor” subtypes include, but are not limited, to the following: melanoma, pancreatic cancer, prostate cancer, colorectal cancer, hepatocellular carcinoma, lung cancer, bladder cancer, kidney cancer, gastric cancer, cervical cancer, Merkel cell carcinoma, ovarian cancer, head and neck cancer, mesothelioma or breast cancer.

Each one of the above-described cancers is well-known, and the symptoms and cancer diagnostic markers are well described, as are the therapeutic agents used to treat those cancers. Accordingly, the symptoms, cancer diagnostic markers, and therapeutic agents used to treat the above-mentioned cancer types would be known to those skilled in medicine.

Clinical definitions of the diagnosis, prognosis and progression of a large number of cancers rely on certain classifications known as staging. Those staging systems act to collate a number of different cancer diagnostic markers and cancer symptoms to provide a summary of the diagnosis, and/or prognosis, and/or progression of the cancer. It would be known to the person skilled in oncology how to assess the diagnosis, and/or prognosis, and/or progression of the cancer using a staging system, and which cancer diagnostic markers and cancer symptoms should be used to do so.

By “cancer staging”, we include the Rai staging, which includes stage 0, stage I, stage II, stage III and stage IV, and/or the Binet staging, which includes stage A, stage B and stage C, and/or the Ann Arbour staging, which includes stage I, stage II, stage III and stage IV.

It is known that cancer can cause abnormalities in the morphology of cells. These abnormalities often reproducibly occur in certain cancers, which means that examining these changes in morphology (otherwise known as histological examination) can be used in the diagnosis or prognosis of cancer. Techniques for visualizing samples to examine the morphology of cells, and preparing samples for visualization, are well known in the art; for example, light microscopy or confocal microscopy.

By “histological examination”, we include the presence of small, mature lymphocyte, and/or the presence of small, mature lymphocytes with a narrow border of cytoplasm, the presence of small, mature lymphocytes with a dense nucleus lacking discernible nucleoli, and/or the presence of small, mature lymphocytes with a narrow border of cytoplasm, and with a dense nucleus lacking discernible nucleoli, and/or the presence of atypical cells, and/or cleaved cells, and/or prolymphocytes.

It is well known that cancer is a result of mutations in the DNA of the cell, which can lead to the cell avoiding cell death or uncontrollably proliferating. Therefore, examining these mutations (also known as cytogenetic examination) can be a useful tool for assessing the diagnosis and/or prognosis of a cancer. An example of this is the deletion of the chromosomal location 13q14.1 which is characteristic of chronic lymphocytic leukaemia. Techniques for examining mutations in cells are well known in the art; for example, fluorescence in situ hybridization (FISH).

By “cytogenetic examination”, we include the examination of the DNA in a cell, and, in particular the chromosomes. Cytogenetic examination can be used to identify changes in DNA which may be associated with the presence of a refractory cancer and/or relapsed cancer. Such may include: deletions in the long arm of chromosome 13, and/or the deletion of chromosomal location 13q14.1, and/or trisomy of chromosome 12, and/or deletions in the long arm of chromosome 12, and/or deletions in the long arm of chromosome 11, and/or the deletion of 11q, and/or deletions in the long arm of chromosome 6, and/or the deletion of 6q, and/or deletions in the short arm of chromosome 17, and/or the deletion of 17p, and/or the t(11:14) translocation, and/or the (q13:q32) translocation, and/or antigen gene receptor r rearrangements, and/or BCL2 rearrangements, and/or BCL6 rearrangements, and/or t(14:18) translocations, and/or t(11:14) translocations, and/or (q13:q32) translocations, and/or (3:v) translocations, and/or (8:14) translocations, and/or (8:v) translocations, and/or t(11:14) and (q13:q32) translocations.

It is known that subjects with cancer exhibit certain physical symptoms, which are often as a result of the burden of the cancer on the body. Those symptoms often reoccur in the same cancer, and so can be characteristic of the diagnosis, and/or prognosis, and/or progression of the disease. A person skilled in medicine would understand which physical symptoms are associated with which cancers, and how assessing those physical systems can correlate to the diagnosis, and/or prognosis, and/or progression of the disease. By “physical symptoms”, we include hepatomegaly, and/or splenomegaly.

Patients with “cold” tumors are unlikely to respond to traditional immune checkpoint blockade therapy (e.g. administration of an anti-CTLA-4 antibody or an anti-PD-1 antibody, for example). Therefore, in some embodiments, patients with cold tumors are resistant to immune checkpoint blockade therapy.

In light of these observations, resistance to ICB constitutes a significant unmet medical need and drugs that could help overcome resistance hold great therapeutic promise. Therefore, the advantage of the present invention is that the oncolytic virus capable of expressing a first antibody that specifically binds to CTLA-4, in combination with an antibody specific to PD-1 and/or PD-L1, produces a synergistic effect that is capable of overcoming said resistance, and targets cold tumors that were previously not treatable using immune checkpoint inhibitors.

The invention also encompasses pharmaceutical compositions comprising the combination of the oncolytic virus capable of expressing a first antibody molecule that specifically binds to CTLA-4, and a second antibody molecule that specifically binds to PD-1 and/or PD-L1, in combination with a pharmaceutically acceptable carrier and/or diluent and/or adjuvant. Such pharmaceutically acceptable carriers, diluents and adjuvants are known in the art.

The antibody molecules, nucleotide sequences, plasmids, viruses, cells and/or pharmaceutical compositions described herein may be suitable for parenteral administration including aqueous and/or non-aqueous sterile injection solutions which may contain anti-oxidants, and/or buffers, and/or bacteriostats, and/or solutes which render the formulation isotonic with the blood of the intended recipient; and/or aqueous and/or non-aqueous sterile suspensions which may include suspending agents and/or thickening agents. The antibody molecules, nucleotide sequences, plasmids, cells and/or pharmaceutical compositions described herein may be presented in unit-dose or multi-dose containers, for example sealed ampoules and vials, and may be stored in a freeze-dried (i.e. lyophilised) condition requiring only the addition of the sterile liquid carrier, for example water for injections, immediately prior to use.

Extemporaneous injection solutions and suspensions may be prepared from sterile powders, and/or granules, and/or tablets of the kind previously described.

For parenteral administration to human patients, the daily dosage level of the anti-PD-1 and/or anti-PD-L1 antibody molecule will usually be from 1 mg/kg bodyweight of the patient to 20 mg/kg, or in some cases even up to 100 mg/kg administered in single or divided doses. In some preferred embodiments, the dose is 10 mg/kg. Lower doses may be used in special circumstances, for example in combination with prolonged administration. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

Typically, a pharmaceutical composition (or medicament) described herein comprising an antibody molecule will contain the anti-PD-1 and/or anti-PD-L1 antibody molecule at a concentration of between approximately 2 mg/ml and 150 mg/ml or between approximately 2 mg/ml and 200 mg/ml. In some embodiments, the pharmaceutical compositions will contain the anti-PD-1 and/or anti-PD-L1 antibody molecule at a concentration of 10 mg/ml or 25 mg/ml.

In some embodiments, when the anti-PD-1 antibody is pembrolizumab, the antibody is used at a dose of approximately 25 mg/ml. In some other embodiments, pembrolizumab is used at a dose of 200 mg (iv) every 3 weeks or at a dose of 400 mg (iv) every 6 weeks.

In some embodiments, when the anti-PD-1 antibody is nivolumab, the antibody is used at a dose of approximately 10 mg/ml. In some embodiments, nivolumab is used at a dose of 240 mg (iv) every 2 weeks or at a dose of 480 mg (iv) every 4 weeks. In some embodiments, nivolumab may be used in combination with the anti-CTLA-4 antibody ipilimumab, in which case nivolumab is used at a dose of 1 mg/kg every 3 weeks for a maximum of 4 doses or 3 mg/kg every 2 or 3 weeks.

In some embodiments, when the anti-PD-L1 antibody is atezolizumab, the antibody is used at a dose of approximately 60 mg/ml. In some other embodiments, atezolizumab is used at a dose of 840 mg (iv) every 2 weeks or at a dose of 1200 mg (iv) every 3 weeks or at a dose of 1680 mg (iv) every 4 weeks.

The skilled person will appreciate that any of the anti-PD-1 or anti-PD-L1 antibodies described herein can be used at any dose or dosage regimen described in their prescribing information.

Typically, a pharmaceutical composition (or medicament) will contain the oncolytic virus described herein at a concentration of between approximately 10³ to 10¹² vp (viral particles), iu (infectious unit) or pfu (plaque-forming units) depending on the virus and quantitative technique. The quantity of pfu present in a sample can be determined by counting the number of plaques following infection of permissive cells (e.g. CEF or Vero cells) to obtain a plaque forming units (pfu) titer, the quantity of vp by measuring the 260 nm absorbance, and the quantity of iu by quantitative immunofluorescence, e.g. using anti-virus antibodies. As a general guidance, individual doses which are suitable for a pharmaceutical composition comprising an oncolytic poxvirus range from approximately 10³ to approximately 10¹⁰ pfu, advantageously from approximately 10³ pfu to approximately 10⁹ pfu, preferably from approximately 10⁴ pfu to approximately 10⁷ pfu; and more preferably from approximately 10⁶ pfu to approximately 10⁷ pfu.

In some embodiments, when the subject is a mouse, the optimal dose of an oncolytic virus is approximately 10⁶ to 10⁷ pfu. In some embodiments, when the subject is a human, the optimal dose of an oncolytic virus is approximately 10⁶ to 10⁹ pfu.

Generally, in humans, oral or parenteral administration of the antibody molecules, nucleotide sequences, plasmids, viruses, cells and/or pharmaceutical compositions described herein is the preferred route, being the most convenient. For veterinary use, the antibody molecules, nucleotide sequences, plasmids, viruses, cells and/or pharmaceutical compositions described herein are administered as a suitably acceptable formulation in accordance with normal veterinary practice and the veterinary surgeon will determine the dosing regimen and route of administration which will be most appropriate for a particular animal. Thus, the present invention provides a pharmaceutical formulation comprising an amount of an antibody molecule, nucleotide sequences plasmid, virus and/or cell of the invention effective to treat various conditions (as described above and further below). Preferably, the antibody molecules, nucleotide sequences, plasmids, viruses, cells and/or pharmaceutical compositions described herein is adapted for delivery by a route selected from the group comprising: intravenous; intratumoral; intramuscular; subcutaneous. Administration can be in the form of a single injection or several repeated injections (e.g. with the same or different doses, with the same or different routes, at the same or different sites of administration). For illustrative purposes, individual doses comprising approximately 10⁴, 5×10⁴, 10⁵, 5×10⁵, 10⁶, 5×10⁶, 10⁷, 5×10⁷, 10⁸, 5×10⁸, 10⁹, 5×10⁹ or 10¹⁰ pfu of an oncolytic poxvirus (e.g. the TK and RR-defective vaccinia virus described herein) are particularly suited for intratumoral administration.

The present invention also includes antibody molecules, nucleotide sequences, plasmids, viruses, cells and/or pharmaceutical compositions described herein comprising pharmaceutically acceptable acid or base addition salts of the polypeptide binding moieties of the present invention. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others. Pharmaceutically acceptable base addition salts may also be used to produce pharmaceutically acceptable salt forms of the agents according to the present invention. The chemical bases that may be used as reagents to prepare pharmaceutically acceptable base salts of the present agents that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others. The antibody molecules, nucleotide sequences, plasmids, viruses and/or cells described herein may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilised (freeze dried) polypeptide binding moiety loses no more than about 20%, or no more than about 25%, or no more than about 30%, or no more than about 35%, or no more than about 40%, or no more than about 45%, or no more than about 50% of its activity (prior to lyophilisation) when re-hydrated.

In some embodiments, the viral composition is suitably buffered at a physiological or slightly basic pH (e.g. from approximately pH 7 to approximately pH 9 with a specific preference for a pH comprised between 7 and 8.5 and more particularly close to 8). It might be beneficial to also include in the viral composition a monovalent salt so as to ensure an appropriate osmotic pressure. Said monovalent salt may notably be selected from NaCl and KCl, preferably said monovalent salt is NaCl, preferably in a concentration of 10 to 500 mM (e.g 50 mM). A suitable viral composition comprises saccharose 50 g/L, NaCl 50 mM, Tris-HCl 10 mM and Sodium glutamate 10 mM, pH8. The composition may also be formulated so as to include a cryoprotectant for protecting the oncolytic virus at low storage temperature. Suitable cryoprotectants include without limitation sucrose (or saccharose), trehalose, maltose, lactose, mannitol, sorbitol and glycerol, preferably in a concentration of 0.5 to 20% (weight in g/volume in L, referred to as w/v) as well as high molecular weight polymers such as dextran or polyvinylpyrrolidone (PVP).

It will be appreciated that the compositions comprising the oncolytic virus capable of expressing the first antibody molecule, and the compositions comprising the second antibody molecule as described herein, may be administered as a single composition at the same time (i.e. simultaneously).

Alternatively, these compositions may be administered separately, either at similar times or at different time points (e.g. a day or week apart). For example, the oncolytic virus may be administered before the second antibody molecule. In other examples the second antibody molecule may be administered before the oncolytic virus. Such sequential administration may be achieved by temporal separation of the oncolytic virus and second antibody molecule. Alternatively, or in combination with the first option, the sequential administration may also be achieved by spatial separation of the oncolytic virus and second antibody molecule, by administration of the oncolytic virus that is capable of expressing an anti-CTLA-4 antibody in a way, such as intratumoral, so that it reaches the cancer prior to the second antibody molecule, which is then administered in a way, such as systemically, so that it reaches the cancer after the oncolytic virus.

The oncolytic virus and the second antibody molecule of the present invention can be administered according to the established treatment regimen of each component, as described above. This means that the administration of each component may be at the same time, or at different times relative to one another.

In some embodiments, the administration of the oncolytic virus capable of expressing the first antibody molecule, and the second antibody molecule as described herein, may be repeated. For example, the administration may be repeated twice, three times, four times, five times, or as many times as is necessary for there to be a therapeutic effect.

Preferred, non-limiting examples which embody certain aspects of the invention will now be described, with reference to the following drawings and examples:

### DESCRIPTION OF THE DRAWINGS

FIG. 1: Biochemical and functional characterization of novel Treg-depleting αCTLA-4 mAbs. (A) Antibody mediated survival and (B) TIL modulation in CT26 tumor-bearing BALB/c mice. Animals with established tumors received four injections (10 mg/kg) of antibodies with indicated Treg-associated specificity or control mIgG2a antibody (n=5-15). (C) CTLA-4 specific mAbs induce ADCC of in vitro-activated CD4⁺ T cell. Lysed target T cells were identified by FACS. Figure shows mean±SD (n=4-8) **p<0.01 by Student's t-test. (D) Anti-CTLA-4 (IgG1) mAbs mediate Treg depletion in vivo in huPBMC mice. Clone 4-E03 shows enhanced depletion of human Treg cells (upper panel) but not CD8⁺ T cells (lower panel) compared with ipilimumab. Each dot represents one mouse. Graph shows mean data from 2 experiments. *p<0.05 by one-way ANOVA. (E) 4-E03 hIgG1 and ipilimumab binding to human, mouse and cynomolgus CTLA-4 and CD28 by ELISA. (F) 4-E03 IgG1 binding to in vitro-activated CTLA-4 expressing human T cells was pre-blocked with rhCTLA-4-Fc protein (black line) (G) 4-E03 and 2-C06 block CD80 and CD86 binding to CTLA-4 by ELISA. (H) Functional ligand blockade in vitro. Graphs show IL-2 in supernatants following treatment of in vitro activated human PBMCs with αCTLA-4. A representative donor is shown (n=6). (I) Dose-dependent binding of mouse αCTLA-4 mAb to mCTLA-4-transfected cells by flow-cytometry. (J) Anti-CTLA-4 mAbs block B7 ligand (CD80 and CD86) binding to recombinant CTLA-4 by ELISA. (K) Treg depleting activity and CD8⁺ T cell/Treg ratios (n=4-8) and (L) survival induced by αmouse CTLA-4 antibodies in CT26 tumor bearing BALB/c mice.

FIG. 2: Generation and characterization of oncolytic Vaccinia viruses expressing Treg-depleting αCTLA-4 and GM-CSF. (A) Schematic illustration of the Vaccinia virus vectors used to encode heavy (at J2R locus) and light chains of the αCTLA4 antibody and GM-CSF (at the I4L locus). (B) Replication kinetics in LoVo cells and (C) oncolytic activity on MIA PaCa-2 cells of VV_{GM}-αhCTLA4 (BT-001). TG6002 (recombinant J2R and I4L deleted Vaccinia virus) was added as control. (D) Electrophoresis profile after Coomassie blue staining of 4-E03 purified from MIA PaCa-2 BT-001-infected cell cultures. Lanes 2 and 5: recombinant 4-E03; lanes 1 and 4:4-E03 purified from culture medium of MIA PaCa-2 BT-001-infected cells. Lanes 1 and 2: non-reducing conditions; Lanes 3 and 4: reducing conditions. (E) Expression levels of 4-E03 and human GM-CSF of indicated human tumor cells 48 h post-infection by VV_{GM}-αhCTLA4 (BT-001). (F) Biological activity of GM-CSF produced in E) determined by TF-1 proliferation assay. Recombinant human GM-CSF (Molgramostim obtained from European Pharmacopoeia Reference Standard) was included as positive control. (G and H) Functional assessment of 4-E03 produced by BT-001-infected MIA PaCa-2 cells (G) in vitro: by binding to immobilized recombinant hCTLA protein as in FIG. 1E and (H) in vivo: (Treg depletion) as in FIG. 1D.

FIG. 3: Intratumoral VV_{GM}-αCTLA4 has in vivo anti-tumor activity associated with tumor-restricted CTLA-4 receptor saturation and Treg depletion. (A) CT26 tumor-bearing mice were treated with VV_{GM}-αCTLA4 (7.5×10⁶, 7.5×10⁵, or 7.5×10⁴ pfu), VV-αCTLA4 (7.5×10⁶ pfu) or empty control VV (7.5×10⁶ pfu) (n=20 mice/group). (B) Pharmacokinetics of αCTLA-4 in tumors and serum of CT26 tumor-bearing mice after three i.t. injections (days 0, 2, and 4) of VV_{GM}-αCTLA4 at 10⁷ pfu or after single i.p. injection of 3 mg/kg of αCTLA-4 mAb 5-B07 (n=3 mice/time point). Area in grey indicates EC₁₀ to EC₉₀ range of CTLA-4 receptor saturation (see FIG. 1J). (C) Numbers of FoxP3⁺ cells were analysed by FACS in tumors and spleen at day 10 post VV_{GM}-αCTLA4 injection. Graphs show pooled data from 3 independent experiments (n=13 mice/group).

FIG. 4: Intratumoral VV_{GM}-αCTLA4 has broad anti-tumor activity in syngeneic tumor models spanning inflamed and cold tumor microenvironments. (A) BALB/c mice bearing CT26, A20 or EMT6 tumors, or C57BL/6 mice bearing MC38 or B16 tumors, received three i.t. injections of VV_{GM}-αCTLA4 or control virus lacking amCTLA-4 mAb (VV empty or VV_{GM}). Treatment started when tumors had a volume of ˜50-100 mm³ or 4 days after tumor cell injection (B16 only). Graphs show tumor growth of individual mice and corresponding survival (n=10). (B) CT26 tumor cells were implanted into the right and left flanks of BALB/c mice. i.t. injections (vertical dotted lines, same as in A)) in right flank tumors with VV_{GM}-αCTLA4 started when tumors reached a volume of ˜100 mm³ (n=9-10).

FIG. 5: Intratumoral VV_{GM}-αCTLA4 elicits robust systemic CD8⁺ T cell dependent anti-tumor immunity. (A) BALB/c mice were treated with CD8 (short-dashed line) or CD4 (long-dashed line) depleting antibody pre and post s.c. challenge with CT26 tumor cells. When tumors reached a volume of ˜20-50 mm³, treatment as in FIG. 4A commenced. One representative experiment (out of 2) is shown with 10 mice per group. (B-D) CT26 tumor-bearing mice were treated i.t. with VVs or i.p. with αCTLA-4 mAb (clone 5-B07 at 3 mg/kg). Tumor cell suspensions and splenocytes were restimulated ex vivo with VV- or CT26 (AH-1)-specific peptide and the percentage of IFN-γ⁺ and TNF-α⁺ CD8⁺ T cells, or MHC class I-labeled multimer positive CD8⁺ T cells was quantified by FACS. (B) shows flow-cytometry dot plots of AH-1 peptide positive (upper panel) or cytokine positive (lower panel) splenocytes. Quantification of (C) antigen-specific and (D) IFN-γ⁺/TNF-α⁺ CD8⁺ T cells in indicated organs. Each dot represents one mouse. (n=3-6 experiments) *p<0.05, **p<0.01, ***p<0.005, ****p<0.001 by one-way ANOVA.

FIG. 6: Intratumorally induced CD8⁺ T cell anti-tumor immunity is FcγR-dependent and cDC1-dependent. (A) CT26 tumor-bearing WT and Fcer1g^{-/-} BALB/c mice received i.t. injections of VV_{GM}-αCTLA4 or PBS as in FIG. 4A. Graphs show tumor volume (left and center panels) and mouse survival (right panel). Vertical lines indicate end of the treatment. (n=10 mice/group) (B) GO terms enriched in the set of 352 differentially expressed genes, either up or down-regulated, in CT26 tumors treated with VV_{GM}-αCTLA4 versus VV empty. The 20 enriched terms with lowest adjusted p-value are shown. (C) Network view of the differentially expressed genes associated with the 5 most enriched GO terms from FIG. 6b. Only genes up-regulated were found associated with these 5 enriched GO terms. (D) MC38 tumor-bearing WT and Batf3^{-/-} BALB/c mice received i.t. injections of VV_{GM}-αCTLA4 or PBS as in FIGS. 4A and 6A. Graphs show tumor volume (left and center panels) and mouse survival (right panel). Vertical lines indicate end of the treatment. (n=8-10 mice/group).

FIG. 7: Intratumoral VV_{GM}-αCTLA4 expands peripheral effector CD8⁺ T cells and reduces Treg and exhausted CD8⁺ T cells. CT26 “twin” tumor-bearing BALB/c mice were treated i.t. (right flank tumors only) with VV_{GM}-αCTLA4 or PBS. Spleens, injected and contralateral tumors were collected on day 10 post treatment and stained with a high-dimensional panel designed to identify T cell populations. (A) i.t. VV_{GM}-αCTLA4 reduced activated CD4⁺ Treg cells (FoxP3⁺ Klrg1⁺, “T1”), reduced exhausted CD8⁺ T cells (PD1⁺ TIM3⁺, “T2”), and expanded activated effector CD8⁺ T cells (Klrg1⁺, “T3”) in injected and uninjected tumors (upper panel) and expanded activated CD8⁺ T cells in spleen (S1, lower panel) (B) shows quantification of data illustrated in A. One representative experiment (out of 3) with 5 mice/group is shown. (C) Flow cytometry plots show characteristic markers of selected i.t. T cell clusters.

FIG. 8: Intratumoral VV_{GM}-αCTLA4 synergizes with αPD-1 to reject “cold” ICB-resistant tumors. (A and B) C57BL/6 mice carrying two B16 tumors, one large (5×10⁵ cells, treated tumor) and one small tumor (1×10⁵ cells, contralateral side) received three i.t. injections with VV_{GM}-αCTLA4 (vertical dotted lines) and i.p. αPD-1 (29F.1A12, 10 mg/kg; twice a week for three weeks, grey area). (A) Survival (n=10-20), *p<0.05 by Log-rank test. (B) Tumor growth curves of intratumorally injected and contralateral tumors. (C) A20 tumor-bearing BALB/c mice were treated thrice with VV_{GM}-αCTLA4 i.t. (at a sub-optimal dose of 1×10⁵ pfu), αPD-1 i.p. (RMP1-14, full dose of 10 mg/kg) or the combination of both when tumors had reached a volume of ˜135 mm³. Graph shows animal survival (n=10).

FIG. 9: Characterization of CTLA4-specific mAbs. (A) Samples of freshly excised ovarian tumors, ascites, and blood were compared to healthy PBMCs. CTLA-4 expression was assessed on CD4⁺ CD25⁺ CD127-Treg cells, CD4⁺ non-Treg cells, and CD8⁺ effector T cells by flow cytometry and compared to expression on human T cells isolated from NOG spleens two weeks after PBMC transfer. Data represent individual patients/donors with n=11 for healthy PBMCs, n=20 for ascites, n=9 for tumor, and n=5 for patient blood. (B) Human PBMCs were injected i.v. into NOG mice. 2-3 weeks post-transplant, spleens were dissected out and cell suspension was injected i.p. into SCID recipients subsequently treated with 10 mg/kg 4-E03 hIgG1, 4-E03 hIgG1 N297Q or isotype control. Cells were collected by i.p. lavage and quantified 24 h post treatment. % cell depletion was normalised to isotype control. Each dot represents one mouse. (C) Binding kinetics of 4-E03 and ipilimumab to soluble human CTLA-4 were evaluated by Biacore analysis. mAbs were captured on the chip by immobilized anti-human Fc and different concentrations of CTLA-4 protein were injected in each cycle. K_{D} values for 4-E03 and ipilimumab were 0.6 nM and 2.7 nM, respectively. (D) Dose-dependent binding of anti-CTLA-4 mAb to CTLA-4 endogenously expressing human T cells by flow-cytometry. (E) Binding of titrated doses of mouse surrogate antibody 5-B07 mIgG2a (from 10 μg/ml, with 3-fold dilution steps) was tested against mouse CTLA-4 and CD28 by ELISA. (F) 1×10⁶ CT26 cells were injected s.c. into BALB/c mice (n=7). When tumors reached a size of approximately 100 mm³, mice received 200 μg (10 mg/kg) of 5-B07 mIgG2a, 5-B07 mIgG1 N297A or isotype control antibody on days 0, 4 and 7. At day 8, tumors were taken out and TIL analysed by FACS. Data shown are mean values (+SD) of one representative experiment with n=7 mice per group.

FIG. 10: Pharmacokinetics of virus and transgenes in tumor and blood. (A and B) Tumor samples described in FIG. 3B were also used to measure the intratumoral concentration of (A) murine GM-CSF and (B) virus load C-E) Pharmacokinetics in LoVo xenografted tumor. LoVo cells were implanted in the right flank of Swiss nude mice. When the tumor volumes reached ˜120 mm³ (defined as DO) mice were treated by a single injection of either 10⁵ pfu of VV_{GM}-hCTLA4 (BT-001) or VV i.t. or 3 mg/kg of 4-E03 monoclonal antibody i.p. Blood and tumors of three mice were collected at each indicated time point. The concentrations of (C) 4-E03, (D) GM-CSF and (E) virus were determined by ELISA and titration on Vero cell respectively. The lines link the median of values of each time point.

FIG. 11: Treatment with i.t. VV_{GM}-αCTLA4 induces long-lasting anti-tumor responses in treated and untreated tumors. (A) CT26 tumor cells were implanted into the right and left flanks of BALB/c mice. I.t. injections (×3, two days apart) in right flank tumors with VV_{GM}-αCTLA4 started when tumors reached a volume of ˜100 mm³. Virus concentration in treated and contralateral tumors was assessed one day post treatment. (n=3 mice per group; mouse 1-3 (M1-3); N.D.=not detected) B-C) CT26 tumor-bearing mice were treated with indicated doses of VV_{GM}-αCTLA4 as in A). Alternatively, 10⁷ or 10⁵ pfu of VV_{GM}-αCTLA4 was given i.v. Individual tumor growth curves (B) and survival curves (C) are shown. (D) Inhibition of rechallenged tumor growth. CT26 tumor cells were implanted s.c. to BALB/c mice. I.t. treatment with VV_{GM}-αCTLA4 or empty control VV was scheduled as in A). As indicated in the figure legend of FIG. 4B, surviving mice were s.c. rechallenged with CT26 or Renca cells 100 days after the last VV injection.

FIG. 12: Intratumoral induced CD8 T cell immunity is FcγR-dependent. WT and Fcer1g−/− BALB/c mice were challenged with 1×10⁶ CT26 cells s.c. When tumors reached approximately 100 mm³, mice received three i.t. injections of VV_{GM}-αCTLA4 or PBS control at day 0, 2 and 5 (10⁷ pfu final dose). At day 8, tumors and spleen were isolated and FoxP3⁺ CD4⁺ cells analysed by FACS.

FIG. 13: Treatment with i.t. VV_{GM}-αCTLA4 is dependent on CD8⁺ T cells. (A) Groups of 10 BALB/c mice were treated, or not, with 1 mg of a CD8- or CD4-depleting antibody (or corresponding isotype control antibodies)₃ days before challenging the mice with 1×10⁶ CT26 cells s.c. Another 4 days later (on day −3 relative to treatment start), 200 μg of the depleting antibodies were administered i.p. Mice were then treated i.t. with VV_{GM}-αCTLA4 on days 0, 2 and 4 at 1×10⁷ pfu. Percentage survival to the humane end-point is shown. Data are representative for 2 independent experiments. (B) CT26 tumor-bearing mice were treated i.t. with VVs or i.p. with anti-CTLA-4 mAb (clone 5-B07 at 3 mg/kg). Tumor cell suspensions were restimulated ex vivo with VV- or CT26 (AH-1)-specific peptide, cultured for 4 h and the number of IFN-γ⁺ and TNF-α⁺ CD8⁺ T cells was quantified by flow-cytometry. Each dot represents one mouse. Representative experiment (n=3-6) **p<0.01, ***p<0.005, ****p<0.001 by one-way ANOVA. (C) and (D) Heatmap displaying median marker expression of (C) 12 intratumoral (D) 10 splenic CD3+ subpopulations identified by unsupervised clustering.

FIG. 14: B16 tumors are refractory to systemic treatment with anti-CTLA-4 plus anti-PD-1. B16 tumor-bearing C57BL/6 mice were treated i.p. with anti-PD-1 at 10 mg/kg or the combination of anti-PD-1 and anti-CTLA-4 (10 mg/kg) at day 4, 7, 11 (A) Data are expressed as tumor volume (mm³) on the days after cell inoculation as indicated; each line represents an individual mouse. (B) Lower panel shows percentage survival to the humane end-point of B16 bearing mice following indicated treatments. (C) B16 tumor-bearing C57BL/6 mice received three i.t. injections with VV_{GM}-αCTLA4 and/or i.p. anti-PD-1 as described in FIG. 8. Six days after the last treatment, tumors were collected and the number of tumor-infiltrating T cells was analyzed by flow cytometry. Each dot represents one mouse. (n=3 experiments). *p<0.05 by one-way ANOVA. the level of T cell infiltration in the highly T cell inflamed CT26 tumor microenvironment is shown for reference (determined in CT26 tumors around day 20 post cell inoculation).

### EXAMPLES

### Materials and Methods

### Cell Lines

Human embryonic kidney cell line 293T, murine melanoma B16-F10, murine colon carcinoma CT26, murine B cell lymphoma A20, murine mammary EMT6 and murine Lewis lung carcinoma cell line (LL/2) were purchased from the American Type Culture Collection (ATCC) and cells stably transfected with human CTLA-4 (293T-CTLA4) from Crown Bio. Cells were cultured in RPMI+ glutamax (CT26) or DMEM+ glutamax (MC38, B16-F10) supplemented with 10% FCS, 10 mM HEPES and 1 mM sodium pyruvate. EMT6 cells were maintained in Waymouth medium supplemented with 15% FCS, 10 mM HEPES and 1 mM sodium pyruvate. The NK-92 cell line expressing hFcγRIIIA-158V together with GFP (purchased from ATCC) was cultured in supplemented α-MEM medium (Binyamin et al., 2008). Primary cells were cultured in R10 medium (RPMI 1640 containing 2 mM glutamine, 1 mM pyruvate, 100 IU/ml penicillin and streptomycin and 10% FBS; GIBCO by Life Technologies). The human colorectal adenocarcinoma cell line LoVo (ATCC), pancreatic tumor cell line MIA PaCa-2 (ATCC) and human gastric carcinoma cell line Hs-746 T (ATCC) were grown in DMEM (Gibco) supplemented with 10% FBS and containing gentamicin at 40 mg/L. The human ovarian tumor cell line SK-OV-3 (ATCC) and human colorectal carcinoma cell line HCT 116 (ATCC) were grown in Mc Coy's 5A medium (ATCC) supplemented with 10% FBS and containing gentamicin at 40 mg/L. The human erythroblast cell line TF-1 (ATCC) was grown in RPMI 1640 (Sigma) supplemented with 10% FBS and containing gentamicin at 40 mg/L+GM-CSF at 2 ng/ml.

### Mice

Mice were maintained in local pathogen-free facilities. For all experiments young adult mice were sex- and age-matched and randomly assigned to experimental groups. All procedures were approved by the local ethical committee for experimental animals (Malmö/Lunds djurförsöksetiska nämnd); at BioInvent under permit numbers 17196/2018 or 2934/2020; or at Transgene APAFIS Nr21622 project 2019072414343465 and performed in accordance with local ethical guidelines. C57BL/6 and BALB/c mice were obtained from Taconic, Janvier or Charles River. Genetically altered strains used were: C.129P2 (B6)-Fcer1gtm1Rav (Fcer1g-KO on BALB/c background and BALB/cAnNTac WT controls) purchased from Taconic; and B6.129S(C)-Batf3tm1Kmm/J (Hildner et al., 2008); Batf3-KO on C57BL/6J background and C57BL/6J WT controls) purchased from Jackson Laboratories.

### Human (Clinical) Samples and Ethics

Ethical approval was obtained by the Ethics Committee of Skåne University Hospital. Informed consent was provided in accordance with the Declaration of Helsinki. Patient samples were obtained through the Department of Obstetrics and Gynecology and the Department of Oncology at Skånes University Hospital, Lund, Sweden. Ascitic fluid was assessed as single cell suspension that had been isolated.

### Processing of Human Tissue

Ovarian tumor samples obtained from patients undergoing surgery were cut into small pieces and incubated in R10 with DNase I (Sigma) and Liberase™ (Roche Diagnostics) for 20 min at 37° C. Remaining tissue was mechanically dissociated and, together with the cell suspension, passed through a 70 μm cell strainer. Matched peripheral blood samples were obtained and peripheral blood mononuclear cells were separated using Ficoll-Paque PLUS (Cytiva) by centrifugation over Leucosep tubes (Greiner) at 800×g for 20 minutes. Human buffy coats were obtained from the blood center in the hospital of Halmstad (Sweden) and processed according to standard protocol.

### Antibody Dependent Cellular Cytotoxicity

ADCC assays were performed using a NK-92 cell line stably transfected to express the CD16-158V allele together with GFP. CD4⁺ target T cells were isolated from peripheral blood of healthy donors using CD4⁺ T cell isolation kit (Miltenyi Biotec). Cells were stimulated for 72 hours with CD3/CD28 dynabeads (Life Technologies, Thermo Fisher) to upregulate CTLA-4 and 50 ng/ml recombinant hIL-2 (R&D Systems) at 37° C. Target cells were pre-incubated with mAb at 10 μg/ml for 30 min at 4° C. prior to mixing with NK cells. The cells were incubated for 4 h at a 2:1 effector: target cell ratio. Lysis was determined by flow cytometry. Briefly, at the end of the incubation, the cell suspension was stained with VioGreen-conjugated anti-CD4 (M-T466, Miltenyi Biotec) together with Fixable Viability Dye eFluor780 (eBioscience) for 30 min in the dark at 4° C. and the cells were then analysed by FACS.

### Functional Block In Vitro

For the SEB PBMC assay, total PBMCs from healthy donors were seeded on 96-well plates (1×10⁵ cells/well) and stimulated with 1 μg/ml Staphylococcus enterotoxin B (SEB, Sigma Aldrich) in the presence of titrated doses of anti-CTLA-4 IgGs, ranging from 20-0.625 μg/ml. After 3 days, supernatants were harvested and IL-2 quantified by MSD (Meso Scale Discovery, Rockville, USA) according to manufacturer's instructions.

### In Vitro Binding Assays

CTLA-4 expressing transfected cells were incubated with the concentrations of anti-CTLA-4 mAb indicated at 4° C. for 20 mins prior to washing and staining with an APC-labelled goat anti-human secondary antibody (Jackson ImmunoResearch). No binding was observed to cells transfected with empty vector (not shown).

IgG binding to primary cells was analyzed on isolated, in vitro-activated CD4⁺ T cells. Briefly, human peripheral CD4⁺ T-cells were purified from total PBMCs by negative selection using MACS CD4 T-cell isolation kit (Miltenyi Biotec). CD4⁺ T cells were activated in vitro with CD3/CD28 dynabeads (Life Technologies) plus 50 ng/ml recombinant hIL-2 (R&D Systems) in R10 medium for 3 days to upregulate CTLA-4 expression. In vitro-activated human CD4⁺ T cells were incubated with the indicated concentrations of anti-CTLA-4 mAb together with anti-CD4. Bound anti-CTLA-4 mAb were detected with APC-labelled goat-anti-human IgG. In competitive binding assays, 2 μg/ml Alexa 647-labelled anti-CTLA-4 mAb was mixed with recombinant human or cynomolgus CTLA-4-Fc protein (50 μg/ml; R&D Systems) prior to incubation with CTLA-4 expressing cells. Bound IgG binding was detected by FACS.

### VV Generation and Purification

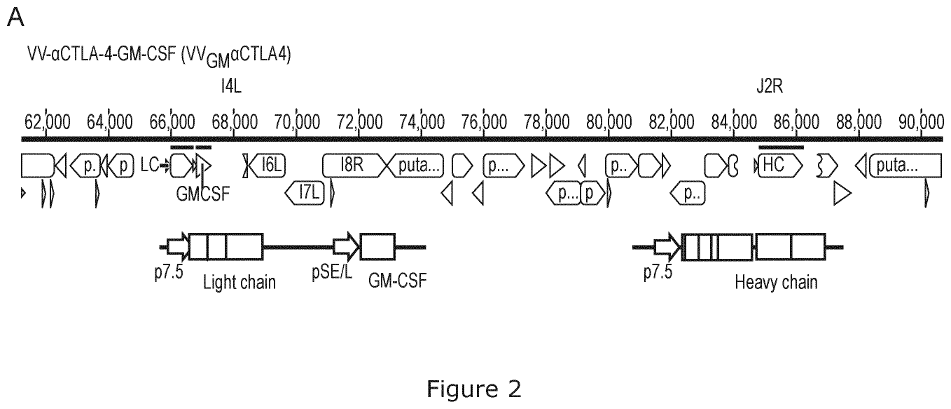

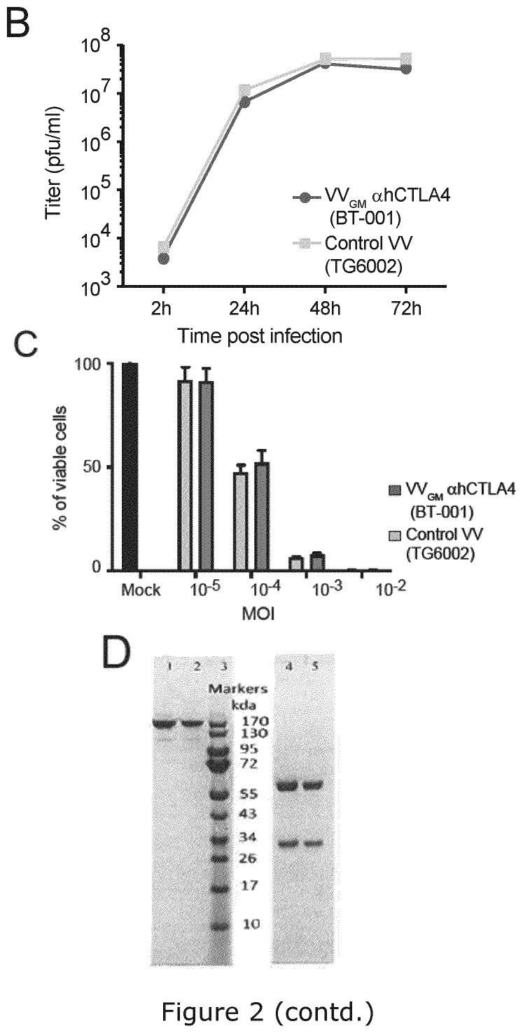

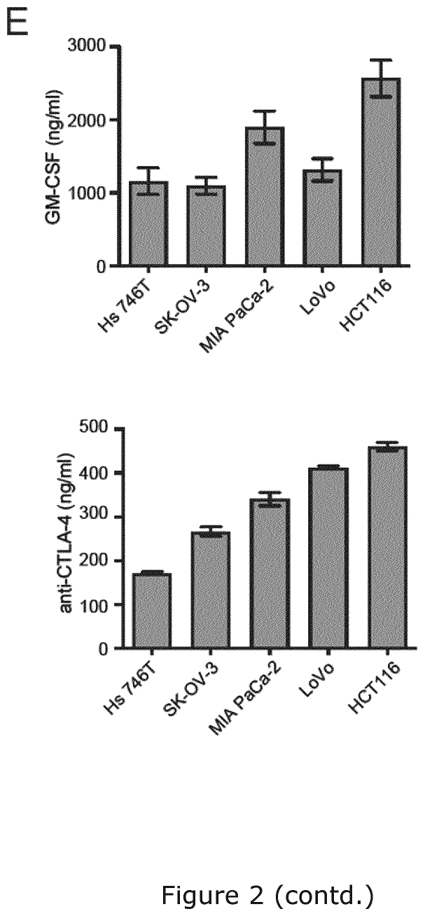

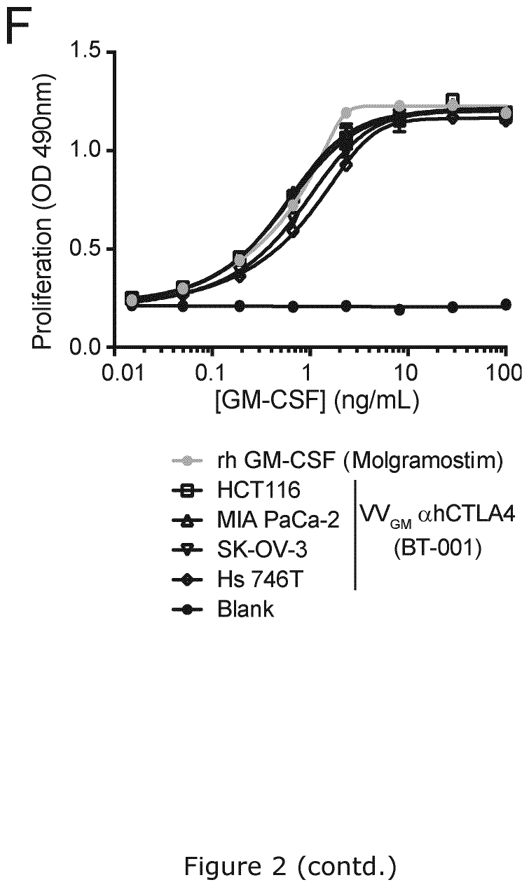

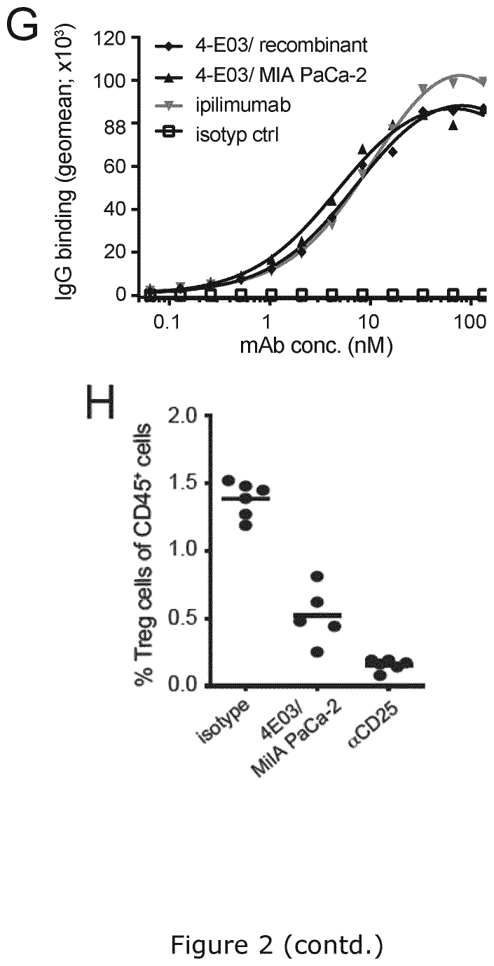

Recombinant viruses were generated by two successive homologous recombination in chicken embryo fibroblast (CEF) using a starting parental Copenhagen vaccinia virus encoding GFP or mCherry at J2R and I4L loci and two transfer plasmids. Transfer plasmids encoded either heavy chain of mAb under the p7.5 promoter and flanked by J2R recombination arms, or the light chain of mAb under the p7.5 promoter in addition, or not, of the murine or human GM-CSF under the pSE/L promoter and flanked by I4L recombination arms (see FIG. 2A). The recombinant viruses were isolated by several cycles of amplification/isolation of non-fluorescent plaques. Recombinant viruses were then produced on CEF and purified after cells lysis by 5 μm filtration followed by purification/concentration using 0.2 μm tangential flow filtration. Finally, the viruses were formulated in saccharose 50 g/L, NaCl 50 mM, Tris 10 mM, Sodium Glutamate 10 mM, pH 8 by diafiltration, aliquoted and stored at −80° C. until use.

All the viruses used for this publication were from the TK—RR-Copenhagen strain:
VV: unarmed vaccinia virus or TG6002 (vaccinia virus encoding FCU1 chimeric enzyme, benchmark recombinant VV)
VV_{GM}: vaccinia virus encoding murine GM-CSF
VV_{GM}-αCTLA4: vaccinia virus encoding murine GM-CSF and 5-B07 (anti-mouse CTLA-4, mouse IgG2a)
VV-αCTLA4: vaccinia virus encoding 5-B07
VV_{GM}-αhCTLA4 (BT-001): vaccinia virus encoding human GM-CSF and 4-E03 (anti-human CTLA-4, human IgG1).

### Virus Replication, Oncolytic Activity and Transgenes Expression In Vitro

Replication of BT-001 was assessed by measuring the total virus titer at 24-, 48- and 72-hours post-infection of LoVo cells with BT-001 at multiplicity of infection (MOI) of 10⁻³ (i.e. 1 virus for 1000 cells). Virus titer was determined by plaque assay on Vero cells.

Oncolytic activity of BT-001 was assessed by quantification of cell viability using cell counter (Vi-Cell) after 5 days of incubation of MIA PaCa-2 cells with BT-001 at the MOI indicated in figure legend. Both replication and oncolytic activity of BT-001 were benchmarked with those of Copenhagen TK-RR-vaccinia virus TG6002 currently under clinical evaluation (Foloppe et al., 2019).

Transgene expression was assessed after infection by BT-001, at MOI 0.05, of several human tumor cell lines: LoVo, HCT 116 (Colon cancer), MIA PaCa-2 (Pancreatic cancer), SK-OV3 (ovarian cancer) and Hs176T (gastric cancer). Culture supernatants were collected 48 hours post-infection, centrifuged and filtered on 0.2 μm prior measurements of 4-E03 and hGM-CSF concentrations by ELISA.

### Antibody Purification

Fifteen F175 flasks containing ˜4.7 10⁷ MIA PaCa-2 cells/flask were infected at MOI 0.01 with BT-001 in DMEM without bovine serum. Seventy-two hours after infection the cells supernatants were harvested, pooled, centrifuged and filtered on 0.2 μm before adding EDTA (2 mM final) and Tris pH 7.5 (20 mM final). The pool supernatant was loaded, at 4° C., on a one mL protA Hitrap column (GE healthcare, ref 17-5079-01) previously equilibrated in PBS. The bound antibodies were eluted by 100 mM glycine HCl pH 2.8 and dialyzed against PBS. Purified 4-E03 was loaded on SDS-PAGE (NuPage Bis-Tris gels 4-12% Thermo NP0323) under reducing or non-reducing conditions and the gel was stained with InstantBlue (Expedeon, ISB1L) Coomassie blue. This purified antibody (i.e. 4-E03 MIA PaCa-2) was further assessed for CTLA-4 binding and in vivo Treg depletion activity.

### Enzyme-Linked Immunosorbent Assays

GMCSF. Human and murine GM-CSF concentrations were determined using the Quantikine® ELISA GM-CSF Immunoassays (R&D Systems).

Human GM-CSF functionality was assessed using the TF-1 proliferation assay. The cellular proliferation of TF-1 cells in presence of known concentrations of hGM-CSF (standard or from BT-001-infected cells) was measured by colorimetry using the enzymatic conversion of MTS to formazan (measured by absorbance at 490 nm) by the dehydrogenases of viable cells. The absorbance at 490 nm was plotted versus the concentration of GM-CSF and the curves compared to the one obtained with recombinant GM-CSF (i.e. Molgramostim).

Binding to CTLA4/CD28 protein. For antibody binding ELISA, purified human CTLA4-Fc, human CD28-Fc (R&D Systems) and mouse CTLA4-Fc (Sino Biologicals) were coated to the assay plate at 1 pmol/well while mouse CD28-His (R&D Systems) was coated at 5 pmol/well. The different antibodies were added at 10 μg/ml and left to bind for 1 h at room temperature. Bound n-CoDeR® mIgG2A or hIgG1 antibodies were detected using either anti-mouse/anti-human H+L-HRP (Jackson Immunoresearch) or Anti-mouse/human Lambda Light Chain Antibody HRP (Bethyl). A chromogenic (TMB T0440) or luminescence substrate (Pierce 37070) was used and plate reading was performed with a Tecan Ultra.

Blocking CD80/CD86 interaction. For ligand blocking ELISA, purified human CTLA4-Fc (R&D Systems) was coated to assay plates at 2 pmol/well (for CD80) or 1 pmol/well (for CD86). Antibodies were added at concentrations ranging from 0.4 pM to 67 nM and left to bind for 1 hour. His-tagged ligands were added at 200 nM and 100 nM, respectively (rhCD80 and rhCD86; R&D Systems) as optimized in a pilot experiment by ELISA (data not shown). The plates were further incubated for 15 minutes. After washing, bound ligand was detected with an HRP-labelled anti-His antibody (R&D Systems). Super Signal ELISA Pico (Thermo Scientific) was used as substrate and the plates were analysed using Tecan Ultra Microplate reader. Alternatively, mouse CTLA4-Fc (Sino Biological) was coated to assay plates at 1 pmol/well. Antibodies were added at a starting concentration of 10 μg/ml (67 nM), with 2-fold dilution steps and left to bind for 1 hour. His-tagged ligands, CD80 and CD86 (Sino Biological), were added at 50 nM and the plates were further incubated for 30 minutes. Detection and reading were performed as described above.

### Mouse Experiments

In vivo tumor experiments. Cultured tumor cells were injected subcutaneously in the left flank only or both flanks (CT26 1×10⁶ cells; MC38 5×10⁵ cells; A20 5×10⁶ cells; EMT6 1×10⁶ cells; B16-F10 0.5-5×10⁵ cells). Unless otherwise specified, mice were treated i.t. with 10⁷ pfu of VV_{GM}-αCTLA4 or control VV, thrice, every other day. For tumor growth experiments, tumor sizes of treated and distant tumors were measured twice a week with a caliper and tumor volume (mm³) was calculated according to the formula: (width²×length×0.52). Animals were euthanized when the total tumor burden (treated and contralateral tumors combined) reached a volume of 2000 mm³ (experimental endpoint). For functional experiments, tissues were collected and processed at the time points indicated in figure legends. Mouse tumors were digested in R10 with DNase I (Sigma) and Liberase™ (Roche Diagnostics) for 15 mins at 37° C. Cells were then passed through a 70 μm cell strainer and used for assays directly. For DC phenotyping, viable leucocytes were enriched following density gradient centrifugation (Cedarline Cat #CL5035).

Primary human xenograft model. PBMC-NOG/SCID mice were generated by intravenously injecting NOG mice (NOD.Cg-Prkdcscid Il2rgtm1Sug/JicTac (Taconic)) with 1-2×10⁷ PBMC isolated using Ficoll-Paque PLUS, in 200 μl PBS. Approximately two weeks after injection, SCID mice (C.B-Igh-1b/IcrTac-Prkdcscid (Taconic)) were subsequently intraperitoneally injected with 10×10⁶ splenocytes from reconstituted NOG mice. 1 h later, mice were treated with 10 mg/kg of mAb. The intraperitoneal fluid of the mice was collected after 24 h. Human T cell subsets were identified and quantified by FACS using following markers: CD45, CD4, CD8, CD25, CD127 (all from BD Biosciences).

Pharmakokinetics of transgenes and virus in tumor and blood. In CT26 tumor model described previously, VV_{GM}-αCTLA4 or VV-αCTLA4 were administrated in same conditions as mentioned above (i.e. 3 i.t. injections of 10⁷ pfu at day 0, 2 and 4). Tumor and blood of three mice/timepoint were collected at day 1, 4 (prior third injection), 8 and 10. Concentrations of virus were measured in whole blood and in tumor homogenized in PBS by viral titration on Vero cells. Concentrations of both 5-B07 and mGM-CSF were measured by ELISA in serum and tumor homogenates. In xenografted human tumor model, LoVo cells were injected subcutaneously in the left flank of Swiss nude mice. After about two weeks when the tumor volume reached ˜120 mm³ the mice were randomized and split in 2 groups (15 mice/groups). First group was injected once i.t. with 10⁵ pfu of VV_{GM}-αhCTLA4 (BT-001) and second group was injected intraperitoneally with 3 mg/kg of 4-E03. Tumor and blood/serum of three mice/timepoint were collected at day 1, 3, 6, 10 and 20 post-virus injection. The virus titer and concentrations of both 4-E03 and hGM-CSF were measured as described in the previous paragraph.

### Antigen-Specific T Cell Responses

Antigen-specific T cell responses were analysed in spleen, treated and contralateral tumors. Briefly, 1×10⁶ isolated cells were restimulated with 2 μg/ml of tumor (AH-1, SPSYVYHQF)- or virus (S9L8, SPGAAGYDL)-specific peptides (BioNordika) (Huang et al., 1996; Russell and Tscharke, 2014). Tumor cells were pulsed for 4 h in the presence of brefeldin A (Sigma). Isolated splenocytes were restimulated for 48 h, the last 4 h in the presence of brefeldin A. Cytokine-producing CD8⁺ T cells were then identified by FACS staining for CD45, TCR-β, CD8, TNF-α, IFN-γ and CD25. In parallel, tumor and virus-specific CD8⁺ T cells were identified using MHC class I multimers (Pentamer H-2Ld-SPGAAGYDL-R-PE (S9L8) ProImmune, Pentamer H-2Ld-TPHPARIGL-R-PE (ctrl) ProImmune, Dextramer H-2Ld-SPSYVYHQF-APC (AH-1) Immudex, Dextramer H-2Ld-TPHPARIGL-APC (ctrl) Immudex).

### Flow Cytometry

Dead cells were routinely identified using the Fixable Viability Dye eFluor™ 780, the Fixable Viability Stain 440UV or propidium iodide and excluded from the analysis along with doublets. Intracellular staining was performed using the FoxP3 Staining Buffer Set (Thermo Fisher Scientific). Sample acquisition was performed on either a BD FACS Verse or Fortessa II and the data were analyzed using FlowJo 10.7.2. To generate the UMAP of intratumoral and splenic CD3⁺ T cells, data were cleaned using the FlowAI tool (v.2.2), samples were then barcoded with treatment group and organ, and concatenated. The FlowJo plugin UMAP (v3.1) was run on the resulting flow cytometry standard (FCS) file using the default settings (distance function: Euclidean, nearest neighbors: 15, and minimum distance: 0.5) and including all the compensated parameters and forward scatter (FSC) and side scatter (SSC) measurements. For cluster identification, the FlowJo plugin x-shift (v1.3) was run on the resulting UMAP using the default settings (nearest neighbors K=82) and including the following parameters: CD4, CD62L, CD25, ICOS, FoxP3, Klrg1, CD44, CTLA-4, PD-1, TIM-3, T-bet, GzmB, Ki-67. Mean expression per cluster for the aforementioned parameters was calculated using scaled channel values obtained from FlowJo. Mean expression heatmaps were generated with parameter means per cluster and scaled between 0 and 1.

### Antibodies

Monoclonal antibodies for flow cytometry: Anti-human CD4-VioGreen (M-T466) Miltenyi Biotec Cat #130-113-259, Anti-human CD25-BV421 (clone M-A251) BD Biosciences Cat #562442, Anti-human CD127-FITC (clone HIL-7R-M21) BD Biosciences Cat #561697, Anti-human CD8-APC (clone RPA-T8) BD Biosciences Cat #555369, Anti-human CTLA-4-PE (clone BNI3) BD Biosciences Cat #555853, mouse IgG2a, k isotype control-PE BD Biosciences Cat #555574, Anti-mouse CD45.2-PerCP-Cy5.5 (clone 104) BD Biosciences Cat #552950, Anti-mouse CD45.2-BUV737 (clone 104) BD Biosciences Cat #612779, Anti-mouse CD25-BV421 (clone 7D4) BD Biosciences Cat #564571, Anti-mouse CD8-BV786 (clone 53-6.7) BD Biosciences Cat #563332, Anti-mouse CD4-BV510 (clone RM4-5) BD Biosciences Cat #563106, Anti-mouse TCRb-Alexa Fluor 488 (clone H57-597) BioLegend Cat #109215, Anti-mouse PD-1-BB700 (clone RMP1-30) BD Biosciences Cat #748242, Anti-mouse CTLA-4-PECF594 (clone UC10-4F10-11) BD Biosciences Cat #564332, Anti-mouse CTLA-4-APC (clone UC10-4B9) BioLegend Cat #106310, Anti-mouse Klrg1-APC (clone 2F1) BD Biosciences Cat #561620, Anti-mouse CD62L-BUV395 (clone MEL-14) BD Biosciences Cat #740218, Anti-mouse TIM3-PE (clone 5D12) BD Biosciences Cat #566346, Anti-mouse ICOS-BV605 (clone 7E.17G9) BD Biosciences Cat #745254, Anti-mouse CD44-APC-Cy7 (clone IM7) BD Biosciences Cat #560568, Anti-mouse Ki67-Alexa Fluor 700 (clone B56) BD Biosciences Cat #561277, Anti-human Granzyme B-R718 (clone GB11) BD Biosciences Cat #566964, Anti-mouse Tbet-BV711 (clone 04-46) BD Biosciences Cat #563320, Anti-mouse FoxP3-PeCy7 (clone FJK-16s) Thermo Fisher Scientific Cat #17-5773-82, Anti-mouse IFNg-PeCy7 (clone XMG1.2) BioLegend Cat #505826, Anti-mouse TNFa-Alexa Fluor 700 (clone MP6-XT22) BD Biosciences Cat #558000, Pentamer H-2Ld-SPGAAGYDL-R-PE (S9L8) ProImmune, Pentamer H-2Ld-TPHPARIGL-R-PE (ctrl) ProImmune, Dextramer H-2Ld-SPSYVYHQF-APC (AH-1) Immudex, Dextramer H-2Ld-TPHPARIGL-APC (ctrl) Immudex.

Secondary antibodies: Goat Anti-Mouse IgG (H+L) Peroxidase Jackson ImmunoResearch Cat #115-035-003, Goat Anti-Human IgG (H+L) Peroxidase Jackson ImmunoResearch Cat #109-035-003, Goat Anti-Human IgG, Fc-Fragment Specific-APC Jackson ImmunoResearch Cat #109-136-098, Goat Anti-Human IgG-APC Jackson ImmunoResearch Cat #109-136-088, Goat Anti-Human Kappa Light Chain HRP Bethyl Cat #A80-115P, Goat Anti-Mouse Lambda Light Chain HRP Bethyl Cat #A90-121P, Goat Anti-Mouse IgG-APC Jackson ImmunoResearch Cat #115-136-146, Anti-His MAb, (clone AD1.1.10) R&D Systems Cat #MAB050, Anti-His-HRP (clone AD1.1.10) R&D Systems Cat #MAB050H.

Commercially antibodies used for in vivo experiments: Anti-mouse CD8 (clone 53.6.72) BioXCell Cat #BP0004-1, Anti-mouse CD4 (clone GK1.5) BioXCell Cat #BE0003-1, Anti-mouse PD1 (clone 29F.1A12) BioXCell Cat #BE0273, Anti-trinitrophenol rIgG2a isotype control (clone 2A3) BioXCell Cat #BE0089, Anti-mouse CTLA-4 (clone 9H10) BioXCell Cat #BE0131, Anti-mouse PD1 (clone RMP1-14) BioXCell Cat #BE0146.

In-house generated anti-mouse and anti-human antibodies isolated from the n-CoDeR phage display library. Anti-mouse CTLA-4 (clone 5-B07) and Anti-human CTLA-4 (clone 4-E03) are described herein.

### RNA Sequencing

Experimental procedure. CT26 tumor cells were implanted in 10 BALB/c mice per group. Approximately 1 week after implantation when the tumor volume reached 20-50 mm³ (defined as day 0) the mice were non-treated or treated twice, at D0 and D2, by 10⁷ pfu in 50 μL i.t. with either unarmed vaccinia virus (VV empty) or VV_{GM}-αCTLA4. At D4 the tumors were harvested and RNA extracted using the Qiagen kitRNeasy Plus Mini Kit. The samples were conserved at −80° C. till the day of assessment of their quality subsequently. The quality of the purified RNAs was evaluated using Agilent RNA 6000 Nano Kit, Agilent 2100 Bioanalyzer System, 2100 Expert Software to ensure that at least 25% of the RNA fragments were longer than 200 nt (DV200>25%) as required for subsequent 3′ mRNA sequencing. Strand specific libraries were prepared and both ends were sequenced (paired-end sequencing) by IntegraGen, (France) yielding pairs of 100 nt long reads.

Data analysis. Paired reads were processed by a custom bioinformatic pipeline. Briefly, unique molecular identifier (UMI) sequence was extracted from read 1 while the sequence of the 3′ end of the RNA fragment captured was extracted from read 2. After quality-based trimming and quality control, read 2 were mapped with STAR (Dobin et al., 2013) against a custom genome containing Mus musculus full genome (mm10 assembly) plus VV_{GM}-αCTLA4 genome as an artificial extra-chromosome. Reads were then deduplicated using the program dedup from the suit of tools UMI tools (Smith et al., 2017) with the method “unique”. Finally, deduplicated reads were quantified using HTSeq-count (Anders et al., 2015). Readcount data per sample was then normalized using DESeq2 (Love et al., 2014) and a gene was considered differentially expressed if the fold change between two conditions was above 2, and the adjusted p-value below 0.1 (Benjamini-Hochberg correction for multiple testing). Gene Ontology (GO) enrichment analyses were performed using the set of genes differentially expressed as defined above, either up or down-regulated, with the function enrichGO from the R package clusterProfiler (Yu et al., 2012).

### Isolation of Treg Depleting Antibodies Specific for Tumor Treg-Associated Receptors

Antibodies specific for tumor Treg cell-associated receptors were isolated by subjecting the in vitro CDR shuffled n-CoDeR® antibody library to differential biopanning of tumor-associated Treg cells (isolated from CT26, 4T1, B16 and Lewis lung tumor-bearing mice) versus CD4⁺ T cell-depleted naïve cells and CD11b⁺ cells from tumor-bearing mice essentially as described previously (Veitonmaki et al., 2013).

### CTLA-4 mAb Generation

Antibody fragments against human/mouse CTLA-4 were isolated from the n-CoDeR® scFv phage display library. Enrichment of specific CTLA-4 antibodies was achieved by three consecutive pannings using biotinylated h/mCTLA-4-His protein (Sino Biological) loaded on Streptavidin Dynabeads or polystyrene balls. The third selection round also included suspension adapted HEK293-EBNA cells transient transfected with cDNA (Sino Biological) encoding the extracellular and transmembrane regions of h/mCTLA-4 or an irrelevant non-target protein. Pre-selection occurred prior to each selection with a biotinylated non-target protein. Binding phages were eluted after each selection round by trypsin digestion and amplified on plates using standard procedures. Phagemids from selection 3 were converted to scFv producing format and used in subsequent screening assays where specific binding to soluble (recombinant protein) and cell bound antigens (transient transfected cells) was assessed. Commercial antibodies were used for the evaluation of recombinant and cell surface bound human (Yervoy, Bristol Myers Squibb; anti-human APC, Jackson) and anti-mouse CTLA-4 (BioLegend) CTLA-4 by flow cytometry, fluorescence microarray technology (FMAT) and ELISA. Corresponding isotype controls were included as negative controls in all experiments. For primary screening of scFv, h/mCTLA-4 transfected cells were seeded into FMAT plates. E. coli expressed scFv were added followed by deglycosylated mouse anti-His antibody (R&D Systems) and anti-mouse-APC (Jackson). Stained cells were detected using the 8200 detection system (Applied Biosystems). Positive clones from the primary screening were re-expressed and re-tested for binding to transfected cells and to recombinant protein in ELISA. For ELISA, E. coli expressed scFv were added to plates coated with h/mCTLA-4 or non-target protein. Bound scFv were detected using anti-FLAG-AP (Sigma Aldrich) followed by substrate addition (CDP-star, Life Technologies) and luminescence reading (Tecan Ultra).

In total 42 and 31 unique clones were converted to hIgG1 and mIgG2a variants, respectively. VH and VL were PCR amplified and inserted into expression vectors containing the heavy- and light-chain constant regions of the antibody, respectively, and transfected into suspension adapted HEK 293EBNA cells (ATCC). Culture media was harvested 6 days post-transfection and antibodies were purified using columns packed with MabSelect (GE Healthcare) connected to an ÄKTA Purifier system, according to standard procedures. Antibodies were eluted with a low-pH buffer and then dialyzed to an appropriate formulation buffer using a Spectra/Por Dialysis Membrane 4 (Spectrum Laboratories Inc) before a final sterile-filtration.

Antibody purity was assessed by CE-SDS (LabChip XII; Perkin Elmer, Massachusetts, USA) and SE-HPLC (Ultimate 3000, Thermo Fisher Scientific). All preparations were endotoxin low (<0.1 EU/mg protein) as determined using the Chromogenic LAL-Endochrome-K kit (Charles River) adapted to European Pharmacopoeia 2.6.14, current version: Bacterial Endotoxins, “Method D. Chromogenic Kinetic method”.

Purified IgG was then assessed for binding to transfected HEK cells as well as primary cells and to recombinant protein, in both ELISA and Biacore.

### Surface Plasmon Resonance

Binding to recombinant protein was also tested with the surface plasmon resonance (SPR) technology, using Biacore 3000. Anti-human Fc (GE Healthcare) was immobilized on a CM5 sensor chip (GE Healthcare) as a capture antibody with a concentration of 330 nM. Optimal concentrations of 4-E03 and ipilimumab together with the recombinant protein were assessed in pre-tests to obtain good curve fitting and limit mass transfer. Antibodies (5 nM in this particular experiment) were added at 10 μl/min for 1 min, followed by titrating concentrations (1.6 to 50 nM for 4-E03 and 1.6 to 200 nM for ipilimumab) of human CTLA-4 protein (Sino Biological) at 30 μl/min, for 3 min. The surface was regenerated with 10 mM glycine, pH 1.5, between each cycle.

### Cell Transfections

CDNA encoding human and mouse (Sino Biological) CTLA-4 was transfected into suspension adapted 293 FT cells (Life Technologies) using Lipofectamine 2000 (Life Technologies). The transfected cells were cultured in FreeStyle™ 293 Expression Medium (Life Technologies) at 37° C. and 5% CO2, 120 rpm for 48 h. Target expression was analysed using flow cytometry.

### Data Availability

The assigned accession number for RNAseq data reported in this paper is GEO: GSE176052.

### Quantification and Statistical Analysis

All statistical analyses were carried out using GraphPad Prism 9.0 (GraphPad Software Inc, La Jolla, CA). p values were calculated using Students t-tests or One-way ANOVA. The survival periods to the humane end point were plotted using the Kaplan-Meier method with analysis for significance by the log-rank test. Significance was accepted when p<0.05.

### Results

Identification and characterisation of Treg depleting anti-CTLA-4 antibodies Immune checkpoint blockade and anti-CTLA-4 antibody therapy are clinically validated approaches, yet mechanisms underlying anti-CTLA-4 antibody efficacy are incompletely characterised. The central role of CTLA-4 in maintaining tolerance to autoantigen, whilst allowing effective T cell-mediated recognition and removal of foreign antigen expressing cells and tumor cells, is well established (Leach et al., 1996; Tivol et al., 1995; Waterhouse et al., 1995). Accumulating data suggests that anti-CTLA-4 antibodies, besides acting to lower the threshold for T cells to recognize tumor antigen and reject tumors, may exert therapeutic activity through depletion of intratumoral Treg cells following antibody interactions with FcγR-expressing effector cells (Peggs et al., 2009; Simpson et al., 2013) (Ingram et al., 2018). Consistent with this, recent data indicated a role for FcγRs and Treg depletion in efficacy of anti-CTLA-4 antibodies including ipilimumab (Arce Vargas et al., 2018).

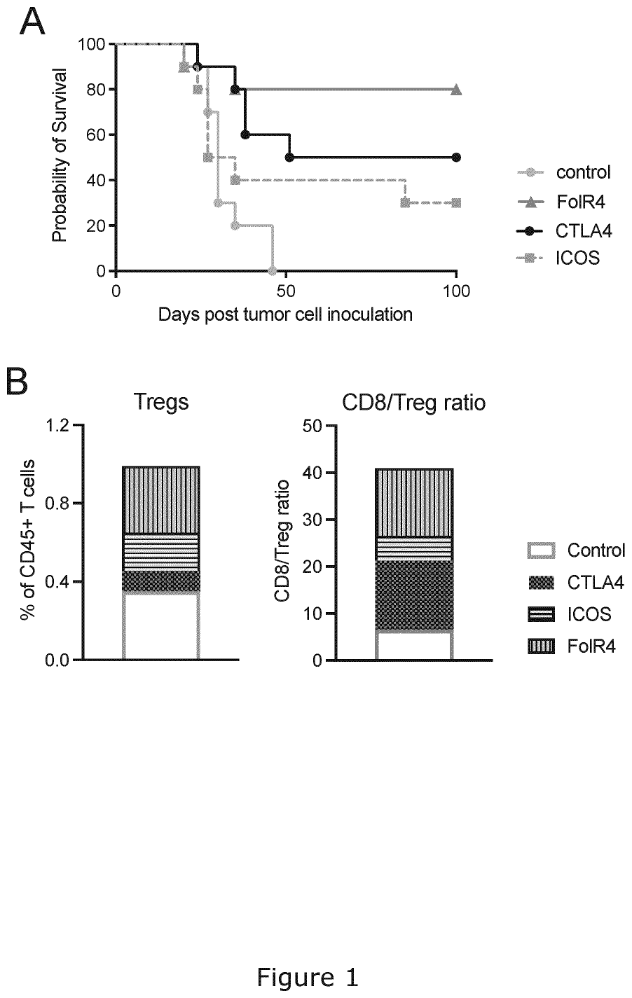

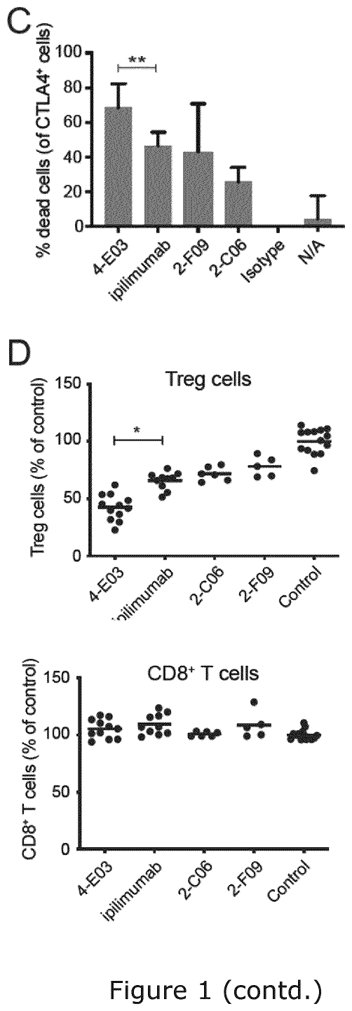

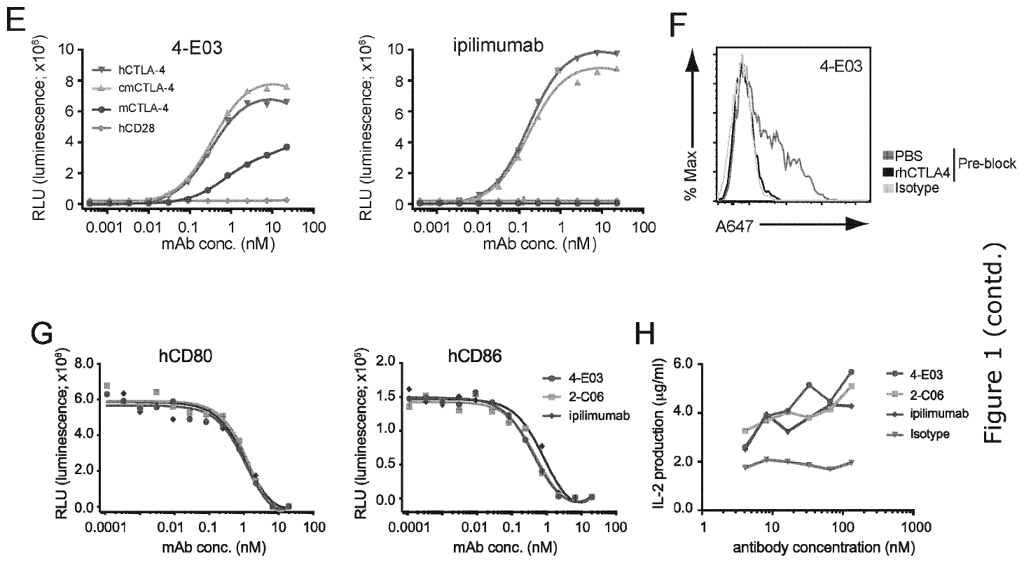

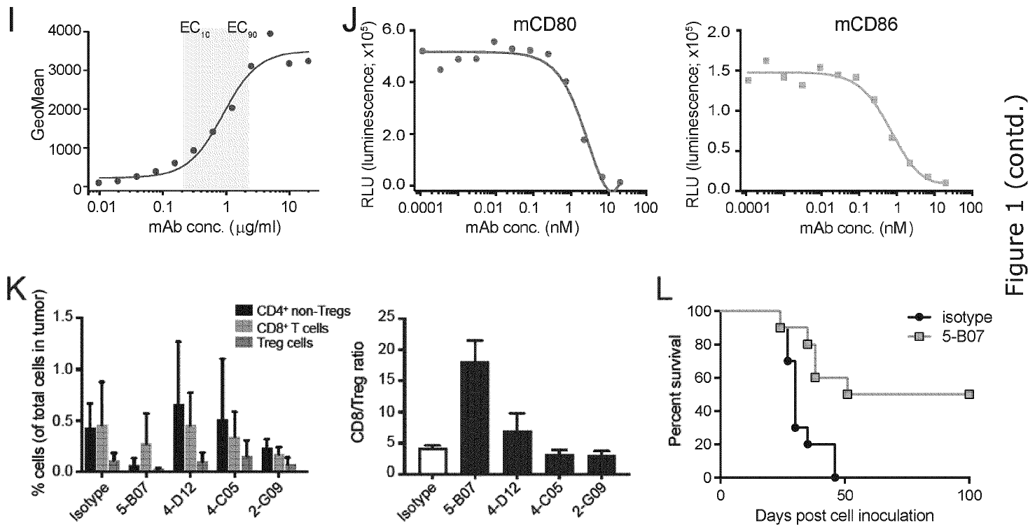

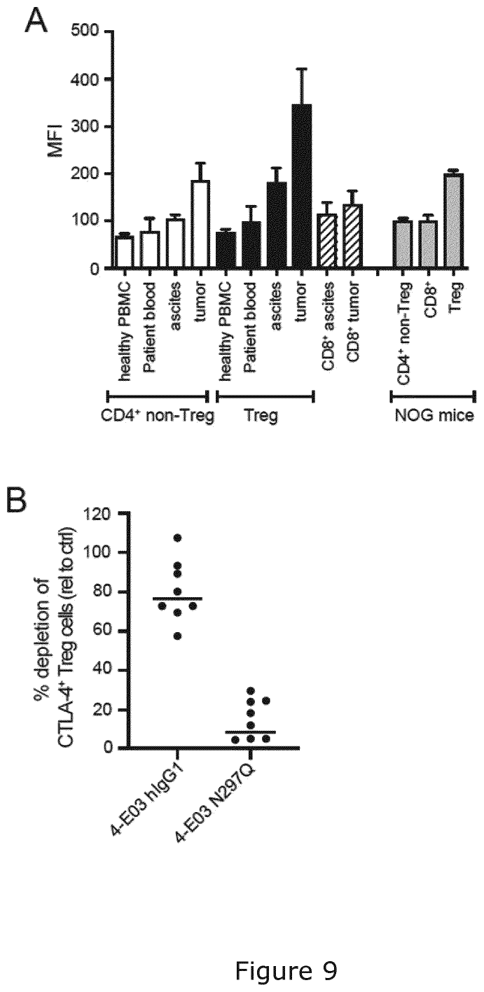

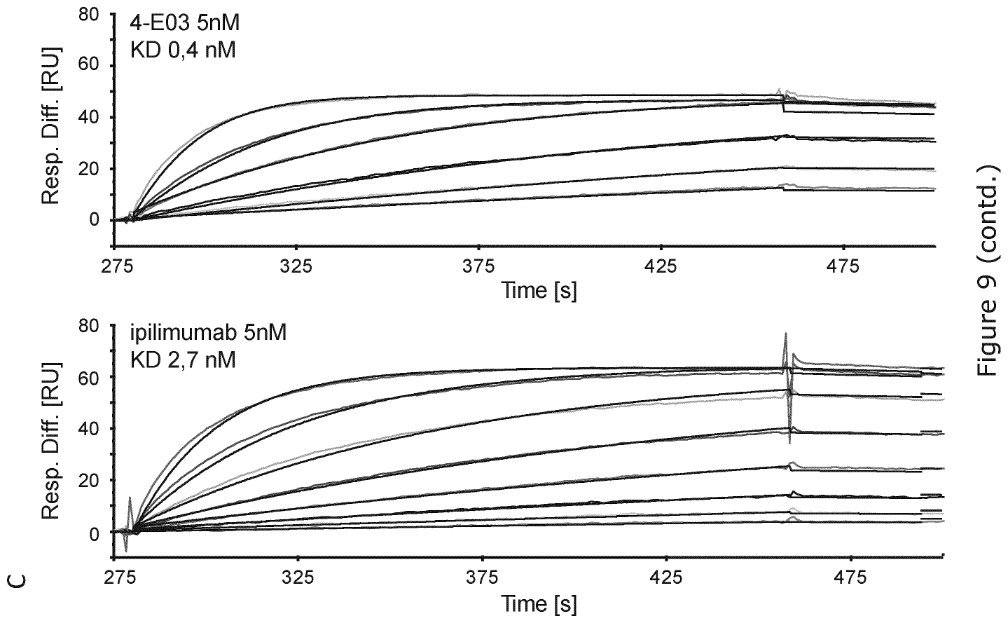

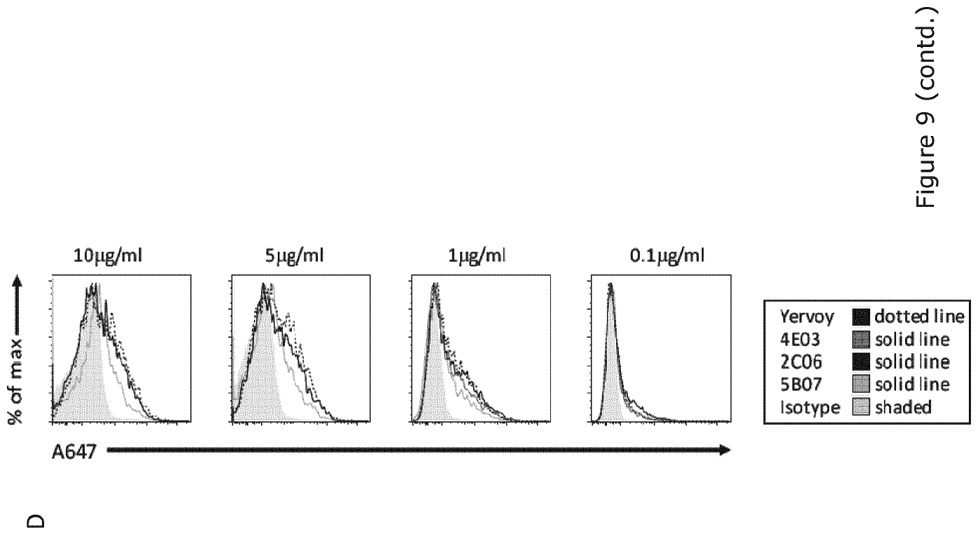

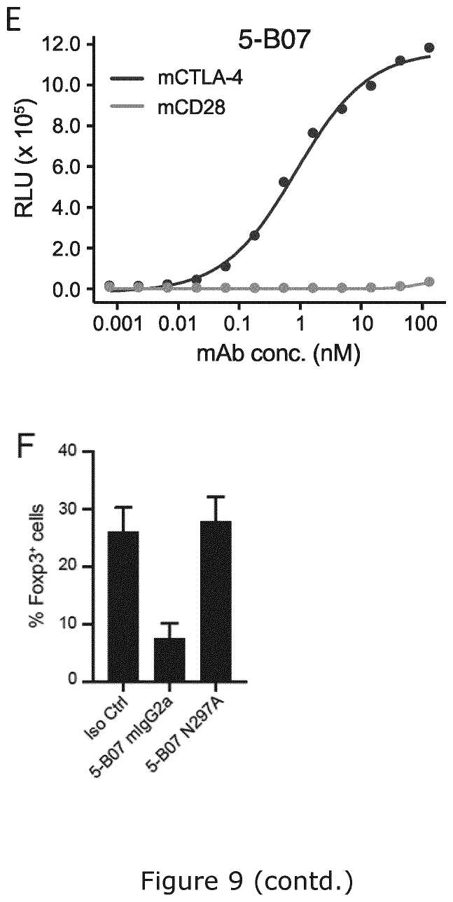

Using the target agnostic F.I.R.S.T™ discovery platform (Veitonmaki et al., 2013), the inventors identified an array of antibodies and their associated targets capable of depleting Treg cells and improving survival in the T cell inflamed CT26 mouse tumor model (data not shown, FIGS. 1A and B). CTLA-4 and anti-CTLA-4 antibodies were identified amongst these, and anti-CTLA-4 mIgG2a mAb depleted intratumoral Treg cells and conferred survival upon treatment of animals bearing syngeneic CT26 tumors (FIG. 1A). Focused screening of human CTLA-4-specific IgG1 antibodies identified several clones with similar in vitro depleting activity of CTLA-4 expressing human T cells (FIG. 1C). One clone (4-E03) stood out based on its consistently stronger CTLA-4⁺ T cell depleting efficacy compared to other clones when screened across multiple donors (FIG. 1C). To investigate the in vivo relevance of this clone's apparent stronger depleting activity, the inventors turned to a model where human PBMCs are engrafted into NOD SCID IL-2R gamma−/− (NSG) mice. Owing to a graft-versus host type of interaction human Treg and CD8⁺ T cells are strongly activated, and show similar co-stimulatory and co-inhibitory molecule expression compared to that observed in human tumors (Buchan et al., 2018) (FIG. 9A). Analyses of NOG-hPBMC CD4⁺ CD25⁺ CD127^{low} Treg and CD8⁺ T cells indeed revealed similar CTLA-4 expression levels to that observed on T cells obtained from 9 ovarian cancer patients (FIG. 9A). Furthermore, dosing of NOG-hPBMC mice with 10 mg/kg ipilimumab or additional anti-CTLA-4 antibody clones (2-C06 and 2-F09) demonstrated similar in vivo depleting activity of human CD4⁺ CD25⁺ CD127^{low} Treg cells (FIG. 1D). However, 4-E03 evoked significantly greater depletion of human Treg cells. Importantly, and consistent with the lower expression of CTLA-4 on intratumoral and NOG-hPBMC CD8⁺ T cells compared to Treg cells (FIG. 9A), 4-E03 showed no depletion of human activated CD8⁺ T cells (FIG. 1D). Biochemical characterization, in particular HPLC-SEC analyses of 4-E03 antibody preparations showed >95% monomeric IgG (data not shown), ruling out that 4-E03-enhanced Treg depletion resulted from aggregation of antibody. Consistent with the inventors and others observations, anti-CTLA-4 mAb Treg depletion was shown to depend on antibody Fc:FcγR interactions. An FcγR-binding impaired variant of 4-E03 (IgG1N297Q) showed severely impaired Treg depletion compared to WT FcγR-proficient IgG1 (FIG. 9B).

These findings indicated that 4-E03 binds to a functionally distinct epitope on CTLA-4. The inventors therefore characterised the binding and ligand-blocking activity of 4-E03 relative to ipilimumab and other anti-CTLA-4 antibodies. All antibodies showed high specificity for the extracellular domain of human CTLA-4, and no observable binding to its closely related human homologue CD28 by ELISA (FIG. 1E). While 4-E03 and ipilimumab bound with similar potency and efficacy to hCTLA-4 (FIG. 1E and FIG. 9C) only 4-E03 showed weak but clear cross-reactivity with mouse CTLA-4 (FIG. 1E). These findings are consistent with the two antibodies binding to distinct epitopes.

Further comparative analyses of 4-E03 and ipilimumab assessing binding to endogenously expressed CTLA-4 on in vitro-activated human CD4⁺ T cells (FIG. 9D and FIG. 1F), blockade of B7:CTLA-4 interaction (FIG. 1G), or inhibition of B7:CTLA-4-mediated T cell suppression (FIG. 1H), revealed otherwise near identical efficacy and potency. In conclusion, the results indicated that 4-E03 binds to a functionally distinct CTLA-4 epitope, which is associated with stronger Treg depletion but equivalent blockade of B7:CTLA-4 induced T effector cell suppression to the epitope targeted by ipilimumab.

Since 4-E03 cross-reacted only weakly with mouse CTLA-4 (FIG. 1E), the inventors next focused on screening to identify an appropriate surrogate for in vivo proof-of-concept studies in immunocompetent mouse tumor models. One clone (5-B07), which showed highly specific binding to mouse CTLA-4 transfected CHO cells (FIG. 1I) and mouse CTLA-4 protein (FIG. 9E), blockade of B7:CTLA-4 interactions (FIG. 1J) and similar strong depletion of intratumoral mouse Treg (FIG. 1K) compared with 4-E03 in the human setting, was identified. Further, anti-mCTLA-4 (5-B07) conferred antitumor activity and improved survival of CT26-tumor bearing BALB/c mice (FIG. 1L). As observed with anti-hCTLA-4 (4-E03) on human cells, anti-mCTLA-4 (5-B07) depletion of mouse Treg was found to depend on Fc:FcγR interactions. Fc:FcγR-binding proficient but not Fc:FcγR-binding impaired variants of 5-B07 depleted intratumoral Tregs (FIG. 9F).

The similar pronounced Treg-depleting activity, alongside their similar high specificity for CTLA-4 and blocking activity of CTLA-4:B7-family interactions, indicated the therapeutic potential of anti-hCTLA-4 (4-E03) and of anti-mCTLA-4 (5-B07) as a suitable MoA-matched surrogate.

### Engineering of Antibody-Encoding Oncolytic Viruses for Tumor-Selective CTLA-4 Blockade and Treg Depletion

The frequent side effects of anti-CTLA-4 antibody therapy are consistent with the well-established role of CTLA-4 acting as a central checkpoint to maintain T cell homeostasis and tolerance to self (Tivol et al., 1995; Waterhouse et al., 1995). Recent work by Quezada and colleagues has however indicated that intratumoral Treg depletion may significantly contribute to ipilimumab clinical activity (Arce Vargas et al., 2018), and intratumorally delivered Treg depleting antibodies may afford substantial antitumor activity in mouse tumor models (Fransen et al., 2013; Marabelle et al., 2013b). This dual activity of anti-CTLA-4, acting in central and peripheral compartments respectively, suggests that localising anti-CTLA-4 therapy to tumors may be an attractive strategy to uncouple anti-CTLA-4 efficacy from toxicity.

The inventors hypothesised that intratumorally delivered oncolytic viruses (OVs) engineered to express Treg depleting anti-CTLA-4 would represent a particularly attractive means to achieve effective, yet safe, tumor-localised anti-CTLA-4 therapy. Besides enabling local antibody production and blockade of CTLA-4 receptors and Treg depletion in the TME upon infection of tumor cells, OVs are thought to exert both direct and indirect antitumor activity and have been approved for cancer immunotherapy (Bommareddy et al., 2018).

The inventors therefore engineered a Vaccinia virus vector, derived from an attenuated Copenhagen strain (Foloppe et al., 2019) with clinically proven safety and strong immunomodulatory effects observed in global Smallpox vaccination programs, and cytolytic and inflammatory cell infiltration-inducing properties in mouse experimental models of immune desert and immune excluded cancer (Fend et al., 2017; Kleinpeter et al., 2016; Liu et al., 2017; Marchand et al., 2018), with full-length anti-hCTLA-4 or anti-mCTLA-4 IgG antibody sequences. Variant vectors additionally encoding GM-CSF (VV_{GM}-αCTLA4) (FIG. 2A), a growth factor inducer and enhancer of myelopoiesis and innate immune cell chemotaxis, were also generated and evaluated for therapeutic efficacy.

Following genetic reconstruction, recombinant anti-CTLA-4 encoding viruses were confirmed to infect, replicate in (FIG. 2B), and lyse (FIG. 2C) tumor cell lines. Tumor cell lines infected with engineered oncolytic viruses were further shown to produce full-length IgG antibody and GM-CSF transgenes (FIGS. 2D and 2E) with equipotent binding to CTLA-4 receptors (FIG. 2G) and support of GM-CSF-dependent TF-1 cell proliferation (FIG. 2F) compared to recombinantly produced proteins. 4-E03 produced by BT-001-infected MIA-PaCa-2 tumor cells was also shown to deplete human Treg cells in vivo (FIG. 2H).

Intratumoral VV_{GM}-αCTLA4 has Antitumor Activity Associated with Tumor-Selective CTLA-4 Receptor Saturation and Treg Depletion

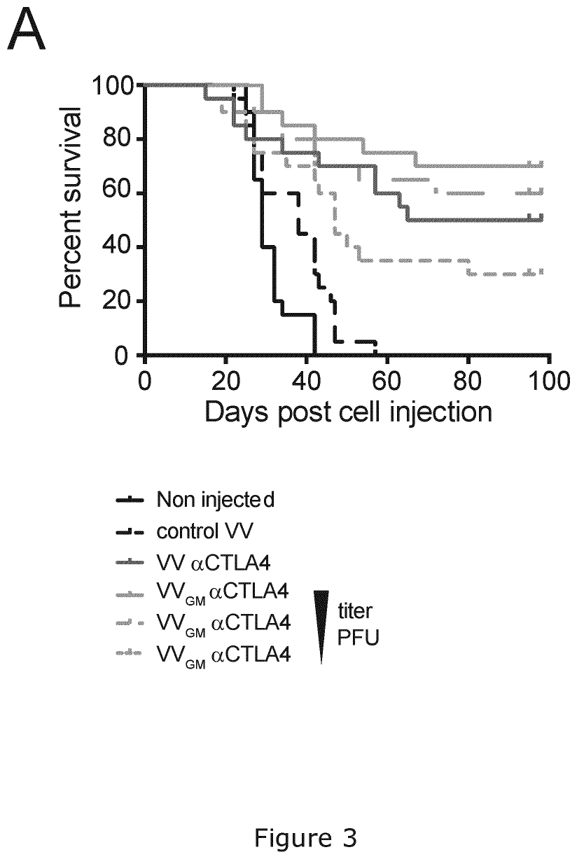

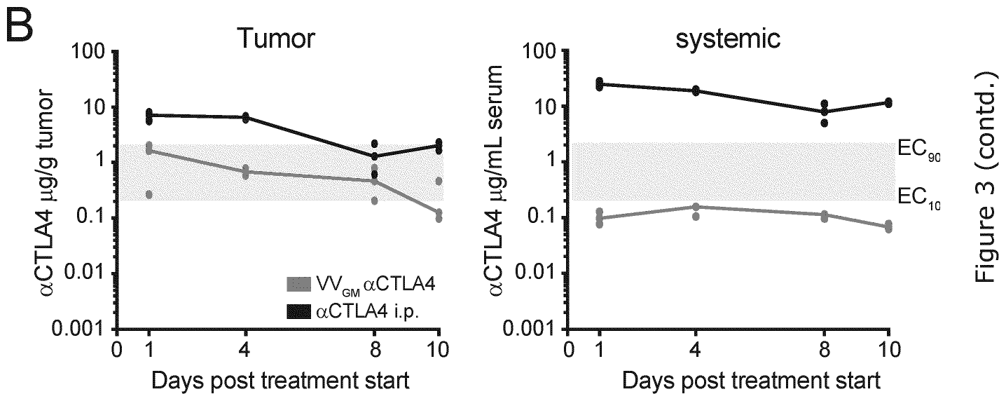

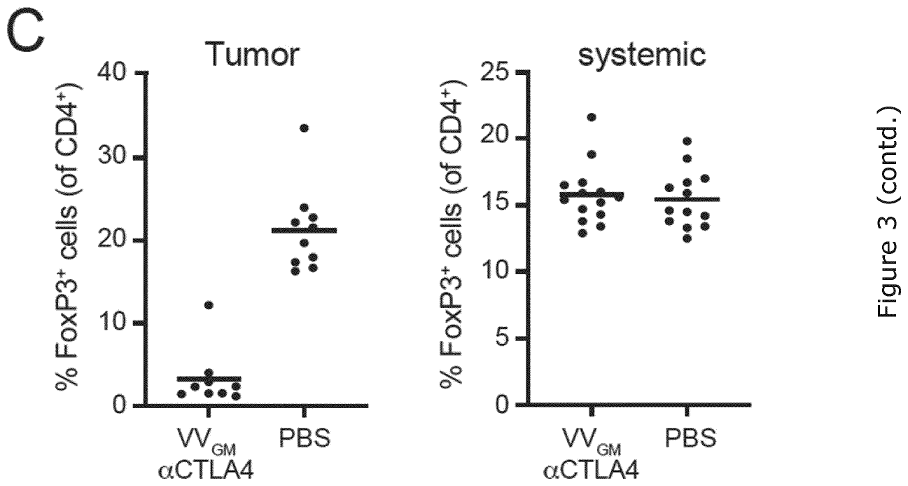

VV_{GM}-αCTLA4 antitumor activity was first assessed in the CT26 BALB/c model known to be highly infiltrated by T cells and sensitive to systemic anti-CTLA-4 antibody treatment (Grosso and Jure-Kunkel, 2013). Three intratumoral injections with 7.5×10⁴, 7.5×10⁵ or 7.5×10⁶ pfu of VV_{GM}-αCTLA4 to CT26 tumor-bearing animals demonstrated a dose-dependent antitumor effect, which peaked at 10⁶-10⁷ pfu, with 6-7/10 animals cured (FIG. 3A). Treatment with control virus lacking anti-CTLA-4 antibody and/or GM-CSF transgenes demonstrated an obligate dependence on anti-CTLA-4 antibody (0/10 mice surviving), and a marginal anti-CTLA-4 enhancing effect of GM-CSF, for therapeutic efficacy (7/10 versus 5/10 mice surviving). Based on the established dose-dependent, anti-CTLA-4-antibody-dependent, and GM-CSF enhancing effects the inventors therefore investigated therapeutic and mechanistic evaluation and characterisation on the double transgene-encoding OV (VV_{GM}-αCTLA4) using an intratumorally delivered dose of 1×10⁷ pfu.

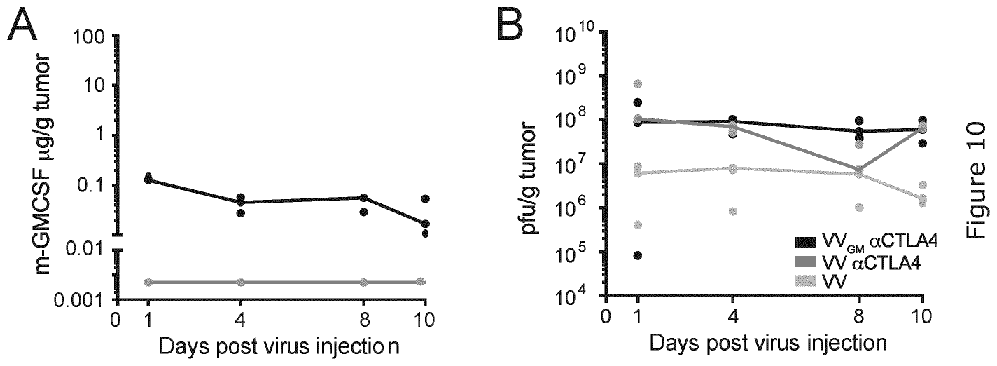

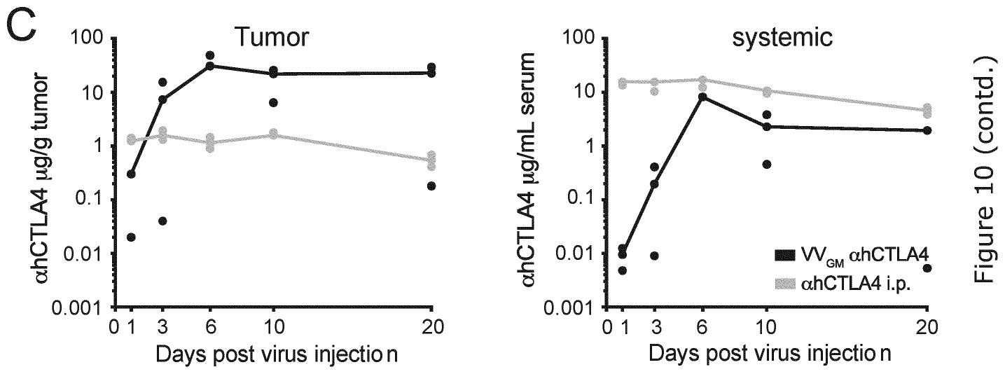

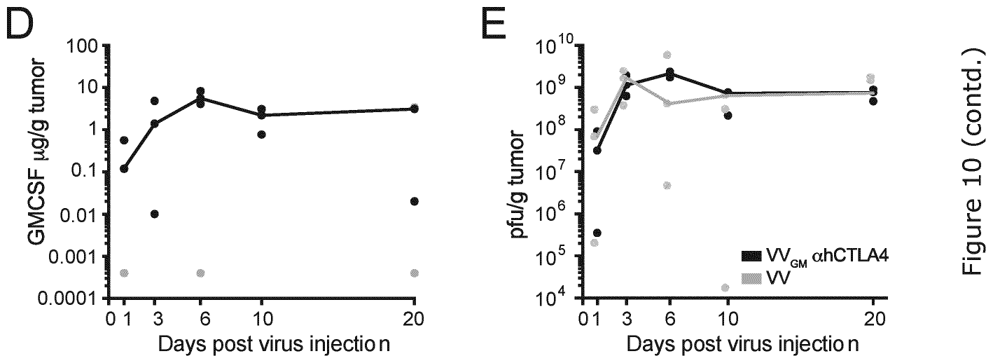

The inventors next assessed tumor and systemic concentrations of anti-CTLA-4 (FIG. 3B), GM-CSF (FIG. 10A), and viral particles (FIG. 10B) following intratumoral administration of the transgene-encoding Vaccinia OV. Intratumoral injection of 10⁷ VV_{GM}-αCTLA4 infectious particles into syngeneic mouse tumor-bearing immune competent mice generated intratumoral antibody exposure associated with sustained saturation of tumor, but not blood, CTLA-4 expressing cells (FIG. 3B and FIG. 1I). Similarly, i.t. administration of VV_{GM}-αhCTLA4 to human tumor xenograft-bearing immune deficient mice generated orders of magnitude greater antibody concentrations in tumor compared with blood (FIG. 10C-E). Consistent with intratumoral administration achieving receptor saturating concentrations in tumor but not systemic compartments (FIG. 3B), i.t. VV_{GM}-αCTLA4 resulted in near complete depletion of intratumoral Treg cells but did not affect Treg numbers in spleens of CT26 tumor-bearing BALB/c mice (FIG. 3C).

Collectively the results demonstrated that intratumoral administration of anti-CTLA-4-encoding Vaccinia virus successfully achieved tumor-restricted CTLA-4 receptor saturation and Treg depletion in vivo, supporting its tumor-selective anti-CTLA-4 therapeutic nature and prompting testing of in vivo efficacy and tolerability in diverse cancer experimental models.

### VV_{GM}-αCTLA4 has Broad Antitumor Activity

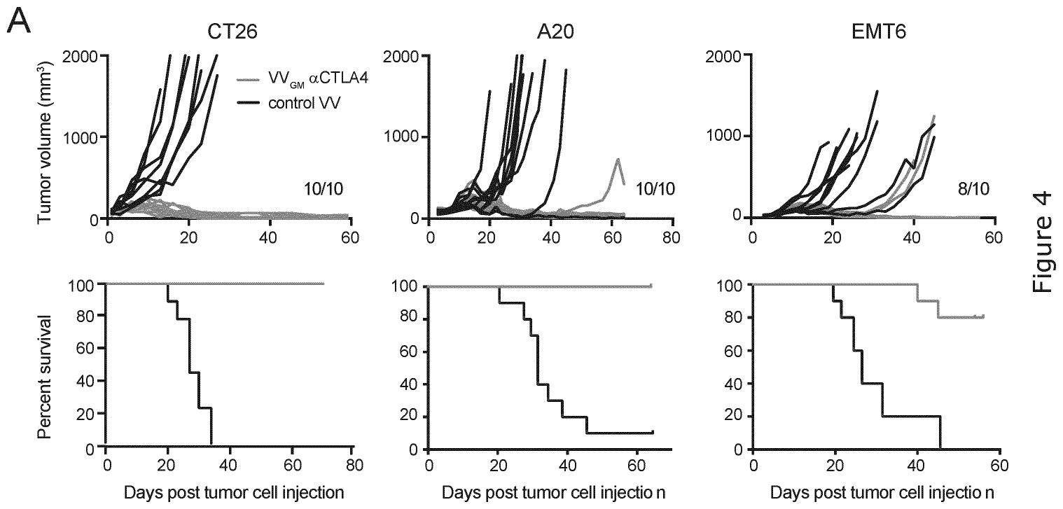

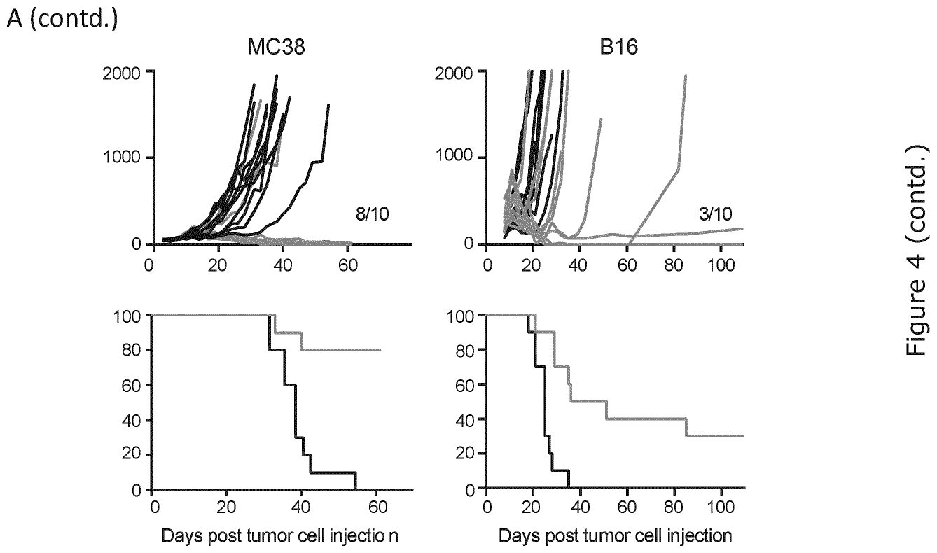

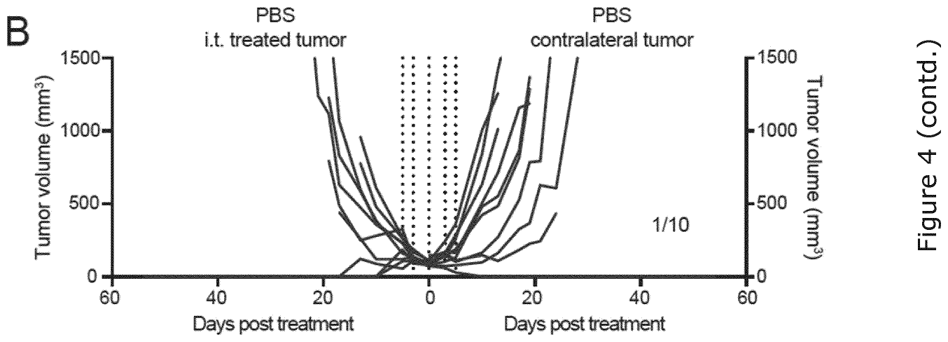

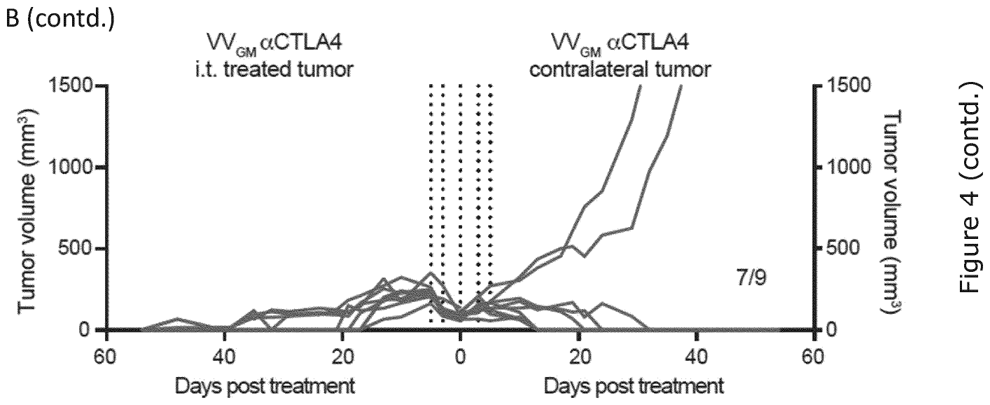

The inventors proceeded to assess the anti-tumor activity of i.t. VV_{GM}-αCTLA4 in a range of immune competent mouse cancer models spanning hematologic (A20) and solid cancers of different origin on different genetic mouse backgrounds (CT26 BALB/c colon; EMT6 BALB/c breast, MC38 C57BL/6 colon and B16 C57BL/6 melanoma, FIG. 4A), representing highly T cell inflamed (CT26) to immune excluded (B16) tumor microenvironments. The models included those sensitive or resistant to ICB with anti-CTLA-4 or anti-PD-1.

Strikingly, i.t. administration of VV_{GM}-αCTLA4 to C57BL/6 or BALB/c mice carrying established syngeneic tumors characterized by diverse immune inflamed types of tumor microenvironment cured the majority (A20=10/10, EMT6=8/10, MC38=8/10 and CT26=10/10 surviving mice) of animals (FIG. 4A). Equally impressively, in the B16 C57BL/6 model characterized by an immune desert type of TME and resistance to both anti-PD-1 and anti-CTLA-4, i.t. VV_{GM}-αCTLA4 significantly delayed tumor growth and cured 3/10 animals. These results indicated a broad therapeutic potential of VV_{GM}-αCTLA4 in diverse cancer types, including in patients with diverse inflammatory and immune excluded types of TMEs.

Intratumoral Treatment with VV_{GM}-αCTLA4 Induces Long-Lasting Systemic Antitumor Immunity

Preclinical and clinical studies have demonstrated the therapeutic potential of tumor-localised cancer immunotherapy. Intratumoral oncolytic virotherapy, alone (Andtbacka et al., 2015) or combined with ICB (Chesney et al., 2018; Ribas et al., 2017), induces durable responses in melanoma cancer patients. Mechanistically, i.t. oncovirotherapy has been proposed to induce or enhance inflammatory cell infiltration into injected tumors, resulting in increased tumor antigen-presentation, migration to draining lymph nodes and, following priming, CD8⁺ T cell trafficking to distant (non-injected) tumor lesions to exert systemic antitumor “abscopal” effects (Ngwa et al., 2018). In the clinic, such induction of systemic adaptive antitumor memory responses will be critical since cancer patients may present with wide-spread disease characterized by metastasized, non-detectable, or uninjectable tumors.

The inventors used a multi-pronged approach to assess whether i.t. VV_{GM}-αCTLA4 induced abscopal effects and systemic antitumor immunity. Firstly, using a “twin tumor model” where tumor cells are subcutaneously grafted to right and left flanks of each animal but only one tumor is injected with OV and the other is left untreated, abscopal effects can be evaluated and manifested as reduced tumor growth in uninjected tumors.

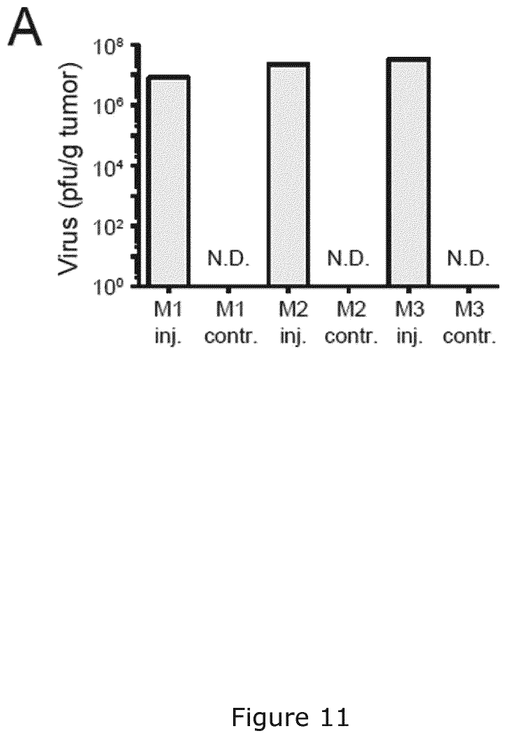

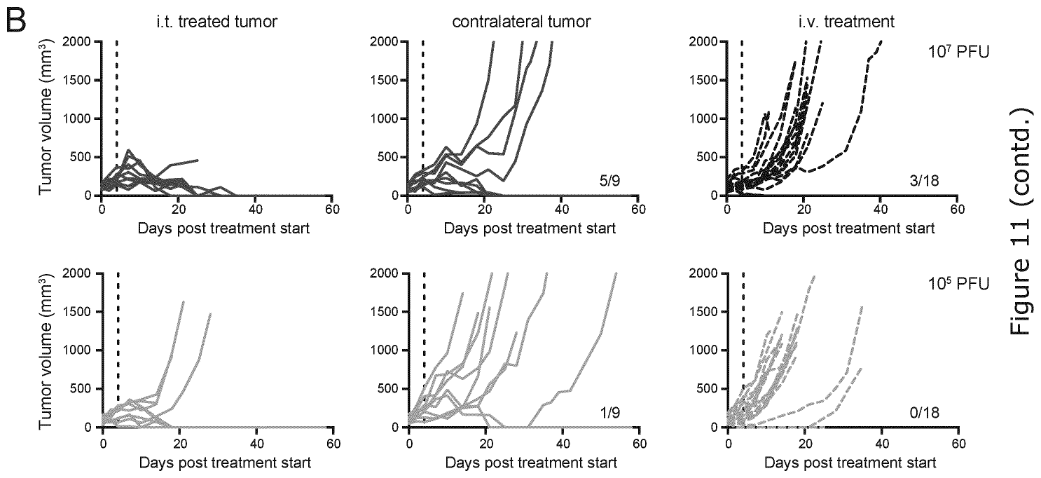

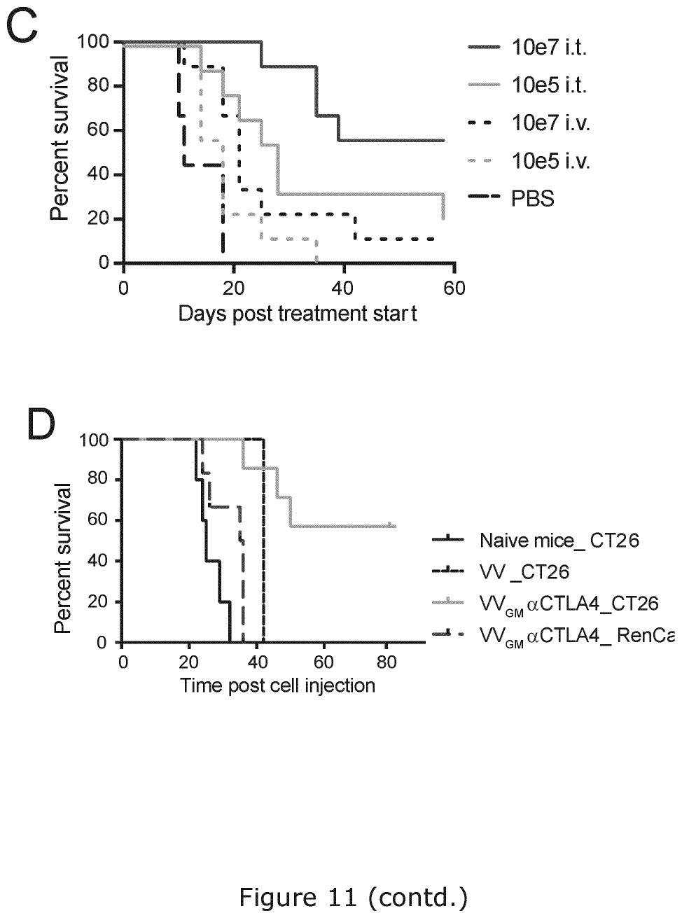

Intratumoral injection of a maximally efficacious VV_{GM}-αCTLA4 dose in CT26 tumor-bearing mice resulted in complete (9/9) rejection of injected tumors and near complete rejection (7/9) of uninjected tumors, indicating a strong abscopal effect (FIG. 4B). The true abscopal nature of i.t. OV administration was confirmed two-fold. Firstly, and to exclude a potential therapeutic effect of viral particle spread from injected to uninjected tumors, non-injected tumors were analysed and confirmed negative for viral particles (FIG. 11A).

Second, using the twin tumor model the inventors compared antitumor activities of therapeutically maximally efficacious (10⁷ pfu) and suboptimal (10⁵ pfu) doses of VV_{GM}-αCTLA4 administered locally (i.t.) or systemically (i.v.). Consistent with a true abscopal effect i.t. administration conferred enhanced survival compared to i.v. administration at both tested doses (FIG. 11C). In fact, i.t. dosing of 10⁵ pfu was at least as efficacious compared with the 100-fold greater i.v. injected dose in rejecting non-injected tumors (FIGS. 11B and 11C). Finally, and consistent with i.t. OV administration inducing immunological memory characteristic of an adaptive antigen-specific immune response, cured animals were protected against re-challenge with the same (CT26), but not unrelated (Renca), tumors (FIG. 11D).

VV_{GM}-αCTLA4 Elicits Robust Systemic CD8⁺ T Cell Dependent Anti-Tumor Immunity

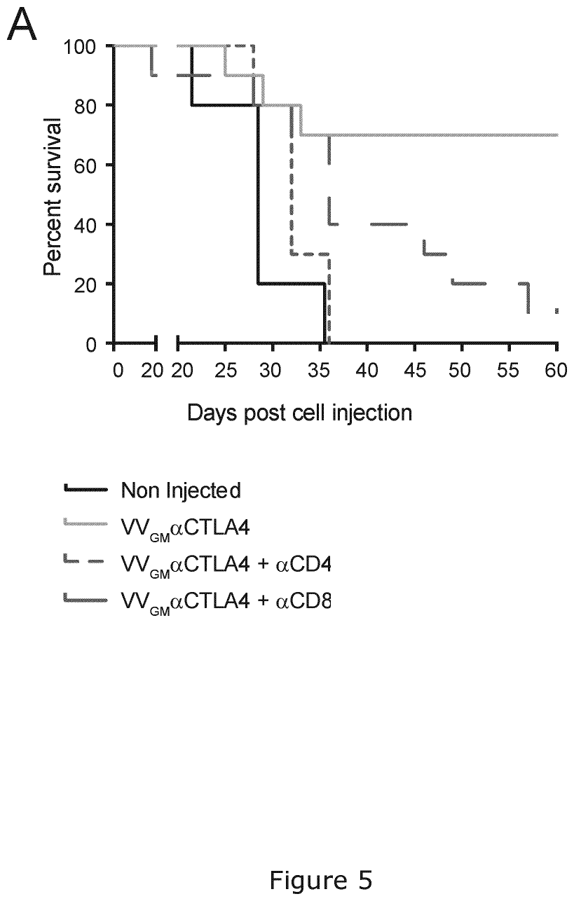

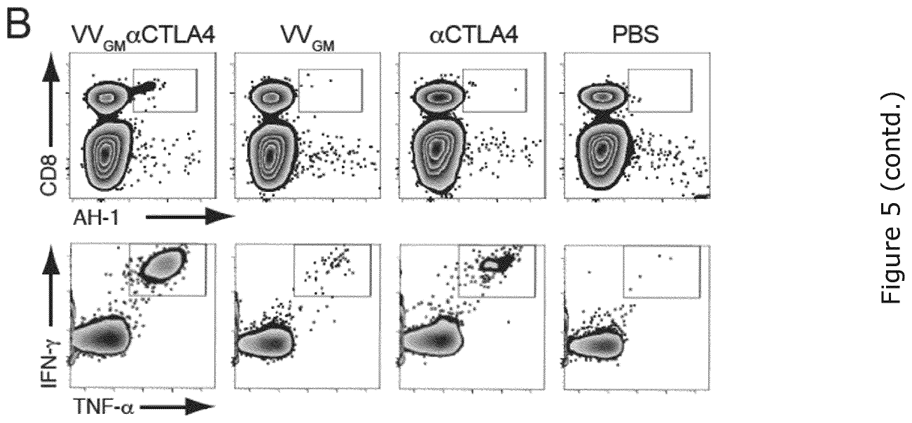

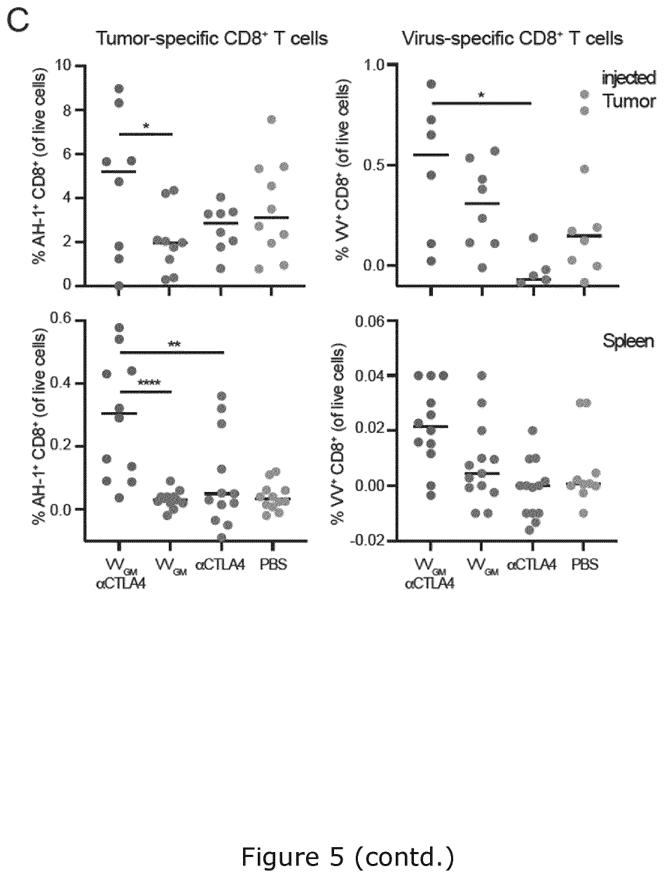

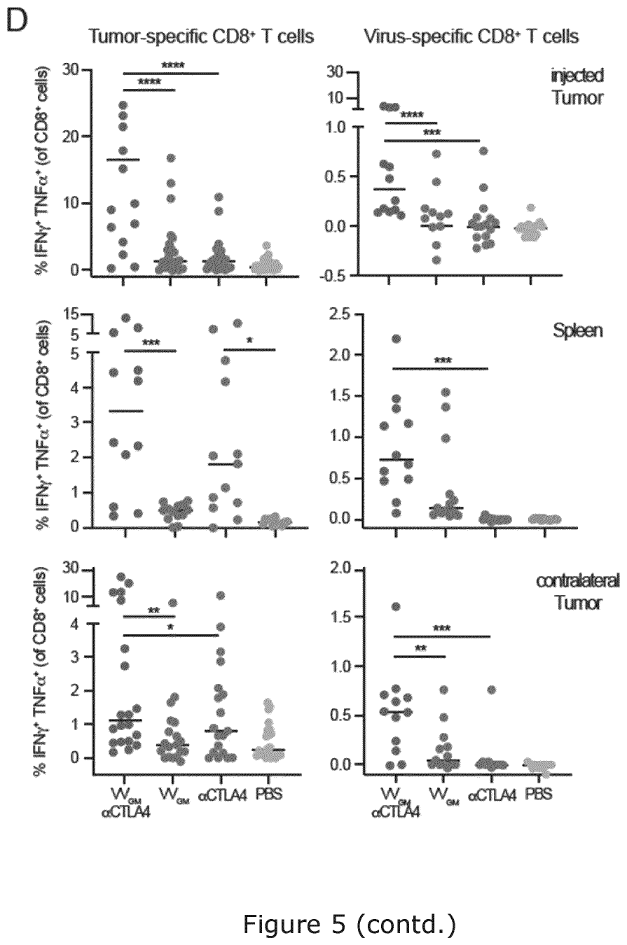

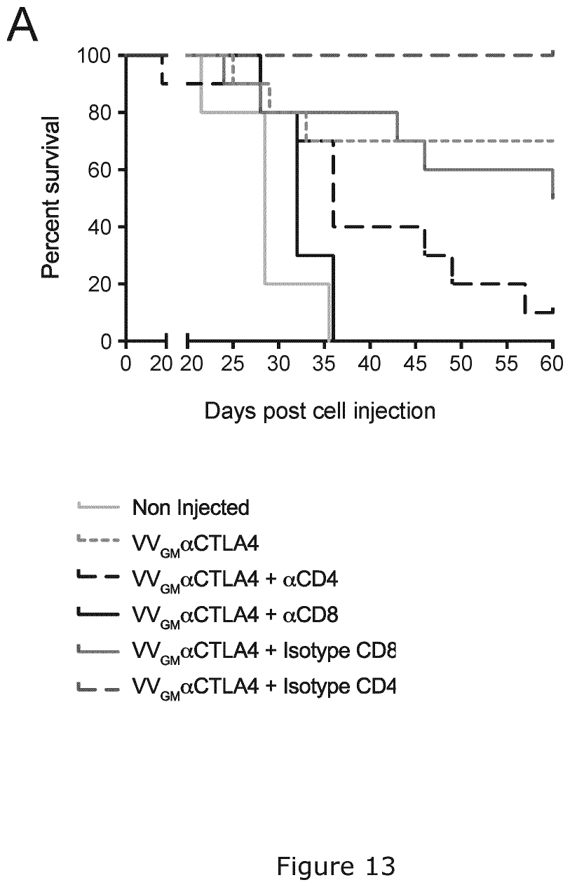

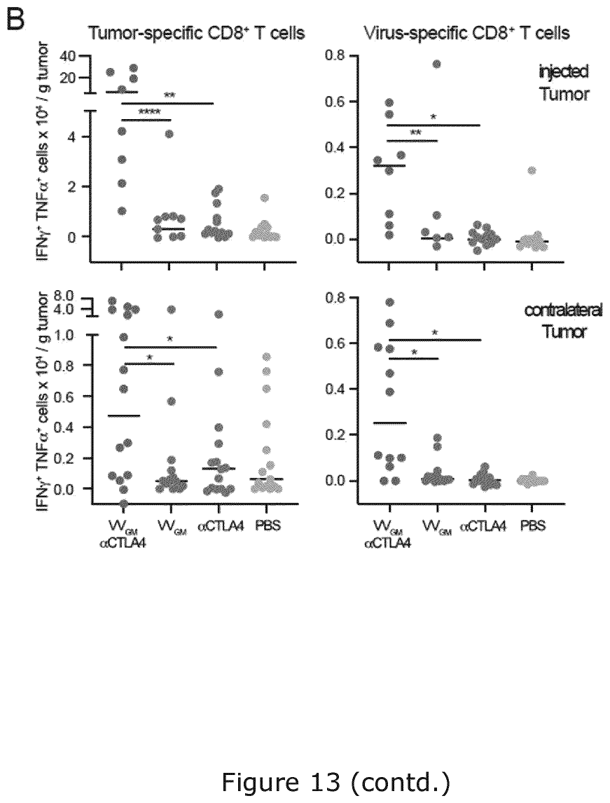

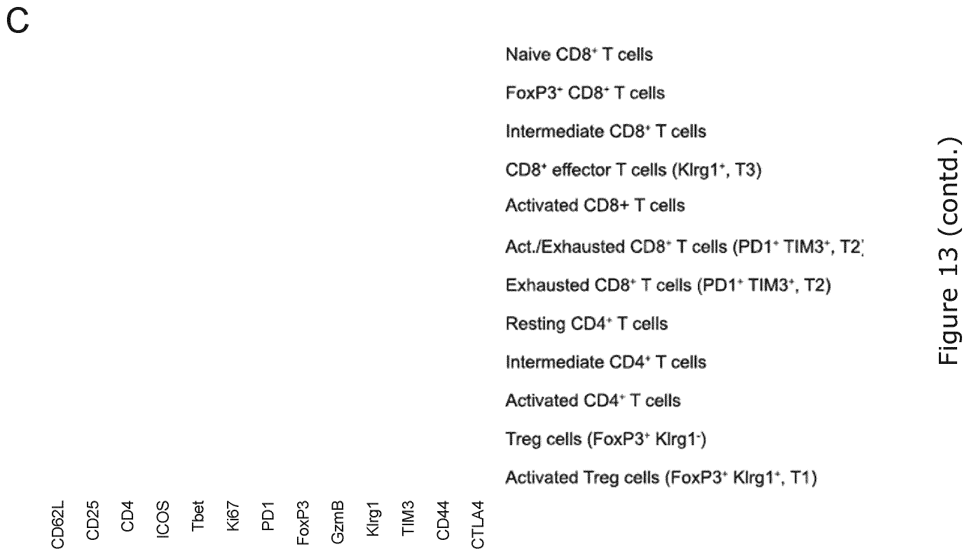

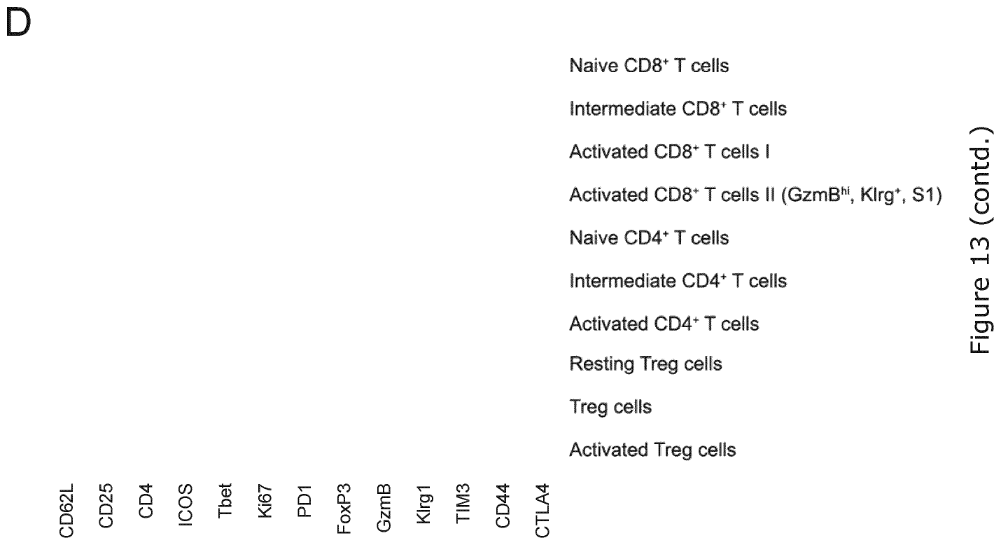

The inventors investigated the nature of the systemic antitumor immune response by assessing VV_{GM}-αCTLA4 therapeutic activity in immune intact compared with CD4⁺ T cell-depleted or CD8⁺ T cell-depleted CT26 tumor-bearing mice (FIG. 5A and FIG. 13A). Strikingly, CD8⁺ T cell depletion completely eliminated VV_{GM}-αCTLA4 antitumor activity. CD4⁺ T cell depletion reduced, but did not ablate, VV_{GM}-αCTLA4 effects. These data demonstrated that VV_{GM}-αCTLA4 antitumor activity critically depends on CD8⁺ T cells. Alongside demonstrated abscopal effects and tumor-specific protection against re-challenge, the results strongly suggested that intratumorally delivered VV_{GM}-αCTLA4 induced robust systemic CD8⁺ T cell antitumor immunity.

The inventors therefore next assessed if i.t. VV_{GM}-αCTLA4 induced or expanded tumor-specific and virus-specific CD8⁺ T cells in tumors and in the periphery. CT26 tumor-bearing BALB/c mice were treated intratumorally with VV_{GM}-αCTLA4 or, to mimic clinically available anti-CTLA-4 regimens, systemically (i.p.) with anti-mCTLA-4 mAb 5-B07 (3 mg/kg). CT26 tumor-specific and Vaccinia-specific CD8⁺ T cells in tumor and central compartments (spleen) were quantified by two approaches; direct quantification of tumor-specific CD8+ T cells in harvested spleens using CT26 tumor antigen (AH-1)-specific and Vaccinia virus-specific multimers and assessment of IFN-γ⁺ TNF-α⁺ CD8⁺ T cells following ex vivo stimulation of splenocytes (FIG. 5B) or of TILs with CT26-derived tumor peptide AH-1 and Vaccinia-derived peptide S9L8, respectively. Treatment with PBS or VV encoding only GM-CSF were included as controls. As expected, and consistent with intratumoral VV_{GM}-αCTLA4 inducing robust systemic CD8⁺ T cell-dependent antitumor immunity, i.t. VV_{GM}-αCTLA4 induced tumor-specific CD8⁺ T cells both in injected tumors and in peripheral (non-injected tumor and spleen) compartments (FIGS. 5B-5D and 13B) as assessed by ex vivo stimulation of splenocytes or dextramer staining. Impressively, i.t. VV_{GM}-αCTLA4 expanded tumor-specific CD8⁺ T cells more effectively compared with systemic anti-CTLA-4. Control treatment with PBS or virus lacking αCTLA-4 did not induce tumor-specific CD8⁺ T cells by either read-out. Interestingly, i.t. treatment with VV_{GM}-αCTLA4 also induced Vaccinia-specific CD8⁺ T cells albeit in low numbers.

Collectively, these data demonstrated that intratumoral VV_{GM}-αCTLA4 induced robust systemic CD8⁺ T cell-dependent antitumor immunity.

Intratumorally Induced CD8⁺ T Cell Antitumor Immunity is FcγR-Dependent and Correlates with Treg Depletion

The broad antitumor activity, strong expansion of tumor-specific CD8⁺ T cells in tumor and periphery, and tumor-restricted depletion of Treg cells, supported a highly effective and safe treatment with i.t. VV_{GM}-αCTLA4.

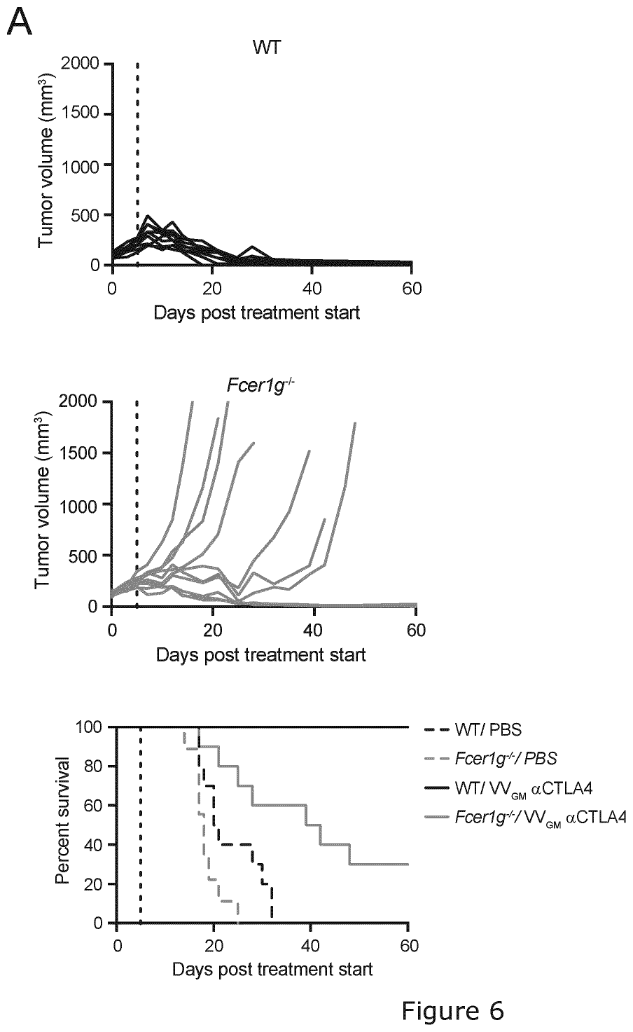

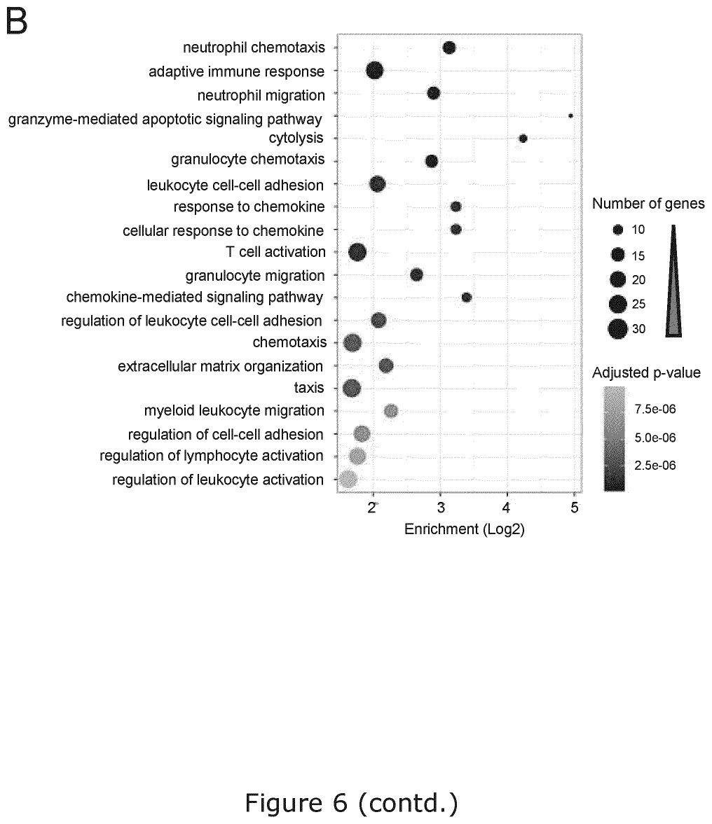

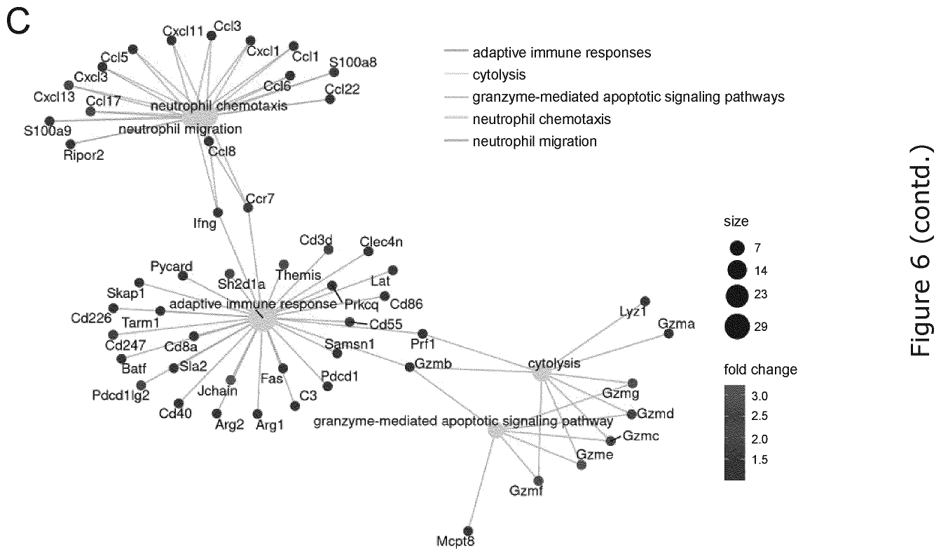

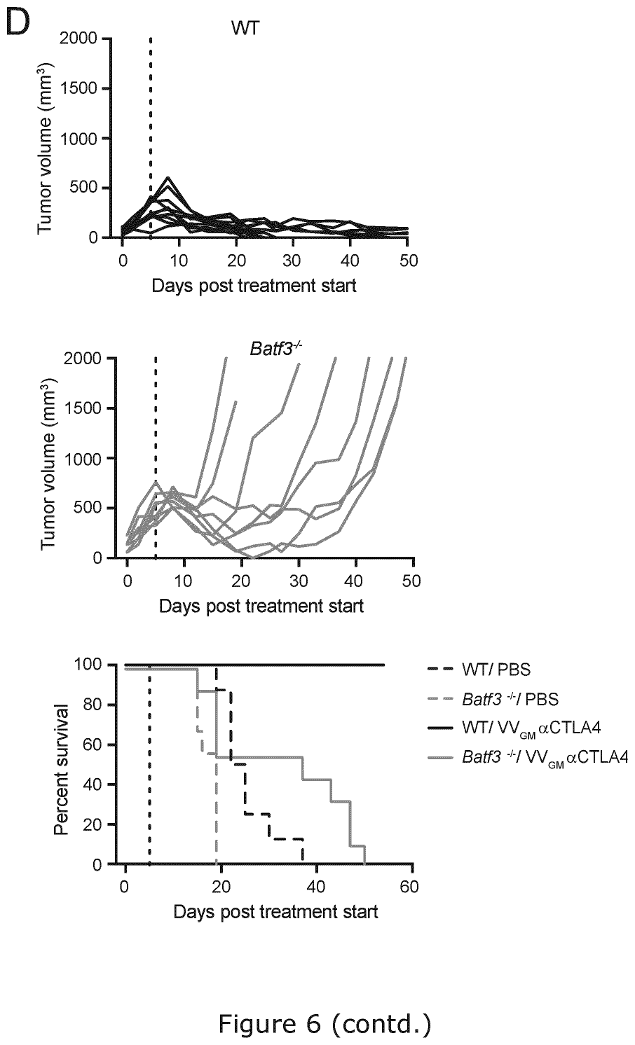

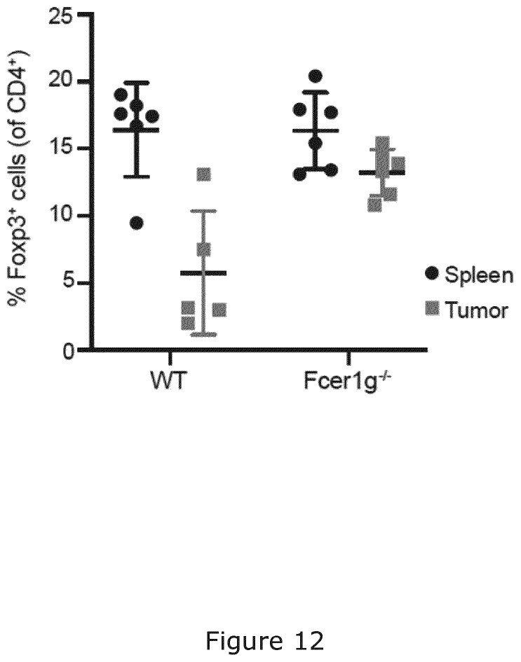

To further assess and confirm a role for antibody-mediated Treg depletion underlying antitumor immunity, the inventors compared antitumor effects of i.t. VV_{GM}-αCTLA4 in CT26 tumor-bearing WT and common gamma chain-deficient (Fcer1g^{-/-}) BALB/c mice. Fcer1g^{-/-} mice lack functional activating Fc gamma receptors and anti-CTLA-4 antibody in vivo Treg depletion and associated antitumor activity was previously shown to be activating FcγR-dependent (Arce Vargas et al., 2018; Simpson et al., 2013). Consistent with anti-CTLA-4-induced Treg depletion critically underlying i.t. VV_{GM}-αCTLA4 antitumor immunity, WT (10/10), but not FcγR-deficient animals (3/10), were completely protected and cured from their cancer (FIG. 6A). The limited but significant antitumor activity observed in FcγR-deficient animals was consistent with the inventors and others observations that the viral vector (FIG. 4A) and CTLA-4:B7 blockade per se—in the absence of Treg depletion (FIGS. 9F and 12A)—delayed tumor growth but contributed only limited survival advantage.

Besides affording immune effector-mediated ADCC and ADCP of antibody-coated target cells, FcγRs have been shown to promote tumor antigen cross-presentation (DiLillo and Ravetch, 2015), broadening and enhancing the CD8⁺ T cell antitumor response to encompass normally excluded MHCII-restricted extracellular tumor antigens. The inventor's findings that i.t. VV_{GM}-αCTLA4 antitumor immunity was FcγR-dependent, and induced more robust expansion of tumor-specific CD8⁺ T cells and additionally induced virus-specific CD8⁺ T cells, indicated that it might also promote tumor antigen cross-presentation. This notion was reinforced by differential gene expression analyses of tumors harvested from VV_{GM}-αCTLA4-injected compared to viral backbone (VV)-injected and untreated CT26 tumor-bearing mice (FIG. 6B and FIG. 6C). Besides upregulating Batf per se, differential gene analyses demonstrated upregulation of Type I IFN responses and CD8α markers associated with antigen cross-presenting cDC1 dendritic cells. Additional signatures supported the above characterized CD8⁺ T cell-dependent (and potentially NK-cell-mediated granzyme-dependent) tumor cytolysis underlying triggering and actuation of VV_{GM}-αCTLA4 antitumor immunity.

To assess a role for antigen cross-presentation in VV_{GM}-αCTLA4-induced antitumor immunity the inventors used mice lacking the transcription factor Batf3 (Batf3^{-/-} mice). Batf3^{-/-} mice lack CD8α⁺ dendritic cells and as a consequence show defective antigen cross-presentation and severely impaired CD8⁺ T cell responses to viruses during infection, and to tumor antigens in mouse experimental models of cancer (Hildner et al., 2008). Further, cDC1s and antigen cross-presentation are known to mediate therapeutic activity of immune checkpoint blockers including αCTLA-4 (Gubin et al., 2014). The inventors therefore compared antitumor activity of i.t. VV_{GM}-αCTLA4 in Batf3⁺/+ and Batf3^{-/-} C57BL/6 mice transplanted with MC38 tumors. Strikingly, Batf3-deficiency abrogated i.t. VV_{GM}-αCTLA4 antitumor immunity as demonstrated by 0/8 Batf3^{-/-} compared to 9/9 WT mice surviving (FIG. 6D).

Collectively, these results demonstrated that VV_{GM}-αCTLA4 has both FcγR-dependent and cDC1-dependent antitumor activity, identifying intratumorally induced Treg-depletion and tumor antigen cross-presentation as major mechanisms, and intratumoral CTLA-4:B7-blockade and oncolysis as supporting mechanisms, underlying i.t. VV_{GM}-αCTLA4 induced CD8⁺ T cell antitumor immunity.

Intratumoral Anti-CTLA-4-VV Expands Peripheral Effector CD8⁺ T Cells and Reduces Treg and Exhausted CD8⁺ T Cells

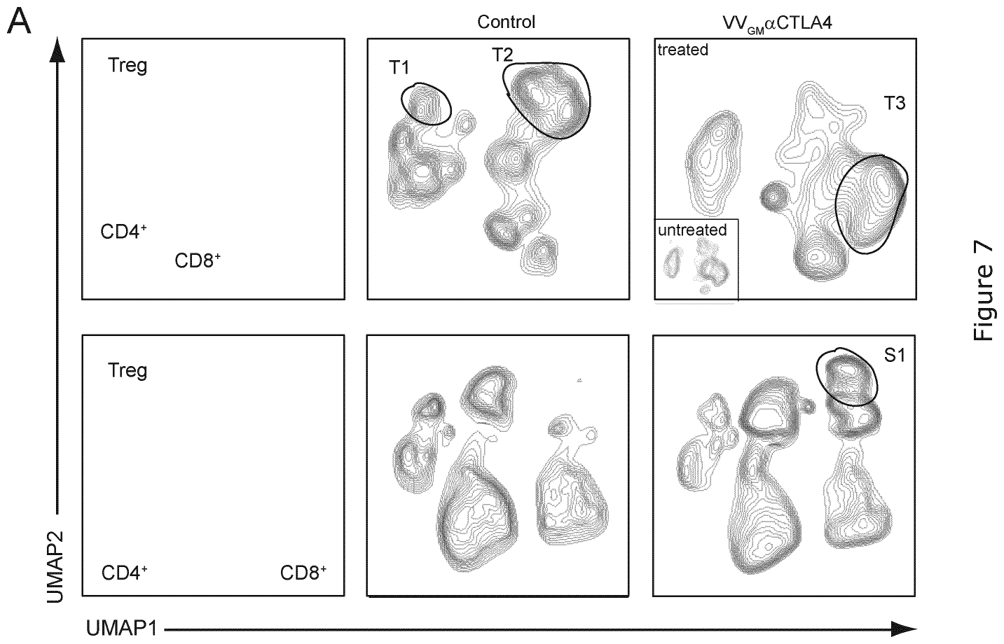

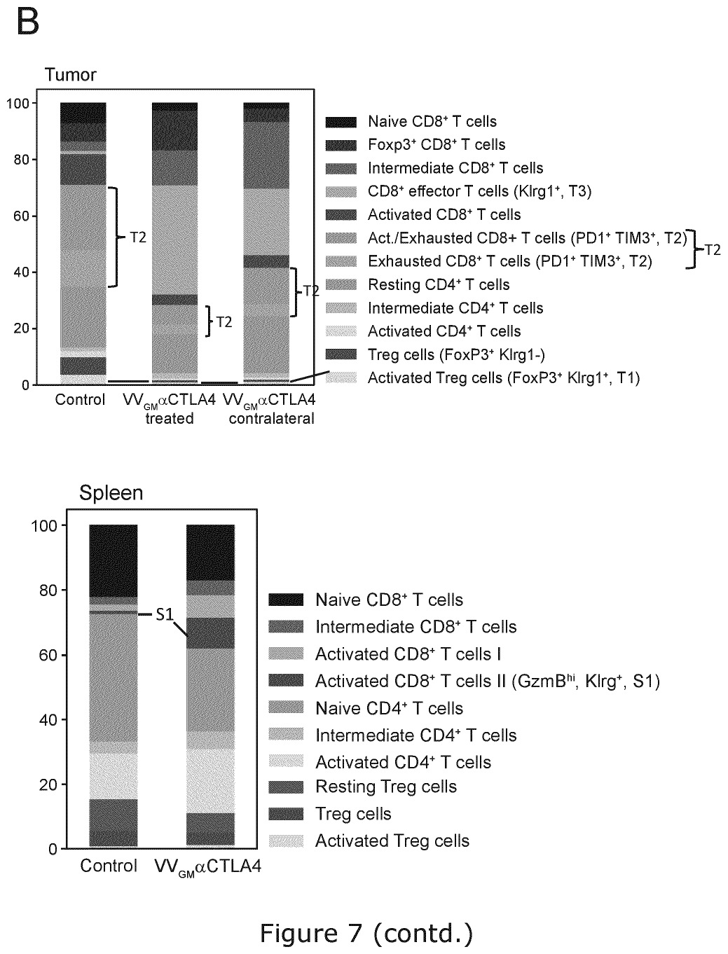

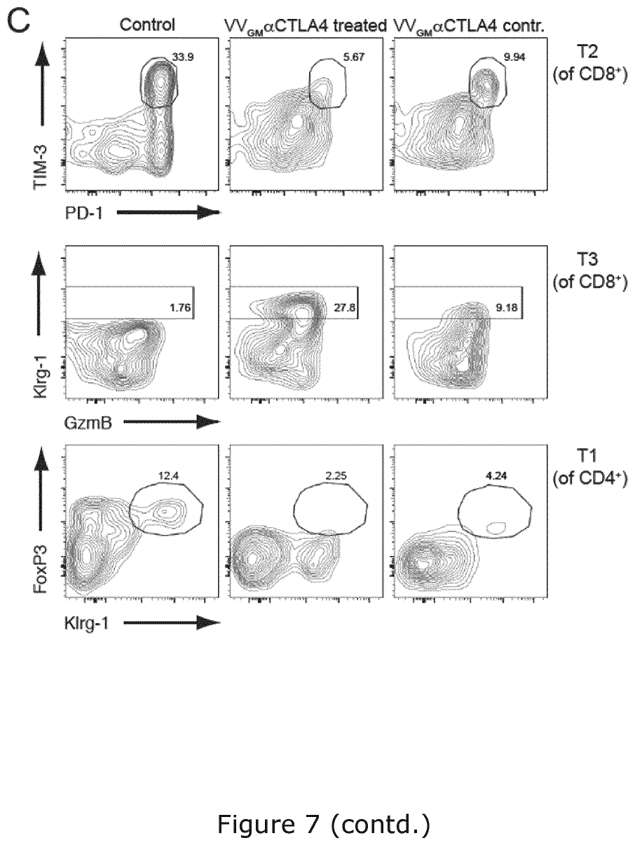

The inventors proceeded to qualitatively characterize how i.t. VV_{GM}-αCTLA4 modulates TIL responses in injected and flanking tumors, and in the periphery. Using multicolour flow-cytometry, and a high-dimensional antibody panel designed to identify functionally distinct anti-tumor and pro-tumor TIL subsets, 12 T cell clusters across treatment groups were identified (FIG. 7 and FIG. 13C). Strikingly, i.t. VV_{GM}-αCTLA4 eliminated exhausted (PD-1⁺ TIM-3⁺) CD8⁺ T cells and robustly expanded non-exhausted Klrg1⁺ effector CD8⁺ T cells in injected tumors compared to mock-treated animals (FIG. 7). At the same time, and consistent with the findings above, i.t. VV_{GM}-αCTLA4 effectively depleted CTLA-4⁺ intratumoral Tregs, including Klrg1⁺ Tregs, which are known to express high levels of CTLA-4 and to be particularly suppressive (Nakagawa et al., 2016) (FIG. 7).

Assessment of flanking distal tumors, which had not been injected with antibody-encoding virus, revealed similar but less profound modulation of TIL by i.t. VV_{GM}-αCTLA4. Further, and in keeping with the inventor's observations that intratumoral administration of the αCTLA4-encoding oncolytic virus expanded tumor-specific CD8⁺ T cells in the periphery (FIG. 5B-5D), i.t. VV_{GM}-αCTLA4 induced activated Granzyme B⁺ (Klrg1⁺) CD8⁺ T cell subsets in spleen (FIG. 7A). Finally, and consistent with the antibody-encoding virus achieving tumor-restricted Treg depletion, Treg populations that were depleted in tumor beds were largely unaltered in spleen by i.t. VV_{GM}-αCTLA4 (FIG. 7A).

Intratumoral Anti-CTLA-4-VV Combines with Anti-PD-1 to Reject “Cold” Distal Tumors

The inventor's observations demonstrated that VV_{GM}-αCTLA4 acted locally in injected tumors, principally by mechanisms involving anti-CTLA-4 mAb-dependent tumor antigen cross-presentation and Treg-depletion, to “ignite” systemic adaptive antitumor immunity and robust peripheral tumor-specific CD8⁺ T cell expansion. These findings indicated that VV_{GM}-αCTLA4 might synergize with therapeutic agents that help mobilize CD8⁺ T cells to the tumor. Anti-PD-1 is thought to act principally by reversal of T cell exhaustion (Hui et al., 2017; Wei et al., 2018) and possibly by mobilizing stem-like memory CD8⁺ T cells to tumors (Galletti et al., 2020; Simon et al., 2020). Despite anti-PD-1's documented ability to improve survival in multiple solid cancers of different origin, anti-PD-1 does not improve outcome in patients with poorly immune infiltrated “cold tumors” (Galon and Bruni, 2019), which perhaps represent the greatest unmet medical need in cancer therapy today.

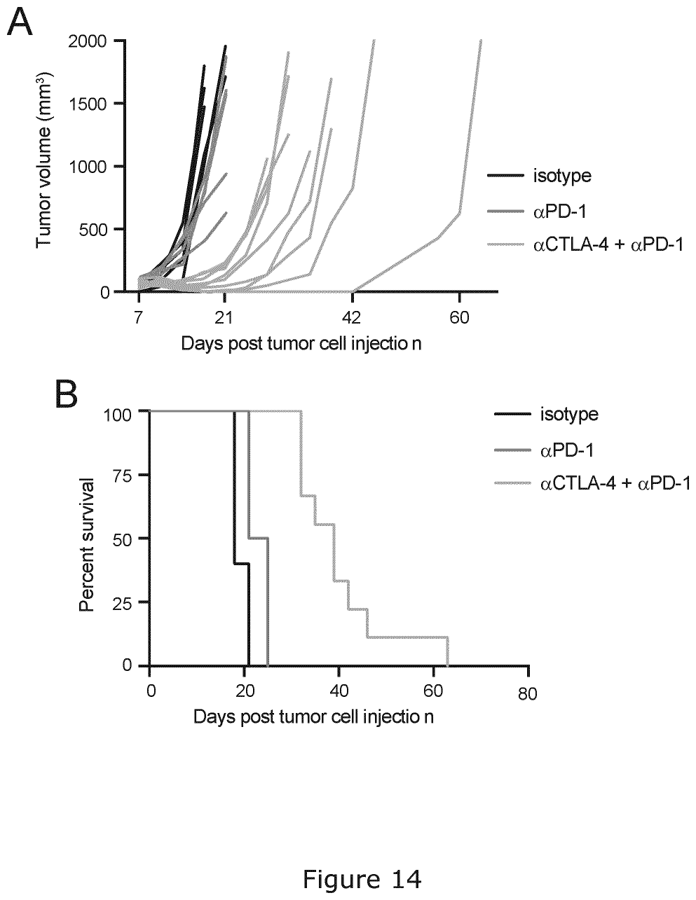

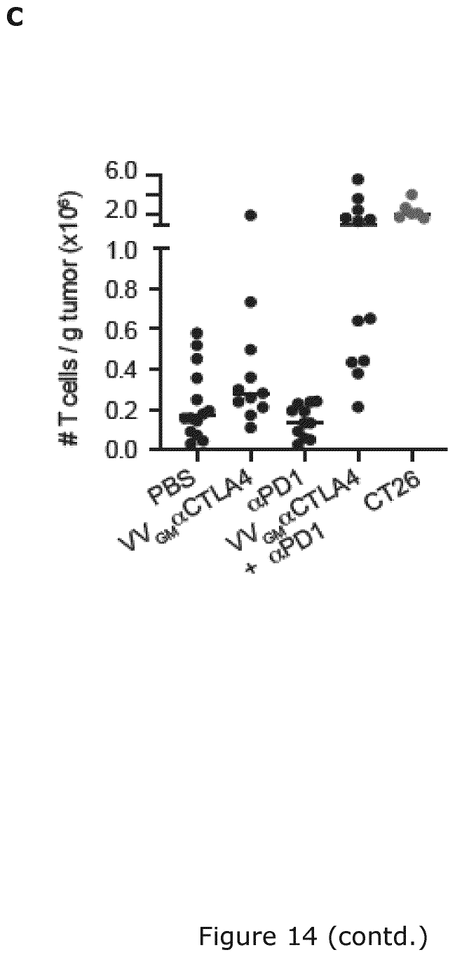

Based on their apparently different, and potentially complementary mechanism-of-action, the inventors therefore next examined synergizing effects of anti-PD-1 and VV_{GM}-αCTLA4 with a focus on ICB-resistant, poorly immune infiltrated and poorly immunogenic “cold” cancers using B16 C57BL/6 as a model system. Previous data had demonstrated that B16 tumors were refractory to ICB therapy, including clinically relevant systemic administration of anti-PD-1 (10 mg/kg), anti-CTLA-4 (10 mg/kg) or the combination thereof (FIG. 14).

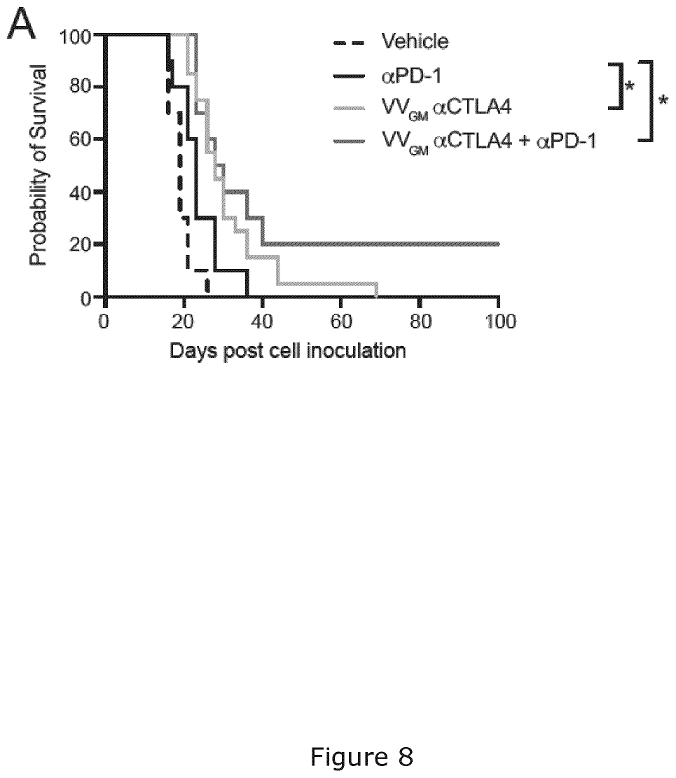

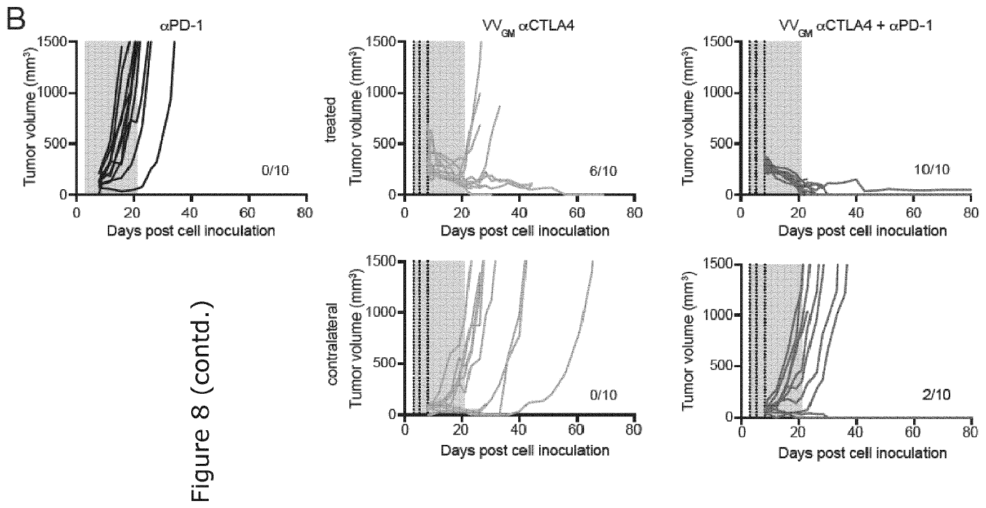

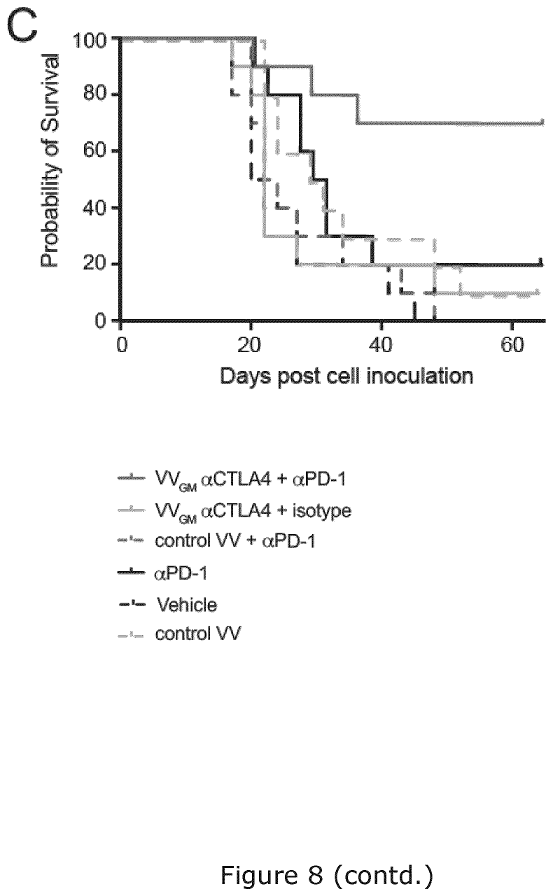

So as to mimic the clinical situation where palpable large tumors will be injected with the antibody-encoding virus, but small or undetectable metastasized lesions cannot be injected, the inventors established a twin-tumor B16/C57BL6 model where the animals carry one “large” and one “small” tumor, and where only the large tumor was injected i.t. with VV_{GM}-αCTLA4. Resistance to anti-PD-1 was confirmed by lack of tumor growth inhibition or survival benefit following systemic treatment with a maximally efficacious dose of 10 mg/kg (FIG. 8A).

As previously observed, single agent treatment with VV_{GM}-αCTLA4 significantly reduced tumor growth of the primary injected tumor (FIGS. 4 and 8B). However, i.t. VV_{GM}-αCTLA4 only induced a slight delay of uninjected tumor's growth (FIG. 8B), which did not translate into animal survival (FIG. 8A). Strikingly then, and in contrast to single agent or combined anti-PD-1 and anti-CTLA-4 therapy, combined treatment with i.t. VV_{GM}-αCTLA4 and systemic anti-PD-1 significantly inhibited injected and uninjected tumor growth, resulting in ˜20% of animals bearing cured in this ICB treatment-resistant model of “cold” cancer (FIG. 8A).

Further consistent with VV_{GM}-αCTLA4 being able to convert cold, ICB resistant, tumors toward an inflamed, ICB responsive, phenotype, combined treatment with VV_{GM}-αCTLA4 (but not anti-PD-1 alone) induced a strong influx of T cells into B16 tumors, which became similarly densely T cell rich compared to inflamed CT26 tumors (FIG. 14C).

These indicated synergizing effects of combined i.t. VV_{GM}-αCTLA4 and systemic anti-PD-1 were confirmed in BALB/c mice transplanted with syngeneic A20 tumors. This model was shown to be semi-responsive to anti-PD-1 with ˜20% of animals being cured by full therapeutic i.p. dosing (10 mg/kg) (FIG. 8C). While optimal therapeutic dosing with i.t. VV_{GM}-αCTLA4 was fully protective (10/10 animals cured, FIG. 4), sub-optimal treatment with 1/100 of the optimal dose showed insignificant tumor growth inhibition and no survival advantage. When therapeutic i.p. anti-PD-1 and subtherapeutic i.t. VV_{GM}-αCTLA4 were combined, the majority of animals (7/10) were cured (FIG. 8C).

### DISCUSSION

The inventors provide in vivo proof-of-concept that intratumoral administration of oncovirally encoded Treg-depleting αCTLA-4 has stronger and broader antitumor activity compared with approved systemic αCTLA-4 regimens, yet through its tumor-restricted nature of exposure is indicated to be safe and well-tolerated. I.t. VV_{GM}-αCTLA4 induced stronger expansion of tumor-specific CD8⁺ T cells compared with systemic recombinant αCTLA-4, and had antitumor activity in poorly immune infiltrated “cold” syngeneic mouse tumor models resistant to clinically relevant dosing with systemic αCTLA-4 and αPD-1. Remarkably, our observations suggest that the potent systemic antitumor immunity induced by oncoviral αCTLA-4 derived strictly from “immune igniting” effects in injected tumors; i.t. VV_{GM}-αCTLA4 was not associated with virus spread or antibody exposure to distal un-injected tumors, but rather achieved tumor-restricted CTLA-4 receptor saturation and Treg depletion.

These observations have important implications for both the expected clinical efficacy and tolerability of i.t. VV_{GM}-αCTLA4. From an efficacy perspective, they demonstrate that local administration of oncovirally-encoded αCTLA-4 may provide greater therapeutic benefit compared with available (ipilimumab), and Treg-depletion-optimized (Arce Vargas, Furness et al. 2018) or “masked” (Gutierrez, Long et al. 2020), systemic αCTLA-4 antibody regimens, as well as compared with previously described oncolytic virus approaches encoding non-Treg depletion-optimized αCTLA-4 (Aroldi, Sacco et al. 2020). At a mechanistic level, VV_{GM}-αCTLA4 induced FcγR-dependent Treg depletion and cDC1⁺ antigen cross-presentation are likely to underly both the observed robust CD8⁺ T cell expansion and synergism with αPD-1 to reject cold tumors. Besides mediating induction of endogenous anti-tumor immune responses (Hildner, et al. 2008) and efficacy of systemic checkpoint blockade therapy (Gubin, Zhang et al. 2014, Salmon, Idoyaga et al. 2016, Garris, Arlauckas et al. 2018), cDC1s promote the proliferative response of intratumoral CD8⁺ TILs, expand the pool of TCF1⁺ stem-like precursors, and induce generation of TIM3⁺ terminal effectors during αPD-1 therapy (Mao, et al. 2021).

Similarly, Treg depletion achieved with mAb to co-stimulatory or co-inhibitory receptors e.g. IL-2R and CTLA-4 may promote CD8⁺ effector function and synergize with αPD-1 (Wei, et al. 2019, Solomon, et al. 2020). With regards development of “dual activity” immune modulatory antibodies that reduce Treg and expand antitumor CD8⁺ T cells, accumulating data demonstrate the importance of both target biology, fine-tuning of effector CD8⁺ T cell-enhancing and Treg-depleting properties, as well as delivery regimen. For example, it was recently demonstrated that FcγR-competent non-ligand blocking antibodies to IL-2R, which deplete Treg but do not starve CD8⁺ effector T cells of critical (IL-2-mediated) growth survival signaling, have superior therapeutic potential in cancer therapy compared with ligand-blocking αIL-2R antibodies (Solomon, et al. 2020).

Analogously, but differently, the inventors recently reported that antibodies to 4-1BB can be made to deplete Tregs or promote effector T cell expansion by antibody isotype switching (altering FcγR-engagement), but that harnessing both mechanisms required sequential administration or hinge-engineering (Buchan et al., 2018). As described here in the context of oncolytic viral infection, spatial restriction of Treg depletion-enhanced function-blocking anti-CTLA-4 to injected tumors appears to be a particularly promising approach to harnessing maximal therapeutic activity of immune modulatory anti-CTLA-4 antibodies, when used alone or in combination with synergizing checkpoint blockade therapy e.g. anti-PD-1/L1.

Several observations support optimizing Treg-depletion in anti-CTLA-4 for tumor-localized therapy. Firstly, independent studies have established that therapeutic efficacy of anti-CTLA-4 depends on and correlates with Treg-depletion (Arce Vargas et al., 2018; Simpson et al., 2013). The herein presented data on therapeutic activity of Treg-depleting anti-CTLA-4 clones, which showed strong curative effect in FcγR-proficient (Treg depleting) antibody Fc-formats and hosts compared with their FcγR-deficient (non-depleting) counterparts, support this notion. Second, while clinical outcome of melanoma patients treated with ipilimumab was recently reported to correlate with FcγR-engagement and Treg depletion, data from the inventors T cell humanized mouse model suggests ipilimumab has limited depleting activity compared to the herein vectorized anti-CTLA-4 antibody 4-E03 against human Treg cells expressing intratumorally relevant levels of CTLA-4. Furthermore, whereas clinically tolerated doses of ipilimumab (1 mg/kg to 3 mg/kg depending on indication and regimen) are associated only with sub-saturating CTLA-4 receptor occupancy and submaximal effect (Ribas et al., 2005) (Bertrand et al., 2015), the inventors data demonstrates that oncolytic vectorisation and i.t. administration can generate therapeutically optimal exposure (sustained CTLA-4 receptor saturation) in an apparently safe manner-even with Treg-depletion enhanced anti-CTLA-4. Finally, and supporting our vectorization of a Treg-depletion enhanced and checkpoint blocking “dual activity” αCTLA-4 antibody, antibody-mediated CTLA-4 blockade was recently shown to synergize with FcγR-dependent depletion in improving tumor-specific CD8⁺ T cell responses. Antibody blockade of CTLA-4 functionally destabilized intratumoral Treg and promoted B7:CD28 co-stimulation and antitumor CD8⁺ T effector function through processes involving altered glycolysis and competition for B7 ligands (Zappasodi, et al. 2021).

The fact that full therapeutic dosing achieved tumor-restricted anti-CTLA-4 exposure indicates that severe toxicities associated with sustained systemic Treg-depletion e.g. those observed in FoxP3-DTR mouse model (Kim et al., 2007) are unlikely to manifest. Similarly, since anti-CTLA-4 checkpoint blockade effects will be restricted to TILs with tumor-antigen specificity, untoward self-reactivity associated with systemic anti-CTLA-4 should be minimal. In contrast, but consistent with the well-documented central immune checkpoint nature of CTLA-4 (Chambers et al., 1996; Leach et al., 1996), anti-CTLA-4 side effects associated with systemic administration i.e. body-wide antibody exposure, may be of severe autoimmune nature and can have fatal consequences (Tivol et al., 1995; Waterhouse et al., 1995).

Taken together therefore, while efficacy and tolerability of anti-CTLA-4 has until now been considered to be linked and dose-dependent precluding use of full therapeutic doses of anti-CTLA-4 based regimens, the findings strongly suggest that spatial restriction of vectorized Treg depleting αCTLA-4 is able to overcome these current limitations, uncoupling efficacy from tolerability.

### REFERENCES

1. Hodi F S, O'Day S J, McDermott D F, Weber R W, Sosman J A, Haanen J B, et al. Improved survival with ipilimumab in patients with metastatic melanoma. N Engl J Med. 2010; 363 (8): 711-23.
2. Larkin J, Chiarion-Sileni V, Gonzalez R, Grob J J, Cowey C L, Lao C D, et al. Combined Nivolumab and Ipilimumab or Monotherapy in Untreated Melanoma. N Engl J Med. 2015; 373 (1): 23-34.
3. Topalian S L, Hodi F S, Brahmer J R, Gettinger S N, Smith D C, McDermott D F, et al. Safety, activity, and immune correlates of anti-PD-1 antibody in cancer. N Engl J Med. 2012; 366 (26): 2443-54.
4. Sharma P, Hu-Lieskovan S, Wargo J A, and Ribas A. Primary, Adaptive, and Acquired Resistance to Cancer Immunotherapy. Cell. 2017; 168 (4): 707-23.
5. Chen D S, and Mellman I. Oncology meets immunology: the cancer-immunity cycle. Immunity. 2013; 39 (1): 1-10.
6. Gajewski T F, Schreiber H, and Fu Y X. Innate and adaptive immune cells in the tumor microenvironment. Nat Immunol. 2013; 14 (10): 1014-22.
7. Wagner J, Wickman E, DeRenzo C, and Gottschalk S. CAR T Cell Therapy for Solid Tumors: Bright Future or Dark Reality? Mol Ther. 2020; 28 (11): 2320-39.
8. Ribas A, Dummer R, Puzanov I, VanderWalde A, Andtbacka R H I, Michielin O, et al. Oncolytic Virotherapy Promotes Intratumoral T Cell Infiltration and Improves Anti-PD-1 Immunotherapy. Cell. 2017; 170 (6): 1109-19 e10.
9. Wei S C, Sharma R, Anang N A S, Levine J H, Zhao Y, Mancuso J J, et al. Negative Co-stimulation Constrains T Cell Differentiation by Imposing Boundaries on Possible Cell States. Immunity. 2019; 50 (4): 1084-98.e10.
10. Arce Vargas F, Furness A J S, Litchfield K, Joshi K, Rosenthal R, Ghorani E, et al. Fc Effector Function Contributes to the Activity of Human Anti-CTLA-4 Antibodies. Cancer Cell. 2018; 33 (4): 649-63 e4.
11. Postow M A, Chesney J, Pavlick A C, Robert C, Grossmann K, McDermott D, et al. Nivolumab and ipilimumab versus ipilimumab in untreated melanoma. N Engl J Med. 2015; 372 (21): 2006-17.
12. Bertrand A, Kostine M, Barnetche T, Truchetet M E, and Schaeverbeke T. Immune related adverse events associated with anti-CTLA-4 antibodies: systematic review and meta-analysis. BMC Med. 2015; 13:211.
13. Tivol E A, Borriello F, Schweitzer A N, Lynch W P, Bluestone J A, and Sharpe A H. Loss of CTLA-4 leads to massive lymphoproliferation and fatal multiorgan tissue destruction, revealing a critical negative regulatory role of CTLA-4. Immunity. 1995; 3 (5): 541-7.
14. Marabelle A, Kohrt H, and Levy R. Intratumoral anti-CTLA-4 therapy: enhancing efficacy while avoiding toxicity. Clin Cancer Res. 2013; 19 (19): 5261-3.
15. Marabelle A, Kohrt H, Sagiv-Barfi I, Ajami B, Axtell R C, Zhou G, et al. Depleting tumor-specific Tregs at a single site eradicates disseminated tumors. J Clin Invest. 2013; 123 (6): 2447-63.
16. Waterhouse P, Penninger J M, Timms E, Wakeham A, Shahinian A, Lee K P, et al. Lymphoproliferative disorders with early lethality in mice deficient in Ctla-4. Science. 1995; 270 (5238): 985-8.
17. Leach D R, Krummel M F, and Allison J P. Enhancement of antitumor immunity by CTLA-4 blockade. Science. 1996; 271 (5256): 1734-6.
18. Peggs K S, Quezada S A, Chambers C A, Korman A J, and Allison J P. Blockade of CTLA-4 on both effector and regulatory T cell compartments contributes to the antitumor activity of anti-CTLA-4 antibodies. J Exp Med. 2009; 206 (8): 1717-25.
19. Simpson T R, Li F, Montalvo-Ortiz W, Sepulveda M A, Bergerhoff K, Arce F, et al. Fc-dependent depletion of tumor-infiltrating regulatory T cells co-defines the efficacy of anti-CTLA-4 therapy against melanoma. J Exp Med. 2013; 210 (9): 1695-710.
20. Ingram J R, Blomberg O S, Rashidian M, Ali L, Garforth S, Fedorov E, et al. Anti-CTLA-4 therapy requires an Fc domain for efficacy. Proc Natl Acad Sci USA. 2018; 115 (15): 3912-7.
21 Veitonmaki N, Hansson M, Zhan F, Sundberg A, Lofstedt T, Ljungars A, et al. A human ICAM-1 antibody isolated by a function-first approach has potent macrophage-dependent antimyeloma activity in vivo. Cancer Cell. 2013; 23 (4): 502-15.
22. Buchan S L, Dou L, Remer M, Booth S G, Dunn S N, Lai C, et al. Antibodies to Costimulatory Receptor 4-1B B Enhance Anti-tumor Immunity via T Regulatory Cell Depletion and Promotion of CD8 T Cell Effector Function. Immunity. 2018.
23. Fransen M F, van der Sluis T C, Ossendorp F, Arens R, and Melief C J. Controlled local delivery of CTLA-4 blocking antibody induces CD8+ T-cell-dependent tumor eradication and decreases risk of toxic side effects. Clin Cancer Res. 2013; 19 (19): 5381-9.
24 Bommareddy P K, Shettigar M, and Kaufman H L. Integrating oncolytic viruses in combination cancer immunotherapy. Nat Rev Immunol. 2018; 18 (8): 498-513.
25. Foloppe J, Kempf J, Futin N, Kintz J, Cordier P, Pichon C, et al. The Enhanced Tumor Specificity of TG6002, an Armed Oncolytic Vaccinia Virus Deleted in Two Genes Involved in Nucleotide Metabolism. Mol Ther Oncolytics. 2019; 14:1-14.
26. Kleinpeter P, Fend L, Thioudellet C, Geist M, Sfrontato N, Koerper V, et al. Vectorization in an oncolytic vaccinia virus of an antibody, a Fab and a scFv against programmed cell death-1 (PD-1) allows their intratumoral delivery and an improved tumor-growth inhibition. Oncoimmunology. 2016; 5 (10): e1220467.
27. Fend L, Yamazaki T, Remy C, Fahrner C, Gantzer M, Nourtier V, et al. Immune Checkpoint Blockade, Immunogenic Chemotherapy or IFN-alpha Blockade Boost the Local and Abscopal Effects of Oncolytic Virotherapy. Cancer Res. 2017; 77 (15): 4146-57.
28. Liu Z, Ravindranathan R, Kalinski P, Guo Z S, and Bartlett D L. Rational combination of oncolytic vaccinia virus and P D-L1 blockade works synergistically to enhance therapeutic efficacy. Nat Commun. 2017; 8:14754.
29. Marchand J-B, Semmrich M, Fend L, Tornberg U-C, Silvestre N, Frendéus B, et al. Society of Immunotherapy 33rd Annual meeting. National Harbour, MD: Journal of Cancer Therapy; 2018.
30. Grosso J F, and Jure-Kunkel M N. CTLA-4 blockade in tumor models: an overview of preclinical and translational research. Cancer Immun. 2013; 13:5.
31 Andtbacka R H, Kaufman H L, Collichio F, Amatruda T, Senzer N, Chesney J, et al. Talimogene Laherparepvec Improves Durable Response Rate in Patients With Advanced Melanoma. J Clin Oncol. 2015; 33 (25): 2780-8.
32 Chesney J, Puzanov I, Collichio F, Singh P, Milhem M M, Glaspy J, et al. Randomized, Open-Label Phase II Study Evaluating the Efficacy and Safety of Talimogene Laherparepvec in Combination With Ipilimumab Versus Ipilimumab Alone in Patients With Advanced, Unresectable Melanoma. J Clin Oncol. 2018; 36 (17): 1658-67.
33. Ngwa W, Irabor O C, Schoenfeld J D, Hesser J, Demaria S, and Formenti S C. Using immunotherapy to boost the abscopal effect. Nat Rev Cancer. 2018; 18 (5): 313-22.
34. DiLillo D J, and Ravetch J V. Differential Fc-Receptor Engagement Drives an Anti-tumor Vaccinal Effect. Cell. 2015; 161 (5): 1035-45.
35 Hildner K, Edelson B T, Purtha W E, Diamond M, Matsushita H, Kohyama M, et al. Batf3 deficiency reveals a critical role for CD8alpha+ dendritic cells in cytotoxic T cell immunity. Science. 2008; 322 (5904): 1097-100.
36 Gubin M M, Zhang X, Schuster H, Caron E, Ward J P, Noguchi T, et al. Checkpoint blockade cancer immunotherapy targets tumour-specific mutant antigens. Nature. 2014; 515 (7528): 577-81.
37 Nakagawa H, Sido J M, Reyes E E, Kiers V, Cantor H, and Kim H J. Instability of Helios-deficient Tregs is associated with conversion to a T-effector phenotype and enhanced antitumor immunity. Proc Natl Acad Sci USA. 2016; 113 (22): 6248-53.
38. Hui E, Cheung J, Zhu J, Su X, Taylor M J, Wallweber H A, et al. T cell costimulatory receptor CD28 is a primary target for PD-1-mediated inhibition. Science. 2017; 355 (6332): 1428-33.
39 Wei S C, Duffy C R, and Allison J P. Fundamental Mechanisms of Immune Checkpoint Blockade Therapy. Cancer Discov. 2018; 8 (9): 1069-86.
40. Simon S, Voillet V, Vignard V, Wu Z, Dabrowski C, Jouand N, et al. PD-1 and TIGIT coexpression identifies a circulating CD8 T cell subset predictive of response to anti-PD-1 therapy. J Immunother Cancer. 2020; 8 (2).
41. Galletti G, De Simone G, Mazza E M C, Puccio S, Mezzanotte C, Bi T M, et al. Two subsets of stem-like CD8 (+) memory T cell progenitors with distinct fate commitments in humans. Nat Immunol. 2020; 21 (12): 1552-62.
42. Galon J, and Bruni D. Approaches to treat immune hot, altered and cold tumours with combination immunotherapies. Nat Rev Drug Discov. 2019; 18 (3): 197-218.
43. Gutierrez M, Long G V, Friedman C F, Richards D A, Corr B, Bastos B R, et al. Anti-CTLA-4 probody BMS-986249 alone or in combination with nivolumab in patients with advanced cancers: Initial phase I results. Journal of Clinical Oncology. 2020; 38 (15_suppl): 3058-.
44. Aroldi F, Sacco J, Harrington K, Olsson-Brown A, Nanclares P, Menezes L, et al. 421 Initial results of a phase 1 trial of RP2, a first in class, enhanced potency, anti-CTLA-4 antibody expressing, oncolytic HSV as single agent and combined with nivolumab in patients with solid tumors. Journal for Immuno Therapy of Cancer. 2020; 8 (Suppl 3): A256-A7.
45. Garris C S, Arlauckas S P, Kohler R H, Trefny M P, Garren S, Piot C, et al. Successful Anti-PD-1 Cancer Immunotherapy Requires T Cell-Dendritic Cell Crosstalk Involving the Cytokines IFN-gamma and IL-12. Immunity. 2018; 49 (6): 1148-61 e7.
46 Salmon H, Idoyaga J, Rahman A, Leboeuf M, Remark R, Jordan S, et al. Expansion and Activation of CD103(+) Dendritic Cell Progenitors at the Tumor Site Enhances Tumor Responses to Therapeutic P D-L1 and BRAF Inhibition. Immunity. 2016; 44 (4): 924-38.
47. Mao T, Song E, and Iwasaki A. PD-1 blockade-driven anti-tumor CD8+ T cell immunity requires XCR1+ dendritic cells. bioRXIV. 2021.
48. Solomon I, Amann M, Goubier A, Vargas F A, Zervas D, Qing C, et al. CD25-Treg-depleting antibodies preserving IL-2 signaling on effector T cells enhance effector activation and antitumor immunity. Nat Cancer. 2020; 1 (12): 1153-66.
49. Wei S C, Anang NAS, Sharma R, Andrews M C, Reuben A, Levine J H, et al. Combination anti-CTLA-4 plus anti-PD-1 checkpoint blockade utilizes cellular mechanisms partially distinct from monotherapies. Proc Natl Acad Sci USA. 2019; 116 (45): 22699-709.
50 Ribas A, Camacho L H, Lopez-Berestein G, Pavlov D, Bulanhagui C A, Millham R, et al. Antitumor activity in melanoma and anti-self responses in a phase I trial with the anti-cytotoxic T lymphocyte-associated antigen 4 monoclonal antibody CP-675,206. J Clin Oncol. 2005; 23 (35): 8968-77.
51. Zappasodi R, Serganova I, Cohen I J, Maeda M, Shindo M, Senbabaoglu Y, et al. CTLA-4 blockade drives loss of Treg stability in glycolysis-low tumours. Nature. 2021; 591 (7851): 652-8.
52 Kim J M, Rasmussen J P, and Rudensky A Y. Regulatory T cells prevent catastrophic autoimmunity throughout the lifespan of mice. Nat Immunol. 2007; 8 (2): 191-7.
53. Chambers C A, Krummel M F, Boitel B, Hurwitz A, Sullivan T J, Fournier S, et al. The role of CTLA-4 in the regulation and initiation of T-cell responses. Immunol Rev. 1996; 153:27-46.
54. Binyamin L, Alpaugh R K, Hughes T L, Lutz C T, Campbell K S, and Weiner L M. Blocking N K cell inhibitory self-recognition promotes antibody-dependent cellular cytotoxicity in a model of anti-lymphoma therapy. J Immunol. 2008; 180 (9): 6392-401.
55. Chen T, Hoffmann K, Ostman S, Sandberg A S, and Olsson O. Identification of gliadin-binding peptides by phage display. BMC Biotechnol. 2011; 11:16.
56. Huang A Y, Gulden P H, Woods A S, Thomas M C, Tong C D, Wang W, et al. The immunodominant major histocompatibility complex class I-restricted antigen of a murine colon tumor derives from an endogenous retroviral gene product. Proc Natl Acad Sci USA. 1996; 93 (18): 9730-5.
57. Russell T A, and Tscharke D C. Strikingly poor CD8+ T-cell immunogenicity of vaccinia virus strain MVA in BALB/c mice. Immunol Cell Biol. 2014; 92 (5): 466-9.
58. Dobin A, Davis C A, Schlesinger F, Drenkow J, Zaleski C, Jha S, et al. STAR: ultrafast universal RNA-seq aligner. Bioinformatics. 2013; 29 (1): 15-21.
59 Smith T, Heger A, and Sudbery I. UMI-tools: modeling sequencing errors in Unique Molecular Identifiers to improve quantification accuracy. Genome Res. 2017; 27 (3): 491-9.
60. Anders S, Pyl P T, and Huber W. HTSeq—a Python framework to work with high-throughput sequencing data. Bioinformatics. 2015; 31 (2): 166-9.
61. Love M I, Huber W, and Anders S. Moderated estimation of fold change and dispersion for RNA-seq data with DESeq2. Genome Biol. 2014; 15 (12): 550.
62 Yu G, Wang L G, Han Y, and He Q Y. clusterProfiler: an R package for comparing biological themes among gene clusters. OMICS. 2012; 16 (5): 284-7.
63. Chan, W. M. and G. McFadden (2014). “Oncolytic Poxviruses.” Annu Rev Virol 1 (1): 119-141.
64. Marino, N. et al. (2017). “Development of a versatile oncolytic virus platform for local intra-tumoural expression of therapeutic transgenes.” PLOS One 12 (5): e0177810.
65. McDermott, D. F. et al. (2018). “Clinical activity and molecular correlates of response to atezolizumab alone or in combination with bevacizumab versus sunitinib in renal cell carcinoma.” Nat Med 24 (6): 749-757.
66. Mosely, S. I. et al. (2017). “Rational Selection of Syngeneic Preclinical Tumor Models for Immunotherapeutic Drug Discovery.” Cancer Immunol Res 5 (1): 29-41.
67. Tumeh, P. C. et al. (2014). “PD-1 blockade induces responses by inhibiting adaptive immune resistance.” Nature 515 (7528): 568-571.
68. Bruni, D. et al. (2020). “The immune contexture and Immunoscore in cancer prognosis and therapeutic efficacy.” Nature Rev Cancer 20:662-680.
69 Galon, J. et al. (2006). “Type, density and location of immune cells within human colorectal tumours predict clinical outcome.” Science. 313:1960-1964.
70 Fridman, W. H. et al. (2012). “The immune contexture in human tumours: impact on clinical outcome.” Nature Rev Cancer 12:298-306.
71. Fridman, W. H. et al. (2017). “The immune contexture in cancer prognosis and treatment.” Nature Rev Clin. Oncol, 14:717-334.
72. Hu, Z. et al. (2018). “Tumor-infiltrating CD45RO+ memory cells correlate with favorable prognosis in patients with lung adenocarcinoma. J. Thorac, Dis, 10:2089-2099.
73. Lanzi A. et al. (2020). “The consensus immunoscore: toward a new classification of colorectal cancer” OncoImmunology, 9:1-3.
74. Angell, H. K. et al. (2020). “The Immunoscore: Colon Cancer and Beyond.” Clinical Cancer Research 26:332-339.

1. (canceled)

2. (canceled)

3. A method for treating cancer in a patient, wherein the cancer comprises or consists of a cold tumor, the method comprising administering to the patient:
an oncolytic virus capable of expressing a first antibody molecule that specifically binds to CTLA-4; and
a second antibody molecule that specifically binds to PD-1 and/or PD-L1.

4. (canceled)

5. The method according to claim 3, wherein the cold tumor is treated by the first and second antibody molecule.

6. The method according to claim 3, wherein the antibody molecule that specifically binds to CTLA-4 is an Fcγ receptor engaging antibody or a Treg depleting antibody.

7. (canceled)

8. The method according to claim 3, wherein the oncolytic virus is an oncolytic poxvirus.

9. The method according to claim 8, wherein the poxvirus belongs to the Chordopoxvirinae subfamily, more preferably to the Orthopoxvirus genus preferably selected from the group consisting of vaccinia virus, cowpox virus, canarypox virus, ectromelia virus and myxoma virus.

10. The method according to claim 9, wherein the oncolytic virus is a vaccinia virus defective for both thymidine kinase (TK) and/or ribonucleotide reductase (RR) activities and comprising nucleotide sequences encoding SEQ ID NO: 20 and SEQ ID NO: 21 or SEQ ID NO: 53 and ID. NO: 54.

11. The method according to claim 9, wherein the vaccinia virus further comprises a nucleotide sequence encoding a GM-CSF, with a specific preference for a human GM-CSF (e.g. having SEQ ID NO: 55 or SEQ ID NO: 56) or a murine GM-CSF (e.g. having SEQ ID NO: 57 or SEQ ID NO: 58).

12. The method according to claim 3, wherein the virus comprises a nucleotide sequence encoding the heavy chain of the first antibody molecule inserted at the viral J2R locus and/or comprises a nucleotide sequence encoding the light chain of the first antibody molecule inserted at the viral I4L locus.

13. The method according to claim 3, wherein the first antibody molecule is selected from the group consisting of antibody molecules comprising 1-6 of the CDRs selected from the group consisting of SEQ ID NOs: 3, 6, 8, 10, 12 and 14.

14. The method according to claim 3, wherein the first antibody molecule is selected from the group consisting of antibody molecules comprising 1-6 of the CDRs VH-CDR1, VH-CDR2, VH-CDR3, VL-CDR1, and VL-CDR3,
wherein VH-CDR1, if present, is selected from the group consisting of SEQ ID NOs: 15, 22, 29 and 35;
wherein VH-CDR2, if present, is selected from the group consisting of SEQ ID NOs: 16, 23, 30, and 36;
wherein VH-CDR3, if present, is selected from the group consisting of SEQ ID NOs: 17, 24, 31 and 37;
wherein VL-CDR1, if present, is selected from the group consisting of SEQ ID NOs: 10 and 38;
wherein VL-CDR2, if present, is selected from the group consisting of SEQ ID NOs: 18, 25, 32 and 39;
wherein VL-CDR3, if present, is selected from the group consisting of SEQ ID NOs: 19, 26 and 40.

15. The method according to claim 3, wherein the first antibody molecule comprises the six CDRs having SEQ ID. NOs: 15, 16, 17, 10, 18 and 19, or the six CDRs having SEQ ID NOs: 22, 23, 24, 10, 25 and 26.

16. The method according to claim 3, wherein the first antibody molecule comprises a variable heavy chain selected from the group consisting of SEQ. ID. NOs: 20 and 27 and/or a variable light chain selected from the group consisting of SEQ ID NOs: 21 and 28.

17. The method according to claim 3, wherein the first antibody molecule comprises the heavy chain constant region SEQ ID NO: 43 and/or the light chain constant region SEQ ID NO: 44.

18. The method according to claim 3, wherein the first antibody molecule is selected from the group consisting of ipilimumab and tremelimumab.

19. The method according to claim 3, wherein the first antibody molecule is an antibody molecule that is capable of competing for binding to CTLA-4 with the first antibody molecule.

20. The method according to claim 3, wherein the first and/or second antibody molecule is selected from the group consisting of a full-size antibody, a chimeric antibody, a single chain antibody, a Fab, a Fv, an scFv, a Fab′, and a (Fab′)₂.

21. The method according to claim 3, wherein the first and/or second antibody molecule is selected from the group consisting of a human IgG antibody, a humanized IgG antibody and an IgG antibody of human origin.

22. The method according to claim 3, wherein the virus comprises a nucleotide sequence encoding the first antibody molecule.

23. The method according to claim 22, wherein the nucleotide sequence comprises or consists of a sequence selected from the group consisting of SEQ ID NOs: 45-52.

24. The method according to claim 3, wherein the second antibody molecule is selected from the group consisting of a human antibody molecule, a humanized antibody molecule, and an antibody molecule of human origin.

25. The method according to claim 3, wherein the second antibody molecule is a monoclonal antibody molecule or an antibody molecule of monoclonal origin.

26. The method according to claim 3, wherein the second antibody molecule is selected from the group consisting of a full-size antibody, a chimeric antibody, a single chain antibody, and an antigen-binding fragment thereof.

27. The method according to claim 3, wherein the second antibody molecule is a human IgG antibody, a humanized IgG antibody molecule or an IgG antibody molecule of human origin.

28. The method according to claim 3, wherein the second antibody molecule specifically binds to PD1, and is selected from the group consisting of pembrolizumab; nivolumab; cemiplimab; camrelizumab; spartalizumab; dostarlimab; tislelizumab; JTX-4014; sintilimab (IBI308); toripalimab (JS 001); AMP-224; and AMP-514 (MEDI0680), or wherein the second antibody molecule specifically binds to PD-L1, and is selected from the group consisting of atezolizumab; durvalumab; avelumab; CS1001; KN035 (envafolimab); and CK-301.

29. (canceled)

30. The method according to claim 3, wherein the cold tumor is an immune-deserted tumor; and/or an immune-excluded tumor; and/or a tumor with poor immune infiltration.

31. The method according to claim 3, wherein the cold tumor is selected from the group consisting of melanoma; pancreatic cancer; prostate cancer; colorectal cancer; hepatocellular carcinoma; lung cancer; bladder cancer; kidney cancer; gastric cancer; cervical cancer; Merkel cell carcinoma; ovarian cancer, head and neck cancer; mesothelioma; and breast cancer.

32. The method according to claim 3, wherein the cancer is: resistant to treatment with an antibody molecule that specifically binds to CTLA-4; and/or is resistant to treatment with an antibody molecule that specifically binds to PD1; and/or is resistant to treatment with an antibody molecule that specifically binds to PD-L1.

33. (canceled)

34. The method according to claim 3, wherein the patient has previously been treated with an antibody molecule that specifically binds to CTLA-4, and/or an antibody molecule that specifically binds to PD-1 and/or an antibody molecule that specifically binds to PD-L1, and wherein the patient has been found to be resistant to the treatment, or has become resistant to the treatment following or during the treatment.

35. (canceled)
